(19) United States
(12) Patent Application Publication
(10) Pub. No.: US 2025/0186613 A1
(43) Pub. Date: Jun. 12, 2025

(54) VIRAL VECTORS ENCODING GLP-2 RECEPTOR AGONIST FUSIONS AND USES THEREOF IN TREATING SHORT BOWEL SYNDROME

(71) Applicant: The Trustees of the University of Pennsylvania, Philadelphia, PA (US)

(72) Inventors: James M. Wilson, Philadelphia, PA (US); Christian Hinderer, Baltimore, MD (US); Makoto Horiuchi, Wallingford, PA (US)

(73) Assignee: The Trustees of the University of Pennsylvania, Philadelphia, PA (US)

(21) Appl. No.: 18/843,447

(22) PCT Filed: Mar. 3, 2023
(86) PCT No.: PCT/US2023/063681

§ 371 Date: Sep. 3, 2024

Related U.S. Application Data
(60) Provisional application No. 63/316,219, filed on Mar. 3, 2022.

(51) Int. Cl. A61K 48/00; A61K 9/00; A61K 38/17; A61K 38/26; A61P 1/00; C12N 15/86

(52) U.S. Cl. CPC A61K 48/005; A61K 9/0019; A61K 38/1774; A61K 38/26; A61K 48/0075; A61K 48/0083; A61P 1/00; C12N 15/86; C12N 2750/14143; C12N 2830/002

(57) ABSTRACT

Compositions and methods for treating short bowel syndrome in a subject are provided. A viral vector is provided which includes a nucleic acid molecule comprising a sequence encoding a GLP-2 receptor agonist fusion protein and regulatory sequences which direct expression thereof.

### REFERENCE TO AN ELECTRONIC SEQUENCE LISTING

The contents of the electronic sequence listing (22-10018PCT_Seq-Listing.xml; Size: 161 kb; and Date of Creation: Mar. 3, 2023) is herein incorporated by reference in its entirety.

### BACKGROUND OF THE INVENTION

Short bowel syndrome (SBS) is a rare organ failure caused by surgical resection of bowel due to congenital or acquired reasons. SBS is a major cause of intestinal failure (IF), the persistent reduction of gut function below the minimum necessary for the absorption of macro nutrients and/or water and electrolytes. Standard treatment of SBS/IF includes lifelong daily parenteral nutrition (PN), an intravenous infusion of special form of foods, to support daily required nutrient for patients. In addition to the burden and risk associated to conducting daily PN, PN causes complications including infection, gut hypoplasia, liver diseases, renal dysfunction, and bone diseases leading sever negative impact of the quality of life of SBS patients. Medications used for SBS had been those to treat only symptoms associated with SBS/IF until Teduglutide, a Glucagon-like peptide-2 (GLP-2) agonist, was developed and approved by FDA and EMA.

GLP-2 is a 33 amino acid intestine peptide hormone which has a potent intestinotrophic effect. GLP-2 indeed increases the length of small intestine and villi length of the intestinal epithelium leading to effective absorption of nutrient even with shorter remnant bowel of SBS patients and reduction of PN volume and numbers. However, native GLP-2 cannot be used as an effective injectable with its very short serum half-life due to the cleavage by DPP IV. DPP IV-resistant long acting GLP-2 agonists, including fusions with IgG Fc domain and serum albumin, have been developed as disease modifying agents for SBS. Teduglutide is a GLP-2 like peptide with the A2G mutation for DPP IV resistance, that extends serum half-life from 5 min to 1.5 hours. Daily 0.05 mg/kg subcutaneous Teduglutide injections results in sustained and continuous reduction of PN volume throughout 2 years of treatment. 90% of patients achieved >20% reduction of PN volume per week from the baseline, and 70% obtained >1 additional days PN off per week from the baseline with this treatment with a great safety profile (Schwartz et al., 2016). Daily Teduglutide injections are required for lifelong to maintain reduced PN volume and numbers (Compher et al., 2011).

What is needed are improved treatments for SBS.

### SUMMARY OF THE INVENTION

Viral vectors encoding glucagon-like peptide 2 (GLP-2) receptor agonist fusion protein constructs are provided herein. These viral vectors may achieve, in some embodiments, sustained expression of the GLP-2 receptor agonist in subjects and/or increased circulating half-life, as compared to vector-mediated delivery of a GLP-2 receptor agonist without a fusion partner. Further provided are methods of making and using such viral vectors.

In one aspect, a viral vector is provided which includes a nucleic acid comprising a polynucleotide sequence encoding a fusion protein. The fusion protein includes (a) a leader sequence comprising a secretion signal peptide, (b) a glucagon-like peptide-1 (GLP-2) receptor agonist, and (c) a fusion domain comprising (i) an IgG Fe or a functional variant thereof, (ii) an albumin or a functional variant thereof, or (iii) an XTEN polypeptide (Podust et al, 240:52-66 (October 2016). In one embodiment, the vector is an adeno-associated viral vector.

In one embodiment, the (i) the secretion signal peptide of the leader sequence comprises a thrombin signal peptide; (ii) the leader sequence comprises a thrombin propeptide; and/or (iii) the leader sequence comprises a thrombin leader sequence. In another embodiment, the leader sequence comprises an IL-2 leader sequence. In one embodiment, the GLP-fusion is selected from SEQ ID NO: 13, 15, 17, 19, 21, or 23, and functional variants thereof.

In one embodiment, the fusion domain is a human IgG4 Fe having the sequence of SEQ ID NO: 8, or a sequence sharing at least 90% identity therewith, or a functional variant thereof. In another embodiment, the fusion domain is a human albumin having the sequence of SEQ ID NO: 11, or a sequence sharing at least 90% identity therewith, or a functional variant thereof. In one embodiment, the fusion domain is a rhesus IgG4 Fe having the sequence of SEQ ID NO: 9, or a sequence sharing at least 90% identity therewith, or a functional variant thereof.

In another aspect, the viral vector includes an AAV capsid, and a vector genome packaged in the AAV capsid, said vector genome comprising AAV inverted terminal repeats (ITRs), the polynucleotide sequence encoding the fusion protein, and regulatory sequences which direct expression of the fusion protein.

In another aspect, a pharmaceutical composition suitable for use in treating short bowel syndrome in a subject is provided. The composition includes an aqueous liquid and the viral vector as described herein. In one embodiment, the subject is a human.

In yet another aspect, use of a viral vector as described herein is provided for the manufacture of a medicament for treating a subject having short bowel syndrome, optionally diabetes.

In another aspect, a method of treating a subject having short bowel syndrome is provided. The method includes administering to the subject an effective amount of a viral vector or composition as described herein, Other aspects and advantages of the invention will be readily apparent from the following detailed description of the invention.

### BRIEF DESCRIPTION OF THE DRAWINGS

FIG. 1 is a schematic of the processing of proglucagon in vivo. The sequences of GLP-1 (SEQ ID NO: 4) and GLP-2 (SEQ ID NO: 1) are shown.

FIG. 2 is a table showing sequences of GLP-2 and analogs.

FIG. 3A is a schematic of the GLP2.G2.Fc construct, which includes an A2G substitution in the GLP2 amino acid sequence, a thrombin leader, and an IgG4 Fc fusion.

FIG. 3B is a schematic of the GLP2.G2.SA construct, which includes an A2G substitution in the GLP2 amino acid sequence, a thrombin leader, and a serum albumim fusion.

FIG. 3C is an alignment of the human (SEQ ID NO: 1), cyno (SEQ ID NO: 5), and mouse (SEQ ID NO: 6) GLP2 sequences.

FIG. 4A shows an elution profile of hGLP2-SA protein using albupure column purification.

FIG. 4B shows a gel with stained hGLP2-SA protein from FIG. 4A.

FIG. 5A shows hGLP2-SA levels in RagKO mice dosed intramuscularly with AAVrh91.CI.hGLP2.G2.SA.rBG at 1e11 gc/mouse.

FIG. 5B are two graphs showing small intestine length and weight for vehicle and vector treated mice as described in FIG. 5A.

FIG. 5C shows intestine histology for vehicle and vector treated mice as described in FIG. 5A. Vector treated mice intestine show healthier villi as compared to vehicle treated mice.

FIG. 6A shows hGLP2-SA and hGLP2-Fc levels in mice treated with AAVrh91.CI.hGLP2.G2.SA.rBG or AAVrh91.CI.hGLP2.G2.Fc.rBG at a dosage of 1e11 gc/mouse. GLP-2 levels for the Fc construct were higher after about study day 7.

FIG. 6B shows a potency assay for hGLP2 and hGLP2.Fc. EC50 was determined to be 0.4 nM for hGLP2 while EC=13.6 nM for the fusion protein.

FIG. 7 shows the study design for an experiment as described in Example 4.

FIGS. 8A-8C show the results of the experiment as described in Example 4. 2 NHPs were administered AAVrh91.CB7.CI.hGLP-2-Fc.rBG via intramuscular injection (IM) at a dose of 1×10¹³ (1e13) GC/kg (E185NG) and a of dose 5×10¹⁰ (1e10) GC/kg (BM239H).

FIG. 8A shows plasma level of GLP-2-Fc fusion protein. FIG. 8B shows serum citrulline, a biomarker of gut surface area. FIG. 8C shows detection of anti-GLP-2-Fc antibody in NHP serum at 1:100 dilution.

FIGS. 9A-9F show the results of the experiment as described in Example 5. Rag1KO female mice were treated with an injection of the vector AAVrh91.CB7.CI.hGLP-2-Fc.rBG at a dosage of 1×10¹⁰ GC mouse, 3×10¹⁰ GC/mouse, or 1×10¹¹ GC/mouse via IM route of administration. The study design is shown in FIG. 9A. FIG. 9B shows serum GLP2 levels, FIG. 9C shows body weights over time. FIG. 9D shows body weights at day 56. FIG. 9E shows small intestine (SI) length, while FIG. 9F shows SI weight.

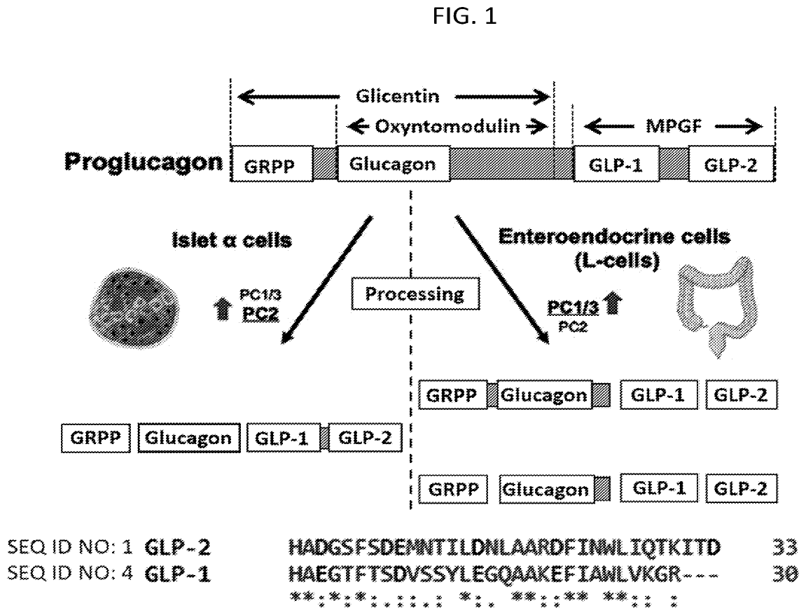

### DETAILED DESCRIPTION OF THE INVENTION

Described herein are adeno-associated viral (AAV) vectors expressing GLP-2 agonists to treat SBS/IF patients with a single intramuscular vector administration. Transgene GLP-2 agonists include the A2G mutation for DPP IV resistance and fusions with human IgG Fc domain or serum albumin for further extended serum half-life. In combination with these half-life extension technologies, the addition of a thrombin propeptide enables expression of GLP-2 agonists above the therapeutic level with remarkably lower vector doses (i.e., 1e10 to 1e12 GC/kg). Described herein are expression cassettes to express these proteins constitutively or in a controlled manner via administration of a small molecule drug that activates transcription of the GLP-2 agonist sequence.

Delivery of these constructs to subjects in need thereof via a number of routes, and particularly by expression in vivo mediated by a recombinant vector such as a rAAV vector, is described. Also provided are methods of using these constructs in regimens for treating short bowel syndrome in a subject in need thereof and increasing the half-life of GLP-2 in a subject. In addition, methods are provided for enhancing the activity of GLP-2 in a subject.

### GLP-2 Fusion Proteins

Post-translational processing of proglucagon generates glucagon-like peptide-2 (GLP-2), a 33-amino acid intestinotrophic peptide hormone, together with GLP-1. GLP-2 acts to slow gastric emptying, reduce gastric secretions and increase intestinal blood flow. GLP-2 also stimulates growth of the large and small intestine at least by enhancing crypt cell proliferation and villus length so as to increase the surface area of the mucosal epithelium. These effects suggest that GLP-2 can be used to treat a wide variety of gastrointestinal conditions. However, administering GLP-2 by itself to human patients has not shown promise. GLP-2 has a short half-life that limits its use as a therapeutic because rapid in vivo cleavage of GLP-2 by dipeptidyl peptidase IV (DPP-IV) yields an essentially inactive peptide. The amino acid sequence of human GLP-2 is

(SEQ ID NO: 1)
HADGSFSDEMNTILDNLAARDFINWLIQTKITD.

As discussed above, a GLP-2 analog, named teduglutide has been developed, in which amino acid residue 2 (alanine) has been substituted with glycine. The sequence of this GLP-2 analog is shown in SEQ ID NO: 2

HGDGSFSDEMNTILDNLAARDFINWLIQTKITD.

The disclosure provides fusion proteins comprising one or more copies of a GLP-2 receptor agonist, as well as polynucleotides and vectors encoding such fusion proteins. In some embodiments, the fusion protein comprises a polynucleotide sequence encoding a fusion protein comprising (a) a leader sequence comprising a secretion signal peptide, (b) a glucagon-like peptide-2 (GLP-2) receptor agonist, and (c) a fusion domain. In one embodiment, the GLP-2 receptor agonist comprises a thrombin leader sequence, a GLP-2 receptor agonist, and an IgG Fc or functional variant thereof. In another embodiment, the fusion protein comprises a thrombin leader, a GLP-2 receptor agonist, and an albumin or functional variant thereof. In another embodiment, the fusion protein comprises a thrombin leader, two copies of a GLP-2 receptor agonist, and an albumin or functional variant thereof.

In some embodiments, GLP-2 receptor agonists include variants which may include up to about 10% variation from a GLP-2 nucleic acid or amino acid sequence described herein or known in the art, which retain the function of the wild-type sequence. As used herein, by “retain function” it is meant that the nucleic acid or amino acid functions in the same way as the wild-type sequence, although not necessarily at the same level of expression or activity. For example, in one embodiment, a functional variant has increased expression or activity as compared to the wild-type sequence. In another embodiment, the functional variant has decreased expression or activity as compared to the wild-type sequence. In one embodiment, the functional variant has 10%, 20%, 30%, 40%, 50%, 60%, 70%, 80%, 90% or greater increase or decrease in expression or activity as compared to the wild-type sequence.

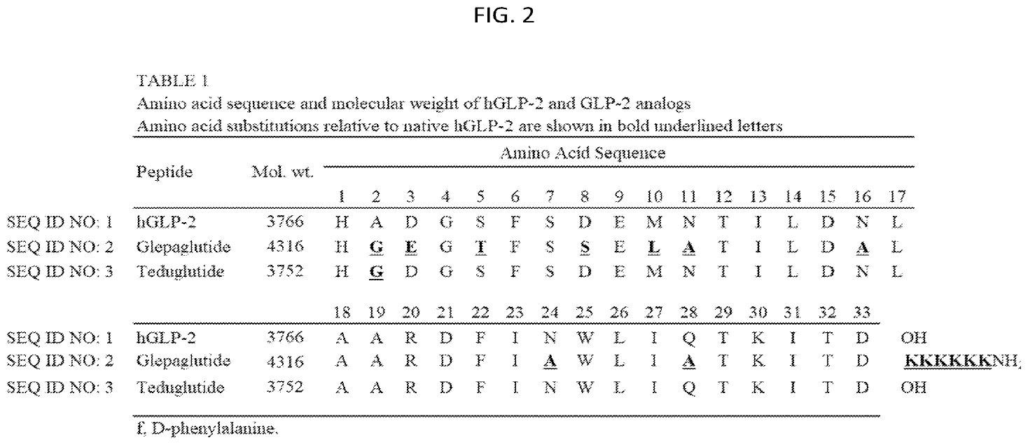

Other GLP-2 analogs have been developed and include glepaglutide (SEQ ID NO: 3), apraglutide, and others shown below, and in FIG. 2.

| Drug | Type | Company | Stage | T½ (h) | EC50 (/GLP2) | Comments |
|---|---|---|---|---|---|---|
| Teduglutide | Peptide | Shire | Approved | 1.5 (0.1) | 1 | |
| Glepaglutide | Peptide | Zealand | P3 | 50 | 4 | |
| Apraglutide | Peptide | Ferring | P3 | 30 | 0.5 | |
| HM-15912 | Fc fusion | Hanmi | P2 | ? | 5 | |
| GX-G8 | Fc fusion | Genexine | P1 | ? | ? | |
| TAK-681 | Fc fusion | Takeda (Shire) | P1 | 100 | 5 | |
| ZP7570 | Peptide | Zealand | P1 | ? | 10 | Dual acting |
| NB-1002 | XTEN fusion | Amunix | Pre | 240 | 100 | |

The fusion comprises, in one embodiment, a GLP-2 analog in combination with heterologous sequences. By GLP-2 analog is meant a polypeptide sharing at least 90%, 95%, 97%, 98%, 99% or 100% identity with native human GLP-2 (SEQ ID NO: 1) or GLP-2-A2G (SEQ ID NO: 2). In one embodiment, the GLP-2 analog has at most 1, 2, or 3 amino acid substitutions as compared to the native sequence. In another embodiment, the GLP-2 sequence is derived from a species other than human. For example, the GLP-2 may be from a non-human primate, dog, cat, mouse, rat, sheep, cow, horse, etc. In some embodiments, it is desirable to alter the native GLP-2 sequence to optimize one or more features thereof. In certain embodiments, the GLP-2 has a sequence which amino acid residue 2 (alanine) has been substituted with glycine, e.g., SEQ ID NO: 2. For example, in other embodiments, the GLP-2 analog contains one, two, three, 4, 5, 6, 7, 8 or up to 9 amino acid substitutions selected from A2G, D3E, S5T, D8S, M10L, N11A, N16A, N24A, Q28A as compared to the native sequence. These substitutions have been shown to improve efficacy of the clinical profile of GLP-2, including protection from DPP-4 inactivation (A2G). In one embodiment, the GLP-2 analog is a DPP-IV resistant variant of GLP-2. In one embodiment, the GLP-2 analog has a sequence comprising, or consisting of, SEQ ID NO: 2. In one embodiment, the variant shares at least 90% identity, 95% identity, 97% identity, 98% identity, 99% identity or 100% identity with SEQ ID NO: 2.

The fusion protein may comprise a leader sequence, which may comprise a secretion signal peptide. As used herein, the term “leader sequence” refers to any N-terminal sequence of a polypeptide.

The leader sequence may be derived from the same species for which administration is ultimately intended, e.g., a human. As used herein, the terms “derived” or “derived from” mean the sequence or protein is sourced from a specific subject species or shares the same sequence as a protein or sequence sourced from a specific subject species. For example, a leader sequence which is “derived from” a human, shares the same sequence (or a variant thereof, as defined herein) as the same leader sequence as expressed in a human. However, the specified nucleic acid or amino acid need not actually be sourced from a human. Various techniques are known in the art which are able to produce a desired sequence, including mutagenesis of a similar protein (e.g., a homolog) or artificial production of a nucleic acid or amino acid sequence. The “derived” nucleic acid or amino acid retains the function of the same nucleic acid or amino acid in the species from which it is “derived”, regardless of actual source of the derived sequence.

The term “amino acid substitution” and its synonyms are intended to encompass modification of an amino acid sequence by replacement of an amino acid with another, substituting, amino acid. The substitution may be a conservative substitution. It may also be a non-conservative substitution. The term conservative, in referring to two amino acids, is intended to mean that the amino acids share a common property recognized by one of skill in the art. For example, amino acids having hydrophobic nonacidic side chains, amino acids having hydrophobic acidic side chains, amino acids having hydrophilic nonacidic side chains, amino acids having hydrophilic acidic side chains, and amino acids having hydrophilic basic side chains. Common properties may also be amino acids having hydrophobic side chains, amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Methods for replacing an amino acid are well known to the skilled in the art and include, but are not limited to, mutations of the nucleotide sequence encoding the amino acid sequence. Reference to “one or more” herein is intended to encompass the individual embodiments of, for example, 1, 2, 3, 4, 5, 6, or more.

In one embodiment, the leader is a human thrombin (Factor II) sequence. In one embodiment, the thrombin leader has the sequence shown in SEQ ID NO: 7: MAHVRGLQLPGCLALAALCSLVHSQHVFLAPQQARSLLQRVRR, or a functional variant thereof having at most 1, 2, or 3 amino acid substitutions.

In one embodiment, functional variants of the desired leader include variants which may include up to about 10% variation from a leader nucleic acid or amino acid sequence described herein or known in the art, which retain the function of the wild type sequence.

In some embodiments, the coding regions for both the propeptide and GLP-2 peptide are incorporated into a single nucleic acid sequence without a linker between the coding sequences of the propeptide and GLP-2.

The fusion protein further includes a fusion domain. The fusion domain, in one embodiment, is a human IgG Fc fragment or a functional variant thereof. Immunoglobulins typically have long circulating half-lives in vivo. By fusing the GLP-2 receptor agonist (and leader) to an IgG Fc, the circulation time of the fusion protein is prolonged, while the function of the GLP-2 is preserved. In another embodiment, the fusion domain is a rhesus IgG Fc fragment or functional variant thereof.

As used herein, the Fc portion of an immunoglobulin has the meaning commonly given to the term in the field of immunology. Specifically, this term refers to an antibody fragment which does not contain the two antigen binding regions (the Fab fragments) from the antibody. The Fc portion consists of the constant region of an antibody from both heavy chains, which associate through non-covalent interactions and disulfide bonds. The Fc portion can include the hinge regions and extend through the CH2 and CH3 domains to the c-terminus of the antibody. The Fc portion can further include one or more glycosylation sites.

In one embodiment, the fusion domain is a human IgG Fc. The four subclasses, IgG1, IgG2, IgG3, and IgG4, which are highly conserved, differ in their constant region, particularly in their hinges and upper CH2 domains. See, Vidarsson et al, IgG Subclasses and Allotypes: From Structure to Effector Functions, Front Immunol. October 2014; 5: 520, which is incorporated herein by reference. The Fc domain can be derived from any human IgG, including human IgG1, human IgG2, human IgG3, or human IgG4. In one embodiment, the human IgG Fe is an IgG4 Fc. In one embodiment, the human IgG Fc is SEQ ID NO: 8: AESKYGPPCPPCPAPEAAGGPSVFLFPPKPKDTLMISRTPEVTCVVVDVSQEDP EVQFNWYVDGVEVHNAKTKPREEQFNSTYRVVSVLTVLHQDWLNGKEYKCKVSN KGLPSSIEKTISKAKGQPREPQVYTLPPSQEEMTKNQVSLTCLVKGFYPSDIAVEWES NGQPENNYKTTPPVLDSDGSFFLYSRLTVDKSRWQEGNVFSCSVMHEALHNHYTQK SLSLSLG. In another embodiment, the human IgG Fc shares at least 90% identity, at least 95% identity, at least 99% identity, or at least 100% identity to SEQ ID NO: 8.

In another embodiment, the fusion domain is a rhesus IgG Fc. The Fc domain can be derived from any rhesus IgG, including rhesus IgG1, rhesus IgG2, rhesus IgG3, or rhesus IgG4. In one embodiment, the rhesus IgG Fe is an IgG4 Fe. In one embodiment, the rhesus IgG Fc is SEQ ID NO: 9: PPCPPCPAPE LLGGPSVFLF PPKPKDTLMI SRTPEVTCVV VDVSQEDPEV QFNWYVDGVE VHNAQTKPRE RQFNSTYRVV SVLTVTHQDW LNGKEYTCKV SNKGLPAPIE KTISKAKGQP REPQVYILPP PQEELTKNQV SLTCLVTGFY PSDIAVEWES NGQPENTYKT TPPVLDSDGS YLLYSKLTVN KSRWQPGNIF TCSVMHEALH NHYTQKSLSV SPGK. In another embodiment, the rhesus IgG Fc shares at least 90% identity, at least 95% identity, at least 99% identity, or at least 100% identity to SEQ ID NO: 9. In one embodiment, the rhesus IgG further comprises a hinge sequence.

In another embodiment, the fusion domain is a human albumin or a functional variant thereof. In one embodiment, the human albumin is SEQ ID NO: 10: DAHKSEVAHRFKDLGEENFKALVLIAFAQYLQQCPFEDHVKLVNEVTEFAKTCVAD ESAENCDKSLHTLFGDKLCTVATLRETYGEMADCCAKQEPERNECFLQHKDDNPNL PRLVRPEVDVMCTAFHDNEETFLKKYLYEIARRHPYFYAPELLFFAKRYKAAFTECC QAADKAACLLPKLDELRDEGKASSAKQRLKCASLQKFGERAFKAWAVARLSQRFP KAEFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEYARR HPDYSVVLLLRLAKTYETTLEKCCAAADPHECYAKVFDEFKPLVEEPQNLIKQNCEL FEQLGEYKFQNALLVRYTKKVPQVSTPTLVEVSRNLGKVGSKCCKHPEAKRMPCAE DYLSVVLNQLCVLHEKTPVSDRVTKCCTESLVNRRPCFSALEVDETYVPKEFNAETF TFHADICTLSEKERQIKKQTALVELVKHKPKATKEQLKAVMDDFAAFVEKCCKADD KETCFAEEGKKLVAASQAALGL. In another embodiment, the human albumin shares at least 90% identity, at least 95% identity, at least 99% identity, or at least 100% identity to SEQ ID NO: 10.

In another embodiment, the fusion domain is a rhesus albumin or a functional variant thereof. In one embodiment, the rhesus albumin is SEQ ID NO: 11: DTHKSEVAHRFKDLGEEHFKGLVLVAFSQYLQQCPFEEHVKLVNEVTEFAKTCVAD ESAENCDKSLHTLFGDKLCTVATLRETYGEMADCCAKQEPERNECFLQHKDDNPNL PPLVRPEVDVMCTAFHDNEATFLKKYLYEVARRHPYFYAPELLFFAARYKAAFAEC CQAADKAACLLPKLDELRDEGKASSAKQRLKCASLQKFGDRAFKAWAVARLSQKF PKAEFAEVSKLVTDLTKVHTECCHGDLLECADDRADLAKYMCENQDSISSKLKECC DKPLLEKSHCLAEVENDEMPADLPSLAADYVESKDVCKNYAEAKDVFLGMFLYEY ARRHPDYSVMLLLRLAKAYEATLEKCCAAADPHECYAKVFDEFQPLVEEPQNLVKQ NCELFEQLGEYKFQNALLVRYTKKVPQVSTPTLVEVSRNLGKVGAKCCKLPEAKRM PCAEDYLSVVLNRLCVLHEKTPVSEKVTKCCTESLVNRRPCFSALELDEAYVPKAFN AETFTFHADMCTLSEKEKQVKKQTALVELVKHKPKATKEQLKGVMDNFAAFVEKC CKADDKEACFAEEGPKFVAASQAALA. In another embodiment, the rhesus albumin shares at least 90% identity, at least 95% identity, at least 99% identity, or at least 100% identity to SEQ ID NO: 11.

The in vivo function and stability of the fusion proteins of the present disclosure may be optimized by adding small peptide linkers, e.g., to prevent potentially unwanted domain interactions or for other reasons. Further, a glycine-rich linker may provide some structural flexibility such that the GLP-2 analog portion can interact productively with the GLP-2 receptor on target cells such as the beta cells of the pancreas. Thus, the C-terminus of the GLP-2 analog and the N-terminus of the fusion domain of the fusion protein are, in one embodiment, fused via a linker. In one embodiment, the linker includes 1, 1.5 or 2 repeats of a G-rich peptide linker having the sequence GGGGSGGGGSGGGGS (SEQ ID NO: 12).

In one embodiment, the fusion protein comprises (a) human thrombin leader, (b) a DPP-IV resistant variant of GLP-2(A2G), a linker, and (c) a human IgG Fc. In one embodiment, the fusion protein has the sequence of SEQ ID NO: 13, or a sequence at least 90%, at least 95%, at least 98%, or at least 99% identical thereto.

SEQ ID NO: 13
MAHVRGLQLPGCLALAALCSLVHSQHVFLAPQQARSLLQRVRRHG
DGSFSDEMNTILDNLAARDFINWLIQTKITDGGGGGGSGGGGSGG
GGSAESKYGPPCPPCPAPEAAGGPSVFLFPPKPKDTLMISRTPEV
TCVVVDVSQEDPEVQFNWYVDGVEVHNAKTKPREEQFNSTYRVVS
VLTVLHQDWLNGKEYKCKVSNKGLPSSIEKTISKAKGQPREPQVY
TLPPSQEEMTKNQVSLTCLVKGFYPSDIAVEWESNGQPENNYKTT
PPVLDSDGSFFLYSRLTVDKSRWQEGNVFSCSVMHEALHNHYTQK
SLSLSLG*

In one embodiment, the sequence encoding the fusion protein is SEQ ID NO: 14 or a sequence at least 75%, at least 85%, at least 90%, at least 95%, at least 98%, or at least 99% identical thereto.

SEQ ID NO: 14:
ATGGCTCACGTTCGGGGACTGCAGCTGCCTGGATGTCTGGCTCTT
GCCGCTCTGTGTAGCCTGGTGCACAGCCAGCACGTGTTTCTGGCT
CCTCAGCAAGCCAGATCACTGCTGCAGAGAGTTAGAAGGCACGGC
GACGGCAGCTTCAGCGACGAGATGAACACCATCCTGGACAACCTG
GCCGCCAGAGACTTCATCAACTGGCTGATCCAGACCAAGATCACC
GACGGTGGCGGAGGCGGAGGATCTGGTGGTGGTGGATCTGGCGGC
GGAGGTTCTGCCGAGTCTAAGTACGGACCTCCTTGTCCTCCCTGT
CCTGCTCCAGAAGCTGCTGGCGGCCCATCCGTGTTTCTGTTCCCT
CCAAAGCCTAAGGACACCCTGATGATCAGCAGAACCCCTGAAGTG
ACCTGCGTGGTGGTCGACGTGTCCCAAGAGGATCCTGAGGTGCAG
TTCAATTGGTACGTGGACGGCGTGGAAGTGCACAACGCCAAGACC
AAGCCTAGAGAGGAACAGTTCAACAGCACCTACAGAGTGGTGTCC
GTGCTGACCGTGCTGCACCAGGATTGGCTGAACGGCAAAGAGTAC
AAGTGCAAGGTGTCCAACAAGGGCCTGCCTAGCAGCATCGAGAAA
ACCATCAGCAAGGCCAAGGGCCAGCCAAGAGAACCCCAGGTGTAC
ACACTGCCTCCAAGCCAAGAGGAAATGACCAAGAACCAGGTGTCC
CTGACCTGCCTGGTCAAGGGCTTCTACCCTTCCGATATCGCCGTG
GAATGGGAGAGCAACGGCCAGCCTGAGAACAACTACAAGACCACA
CCTCCTGTGCTGGACTCCGATGGCTCATTCTTCCTGTACAGCAGA
CTGACCGTGGACAAGAGCAGGTGGCAAGAGGGCAACGTGTTCAGC
TGCAGCGTGATGCACGAGGCCCTGCACAACCACTACACCCAGAAA
AGCCTGAGCCTGTCTCTGGGCTAA

In one embodiment, the fusion protein comprises (a) human thrombin leader, (b) a DPP-IV resistant variant of GLP-2(A2G), a linker, and (c) a human serum albumin. In one embodiment, the fusion protein has the sequence of SEQ ID NO: 15, or a sequence at least 90%, at least 95%, at least 98%, or at least 99% identical thereto.

SEQ ID NO: 15:
MAHVRGLQLPGCLALAALCSLVHSQHVFLAPQQARSLLQRVRRHG
DGSFSDEMNTILDNLAARDFINWLIQTKITDGGGGGGSGGGGSGG
GGSDAHKSEVAHRFKDLGEENFKALVLIAFAQYLQQCPFEDHVKL
VNEVTEFAKTCVADESAENCDKSLHTLFGDKLCTVATLRETYGEM
ADCCAKQEPERNECFLQHKDDNPNLPRLVRPEVDVMCTAFHDNEE
TFLKKYLYEIARRHPYFYAPELLFFAKRYKAAFTECCQAADKAAC
LLPKLDELRDEGKASSAKQRLKCASLQKFGERAFKAWAVARLSQR
FPKAEFAEVSKLVTDLTKVHTECCHGDLLECADDRADLAKYICEN
QDSISSKLKECCEKPLLEKSHCIAEVENDEMPADLPSLAADFVES
KDVCKNYAEAKDVFLGMFLYEYARRHPDYSVVLLLRLAKTYETTL
EKCCAAADPHECYAKVFDEFKPLVEEPQNLIKQNCELFEQLGEYK
FQNALLVRYTKKVPQVSTPTLVEVSRNLGKVGSKCCKHPEAKRMP
CAEDYLSVVLNQLCVLHEKTPVSDRVTKCCTESLVNRRPCFSALE
VDETYVPKEFNAETFTFHADICTLSEKERQIKKQTALVELVKHKP
KATKEQLKAVMDDFAAFVEKCCKADDKETCFAEEGKKLVAASQAA
LG*

In one embodiment, the sequence encoding the fusion protein is SEQ ID NO: 16 or a sequence at least 75%, at least 85%, at least 90%, at least 95%, at least 98%, or at least 99% identical thereto.

SEQ ID NO: 16:
ATGGCTCACGTTCGGGGACTGCAGCTGCCTGGATGTCTGGCTCTT
GCCGCTCTGTGTAGCCTGGTGCACAGCCAGCACGTGTTTCTGGCT
CCTCAGCAAGCCAGATCACTGCTGCAGAGAGTTAGAAGGCACGGC
GACGGCAGCTTCAGCGACGAGATGAACACCATCCTGGACAACCTG
GCCGCCAGAGACTTCATCAACTGGCTGATCCAGACCAAGATCACC
GACGGTGGCGGAGGCGGAGGATCTGGTGGTGGTGGATCTGGCGGC
GGAGGTTCTGACGCCCACAAATCTGAAGTGGCCCACCGGTTCAAG
GACCTGGGCGAAGAGAATTTCAAGGCCCTGGTGCTGATCGCCTTC
GCTCAGTACCTGCAGCAGTGCCCCTTCGAGGACCACGTGAAGCTG
GTCAACGAAGTGACCGAGTTCGCCAAGACCTGCGTGGCCGACGAG
AGCGCCGAGAACTGTGATAAGAGCCTGCACACCCTGTTCGGCGAC
AAGCTGTGTACAGTGGCCACACTGAGAGAAACCTACGGCGAGATG
GCCGACTGCTGCGCCAAGCAAGAGCCCGAGAGAAACGAGTGCTTC
CTGCAGCACAAGGACGACAACCCCAACCTGCCTAGACTCGTGCGG
CCTGAAGTGGACGTGATGTGCACCGCCTTCCACGACAACGAGGAA
ACCTTCCTGAAGAAGTACCTGTACGAGATCGCCAGACGGCACCCC
TACTTTTACGCCCCTGAGCTGCTGTTCTTCGCCAAGCGGTATAAG
GCCGCCTTCACCGAGTGTTGTCAGGCCGCTGATAAGGCTGCCTGC
CTGCTGCCTAAGCTGGACGAGCTTAGAGACGAGGGCAAAGCCAGC
TCCGCCAAGCAGAGACTGAAGTGTGCCAGCCTGCAGAAGTTCGGC
GAGAGAGCCTTTAAGGCCTGGGCCGTTGCTAGACTGAGCCAGAGA
TTTCCCAAGGCCGAGTTTGCCGAGGTGTCCAAGCTCGTGACCGAC
CTGACAAAGGTGCACACCGAGTGCTGCCACGGCGACCTGCTGGAA
TGCGCCGACGATAGAGCCGACCTGGCCAAGTACATCTGCGAGAAC
CAGGACAGCATCAGCAGCAAGCTGAAAGAGTGCTGCGAGAAGCCT
CTGCTGGAAAAGAGCCACTGTATCGCCGAGGTGGAAAACGACGAG
ATGCCCGCCGATCTGCCTTCTCTGGCCGCCGATTTTGTGGAAAGC
AAGGACGTGTGCAAGAACTACGCCGAGGCCAAGGACGTGTTCCTG
GGCATGTTTCTGTACGAGTACGCCCGCAGACACCCCGACTACTCT
GTTGTGCTGCTGCTGAGACTGGCCAAAACCTACGAGACAACCCTG
GAAAAGTGCTGTGCCGCCGCTGATCCTCACGAGTGTTACGCCAAG
GTGTTCGACGAGTTCAAGCCACTGGTGGAAGAACCCCAGAACCTG
ATCAAGCAGAACTGCGAGCTGTTCGAGCAGCTGGGCGAGTACAAG
TTCCAGAACGCCCTGCTCGTGCGGTACACCAAGAAGGTGCCCCAG
GTTTCCACACCTACACTGGTTGAGGTGTCCCGGAACCTGGGCAAA
GTGGGCAGCAAGTGTTGCAAGCACCCTGAGGCCAAGAGAATGCCC
TGCGCCGAGGATTACCTGAGCGTGGTGCTGAATCAGCTGTGCGTG
CTGCACGAGAAAACCCCTGTGTCCGACAGAGTGACCAAGTGCTGT
ACCGAGAGCCTGGTCAACAGACGGCCTTGCTTTAGCGCCCTCGAG
GTGGACGAGACATACGTGCCCAAAGAGTTCAACGCCGAGACATTC
ACCTTCCACGCCGACATCTGTACCCTGAGCGAGAAAGAGCGGCAG
ATCAAGAAACAGACTGCCCTGGTGGAACTGGTCAAGCACAAGCCC
AAGGCCACCAAAGAACAGCTGAAGGCCGTGATGGACGACTTCGCC
GCCTTCGTGGAAAAGTGCTGCAAGGCCGACGACAAAGAGACCTGC
TTCGCCGAAGAGGGCAAGAAACTGGTGGCCGCTTCTCAGGCTGCT
CTGGGATAA

In one embodiment, the fusion protein comprises (a) human thrombin leader, (b) a DPP-IV resistant variant of GLP-2(A2G), a linker, and (c) an XTEN polypeptide. In one embodiment, the fusion protein has the sequence of SEQ ID NO: 17, or a sequence at least 90%, at least 95%, at least 98%, or at least 99% identical thereto.

SEQ ID NO: 17:
MAHVRGLQLPGCLALAALCSLVHSQHVFLAPQQARSLLQRVRRHG
DGSFSDEMNTILDNLAARDFINWLIQTKITDGGGGGGSGGGGSGG
GGSSPAGSPTSTEEGTSESATPESGPGTSTEPSEGSAPGSPAGSP
TSTEEGTSTEPSEGSAPGTSTEPSEGSAPGTSESATPESGPGSEP
ATSGSETPGSEPATSGSETPGSPAGSPTSTEEGTSESATPESGPG
TSTEPSEGSAPGTSTEPSEGSAPGSPAGSPTSTEEGTSTEPSEGS
APGTSTEPSEGSAPGTSESATPESGPGTSTEPSEGSAPGTSESAT
PESGPGSEPATSGSETPGTSTEPSEGSAPGTSTEPSEGSAPGTSE
SATPESGPGTSESATPESGPGSPAGSPTSTEEGTSESATPESGPG
SEPATSGSETPGTSESATPESGPGTSTEPSEGSAPGTSTEPSEGS
APGTSTEPSEGSAPGTSTEPSEGSAPGTSTEPSEGSAPGTSTEPS
EGSAPGSPAGSPTSTEEGTSTEPSEGSAPGTSESATPESGPGSEP
ATSGSETPGTSESATPESGPGSEPATSGSETPGTSESATPESGPG
TSTEPSEGSAPGTSESATPESGPGSPAGSPTSTEEGSPAGSPTST
EEGSPAGSPTSTEEGTSESATPESGPGTSTEPSEGSAPGTSESAT
PESGPGSEPATSGSETPGTSESATPESGPGSEPATSGSETPGTSE
SATPESGPGTSTEPSEGSAPGSPAGSPTSTEEGTSESATPESGPG
SEPATSGSETPGTSESATPESGPGSPAGSPTSTEEGSPAGSPTST
EEGTSTEPSEGSAPGTSESATPESGPGTSESATPESGPGTSESAT
PESGPGSEPATSGSETPGSEPATSGSETPGSPAGSPTSTEEGTST
EPSEGSAPGTSTEPSEGSAPGSEPATSGSETPGTSESATPESGPG
TSTEPSEGSAPG*

In one embodiment, the sequence encoding the fusion protein is SEQ ID NO: 18 or a sequence at least 75%, at least 85%, at least 90%, at least 95%, at least 98%, or at least 99% identical thereto.

SEQ ID NO: 18:
ATGGCTCACGTTCGGGGACTGCAGCTGCCTGGATGTCTGGCTCTT
GCCGCTCTGTGTAGCCTGGTGCACAGCCAGCACGTGTTTCTGGCT
CCTCAGCAAGCCAGATCACTGCTGCAGAGAGTTAGAAGGCACGGC
GACGGCAGCTTCAGCGACGAGATGAACACCATCCTGGACAACCTG
GCCGCCAGAGACTTCATCAACTGGCTGATCCAGACCAAGATCACC
GACGGTGGCGGAGGCGGAGGATCTGGTGGTGGTGGATCTGGCGGC
GGAGGAAGTTCTCCTGCTGGCAGCCCTACAAGCACCGAGGAAGGC
ACAAGCGAGTCTGCCACACCTGAGTCTGGCCCTGGCACATCTACA
GAGCCTAGCGAAGGATCTGCCCCAGGATCTCCTGCCGGCTCTCCA
ACATCTACCGAAGAGGGAACCAGCACCGAGCCATCTGAGGGATCT
GCTCCCGGAACAAGCACAGAGCCTTCAGAAGGATCCGCTCCTGGC
ACCTCTGAAAGCGCCACACCAGAAAGCGGACCTGGATCTGAGCCT
GCCACAAGCGGATCTGAGACACCTGGAAGCGAGCCAGCCACATCT
GGCAGCGAAACACCTGGTTCTCCAGCCGGATCTCCCACCAGCACA
GAAGAGGGCACATCCGAATCTGCTACCCCTGAATCTGGACCAGGC
ACCTCCACAGAACCTTCCGAGGGTTCTGCCCCTGGAACCTCTACC
GAACCATCAGAAGGCAGCGCTCCAGGTTCACCAGCTGGAAGCCCA
ACCTCTACAGAGGAAGGGACATCCACTGAGCCAAGCGAGGGAAGC
GCTCCCGGCACTAGTACAGAACCGAGCGAGGGCAGTGCTCCTGGA
ACCAGCGAATCCGCTACTCCAGAGAGTGGCCCAGGCACCAGTACT
GAACCCTCTGAGGGTAGCGCACCCGGAACATCTGAGAGCGCTACT
CCCGAATCAGGCCCAGGCTCTGAACCTGCTACCAGCGGAAGTGAA
ACACCCGGCACCTCTACTGAGCCCTCCGAAGGCTCAGCACCTGGC
ACAAGCACTGAACCATCAGAGGGCTCCGCACCAGGCACCAGCGAA
AGTGCTACACCAGAGTCAGGACCCGGAACCTCCGAAAGTGCAACT
CCTGAGAGCGGACCAGGCTCTCCCGCTGGATCTCCTACATCAACT
GAAGAAGGGACCTCCGAGAGCGCAACCCCAGAGTCTGGTCCAGGA
TCAGAACCTGCCACCTCCGGCTCTGAAACCCCAGGCACTTCTGAG
TCCGCCACGCCAGAATCTGGTCCTGGGACTAGCACCGAACCGAGT
GAAGGTTCAGCTCCCGGGACTTCTACGGAACCCAGTGAAGGATCT
GCACCCGGCACATCAACCGAACCGTCAGAGGGATCAGCCCCTGGG
ACTTCCACAGAGCCGTCTGAGGGCAGCGCCCCAGGGACGTCTACA
GAACCATCTGAAGGATCAGCACCAGGGACCTCTACCGAGCCAAGT
GAAGGCAGTGCACCGGGAAGTCCAGCAGGCTCCCCTACAAGTACT
GAAGAGGGTACTAGCACGGAACCCAGCGAGGGTTCCGCTCCAGGG
ACATCTGAATCCGCAACTCCGGAATCCGGACCTGGCAGTGAACCA
GCTACATCCGGATCCGAGACTCCGGGAACCTCAGAATCAGCTACA
CCCGAGAGTGGACCTGGCTCCGAACCAGCAACTAGCGGCTCAGAA
ACTCCTGGGACAAGCGAGAGTGCAACACCCGAATCTGGACCTGGA
ACAAGTACTGAGCCCAGCGAAGGCAGCGCCCCTGGAACTTCTGAA
TCTGCCACTCCTGAAAGTGGCCCTGGAAGCCCTGCAGGCTCACCC
ACATCCACAGAAGAAGGATCACCAGCAGGCAGCCCCACTTCAACG
GAAGAGGGATCCCCAGCTGGATCCCCAACTAGTACGGAAGAAGGC
ACTTCAGAAAGCGCTACGCCCGAGTCCGGTCCTGGCACTTCTACT
GAACCATCCGAGGGAAGTGCCCCTGGCACTTCCGAGAGTGCTACA
CCTGAAAGCGGTCCCGGCTCTGAACCAGCCACTTCTGGATCTGAA
ACGCCCGGGACATCCGAGTCAGCAACGCCCGAAAGCGGCCCAGGT
TCCGAGCCGGCTACTAGTGGTTCAGAGACTCCAGGGACTTCCGAG
TCTGCTACTCCTGAGTCCGGACCGGGAACATCAACCGAGCCTTCC
GAAGGATCTGCACCTGGAAGCCCTGCCGGATCTCCTACCAGTACT
GAGGAAGGCACCTCAGAGTCTGCCACTCCAGAGTCAGGTCCTGGA
AGCGAACCTGCAACAAGCGGCAGCGAAACTCCAGGCACTAGCGAG
TCAGCTACCCCAGAATCAGGACCTGGATCTCCAGCAGGGTCCCCA
ACATCTACTGAGGAAGGCTCTCCTGCTGGCTCCCCTACCTCTACC
GAAGAGGGGACCTCAACAGAGCCATCCGAGGGGAGCGCACCTGGT
ACATCAGAGTCCGCAACTCCCGAGTCTGGCCCCGGAACTAGCGAA
TCTGCAACCCCGGAAAGTGGACCCGGGACGAGTGAATCAGCCACA
CCTGAATCCGGTCCAGGATCCGAGCCTGCAACTTCTGGAAGCGAG
ACACCAGGATCTGAGCCAGCTACGTCTGGCTCTGAGACTCCTGGA
TCTCCTGCTGGTAGTCCCACCTCCACTGAAGAGGGAACTTCCACC
GAACCGAGCGAGGGATCAGCACCAGGCACTAGCACAGAACCGTCC
GAAGGATCTGCTCCAGGCTCTGAACCCGCAACCTCCGGATCAGAA
ACCCCTGGAACATCCGAAAGCGCTACACCGGAAAGTGGCCCCGGA
ACCTCTACAGAACCTAGCGAGGGAAGCGCACCAGGATAA

In one embodiment, the fusion protein comprises (a) rhesus thrombin leader, (b) a DPP-IV resistant variant of GLP-2(A2G), a linker, and (c) a rhesus IgG Fc. In one embodiment, the fusion protein has the sequence of SEQ ID NO: 19, or a sequence at least 90%, at least 95%, at least 98%, or at least 99% identical thereto.

SEQ ID NO: 19
MAHVRGLQLPGCLALAALCSLVHSQHVFLAPQQALSLLQRVRRHGD
GSFSDEMNTVLVDNLATRDFINWLIQTKITDGGGGGGGSGGGGSG
GGGSAEFTPPCPPCPAPELLGGPSVFLFPPKPKDTLMISRTPEVT
CVVVDVSQEDPEVQFNWYVDGVEVHNAQTKPRERQFNSTYRVVSV
LTVTHQDWLNGKEYTCKVSNKGLPAPIEKTISKAKGQPREPQVYI
LPPPQEELTKNQVSLTCLVTGFYPSDIAVEWESNGQPENTYKTTP
PVLDSDGSYLLYSKLTVNKSRWQPGNIFTCSVMHEALHNHYTQKS
LSVSPG*

In one embodiment, the sequence encoding the fusion protein is SEQ ID NO: 20 or a sequence at least 75%, at least 85%, at least 90%, at least 95%, at least 98%, or at least 99% identical thereto.

SEQ ID NO: 20:
ATGGCTCACGTTCGGGGACTGCAGCTGCCTGGATGTCTGGCTCTT
GCCGCTCTGTGTAGCCTGGTGCACAGCCAGCATGTGTTTCTGGCT
CCTCAGCAGGCCCTGAGCCTGCTGCAAAGAGTTAGAAGGCACGGC
GACGGCAGCTTCAGCGACGAGATGAATACCGTGCTGGTGGACAAC
CTGGCCACCAGAGACTTCATCAACTGGCTGATCCAGACCAAGATC
ACCGACGGTGGTGGCGGAGGCGGAGGATCTGGTGGCGGTGGTTCT
GGCGGTGGCGGATCTGCTGAGTTTACCCCTCCTTGTCCTCCCTGT
CCTGCTCCAGAACTGCTCGGCGGACCTTCCGTGTTCCTGTTTCCT
CCAAAGCCTAAGGACACCCTGATGATCAGCAGAACCCCTGAAGTG
ACCTGCGTGGTGGTGGACGTGTCCCAAGAGGATCCTGAGGTGCAG
TTCAATTGGTACGTGGACGGCGTGGAAGTGCACAACGCCCAGACA
AAGCCCAGAGAGCGGCAGTTCAACAGCACCTACAGAGTGGTGTCC
GTGCTGACCGTGACACACCAGGATTGGCTGAACGGCAAAGAGTAC
ACCTGTAAAGTCTCCAACAAGGGCCTGCCTGCTCCTATCGAGAAA
ACCATCAGCAAGGCCAAGGGCCAGCCTAGAGAACCCCAGGTGTAC
ATCCTGCCTCCACCTCAAGAGGAACTGACCAAGAACCAGGTGTCC
CTGACCTGTCTGGTCACCGGCTTCTACCCTTCCGATATCGCCGTG
GAGTGGGAGAGCAACGGACAGCCCGAGAACACCTACAAGACCACA
CCTCCAGTGCTGGACAGCGACGGCTCTTACCTGCTGTACTCCAAG
CTGACAGTGAACAAGAGCCGGTGGCAGCCCGGCAACATCTTCACC
TGTTCTGTGATGCACGAGGCCCTGCACAACCACTACACCCAGAAA
AGCCTGAGCGTGTCCCCTGGATAA

In one embodiment, the fusion protein comprises (a) rhesus thrombin leader, (b) a DPP-IV resistant variant of GLP-2(A2G), a linker, and (c) a rhesus serum albumin. In one embodiment, the fusion protein has the sequence of SEQ ID NO: 21, or a sequence at least 90%, at least 95%, at least 98%, or at least 99% identical thereto.

SEQ ID NO: 21
MAHVRGLQLPGCLALAALCSLVHSQHVFLAPQQALSLLQRVRRHG
DGSFSDEMNTVLVDNLATRDFINWLIQTKITGGGGGGGSGGGGSG
GGGSDTHKSEVAHRFKDLGEEHFKGLVLVAFSQYLQQCPFEEHVK
LVNEVTEFAKTCVADESAENCDKSLHTLFGDKLCTVATLRETYGE
MADCCAKQEPERNECFLQHKDDNPNLPPLVRPEVDVMCTAFHDNE
ATFLKKYLYEVARRHPYFYAPELLFFAARYKAAFAECCQAADKAA
CLLPKLDELRDEGKASSAKQRLKCASLQKFGDRAFKAWAVARLSQ
KFPKAEFAEVSKLVTDLTKVHTECCHGDLLECADDRADLAKYMCE
NQDSISSKLKECCDKPLLEKSHCLAEVENDEMPADLPSLAADYVE
SKDVCKNYAEAKDVFLGMFLYEYARRHPDYSVMLLLRLAKAYEAT
LEKCCAAADPHECYAKVFDEFQPLVEEPQNLVKQNCELFEQLGEY
KFQNALLVRYTKKVPQVSTPTLVEVSRNLGKVGAKCCKLPEAKRM
PCAEDYLSVVLNRLCVLHEKTPVSEKVTKCCTESLVNRRPCFSAL
ELDEAYVPKAFNAETFTFHADMCTLSEKEKQVKKQTALVELVKHK
PKATKEQLKGVMDNFAAFVEKCCKADDKEACFAEEGPKFVAASQA
ALA

In one embodiment, the sequence encoding the fusion protein is SEQ ID NO: or a sequence at least 75%, at least 85%, at least 90%, at least 95%, at least 98%, or at least 99% identical thereto.

SEQ ID NO: 22:
ATGGCTCACGTTCGGGGACTGCAGCTGCCTGGATGTCTGGCTCTT
GCCGCTCTGTGTAGCCTGGTGCACAGCCAGCATGTGTTTCTGGCT
CCTCAGCAAGCCCTGAGCCTGCTGCAAAGAGTTAGAAGGCACGGC
GACGGCAGCTTCAGCGACGAGATGAATACCGTGCTGGTGGACAAC
CTGGCCACCAGAGACTTCATCAACTGGCTGATCCAGACCAAGATC
ACCGGTGGTGGCGGAGGCGGAGGATCTGGTGGCGGTGGTTCTGGC
GGTGGCGGATCTGATACACACAAGTCTGAGGTGGCCCACCGGTTC
AAGGACCTGGGCGAAGAACACTTCAAAGGCCTGGTGCTGGTCGCC
TTCAGCCAGTACCTGCAGCAGTGCCCTTTCGAGGAACACGTGAAG
CTGGTCAACGAAGTGACCGAGTTCGCCAAGACCTGCGTGGCCGAC
GAGAGCGCCGAGAACTGTGATAAGAGCCTGCACACCCTGTTCGGC
GACAAGCTGTGTACAGTGGCCACACTGAGAGAAACCTACGGCGAG
ATGGCCGACTGCTGCGCCAAGCAAGAGCCCGAGAGAAACGAGTGC
TTCCTGCAGCACAAGGACGACAACCCCAACCTGCCTCCACTCGTC
AGACCCGAAGTGGACGTGATGTGCACCGCCTTCCACGACAATGAG
GCCACCTTCCTGAAGAAATACCTGTACGAGGTGGCCAGACGGCAC
CCCTACTTTTACGCCCCTGAACTGCTGTTCTTTGCCGCCAGGTAC
AAGGCCGCCTTCGCCGAATGTTGTCAGGCCGCTGATAAGGCCGCT
TGCCTGCTGCCTAAGCTGGACGAGCTTAGAGACGAGGGCAAAGCC
AGCTCCGCCAAGCAGAGACTGAAGTGTGCCAGCCTGCAGAAGTTC
GGCGATAGAGCCTTTAAGGCCTGGGCCGTCGCTAGACTGAGCCAG
AAGTTTCCCAAGGCCGAGTTTGCCGAGGTGTCCAAGCTCGTGACC
GACCTGACAAAGGTGCACACCGAGTGCTGTCACGGCGACCTGCTG
GAATGCGCCGACGATAGAGCCGACCTGGCCAAGTACATGTGCGAG
AACCAGGACAGCATCAGCAGCAAGCTGAAAGAGTGCTGCGACAAG
CCTCTGCTGGAAAAGAGCCACTGTCTGGCCGAGGTGGAAAACGAC
GAGATGCCCGCCGATCTGCCTTCTCTGGCCGCCGATTACGTGGAA
AGCAAGGACGTGTGCAAGAACTACGCCGAGGCCAAGGACGTGTTC
CTGGGCATGTTTCTGTACGAGTACGCCCGCAGACACCCCGACTAC
TCTGTTATGCTGCTGCTGAGACTGGCCAAGGCCTACGAGGCCACT
CTGGAAAAGTGTTGTGCCGCCGCTGATCCCCACGAGTGTTACGCC
AAAGTGTTCGACGAGTTCCAGCCACTGGTGGAAGAACCCCAGAAC
CTGGTCAAGCAGAACTGCGAGCTGTTCGAGCAGCTGGGCGAGTAC
AAGTTCCAGAACGCCCTGCTCGTGCGGTACACCAAGAAGGTGCCC
CAGGTTTCCACACCTACACTGGTTGAGGTGTCCCGGAACCTGGGA
AAAGTGGGCGCCAAGTGTTGCAAGCTGCCTGAGGCCAAGAGAATG
CCCTGCGCCGAGGATTACCTGAGCGTGGTGCTGAACAGACTGTGC
GTGCTGCACGAGAAAACCCCTGTGTCCGAGAAAGTGACCAAGTGC
TGTACCGAGAGCCTGGTCAATCGGAGGCCTTGCTTTAGCGCCCTG
GAACTGGACGAGGCCTACGTGCCCAAGGCCTTCAACGCCGAGACA
TTCACCTTCCACGCCGACATGTGTACCCTGAGCGAGAAAGAAAAG
CAAGTGAAGAAACAGACAGCCCTGGTCGAGCTGGTTAAGCACAAG
CCTAAGGCCACCAAAGAACAACTGAAGGGCGTGATGGACAACTTC
GCCGCCTTTGTGGAAAAATGCTGCAAGGCCGACGACAAAGAGGCC
TGCTTCGCAGAAGAGGGCCCTAAGTTTGTGGCCGCCTCTCAAGCT
GCTCTGGCTTAA

In one embodiment, the fusion protein comprises (a) rhesus thrombin leader, (b) a DPP-IV resistant variant of GLP-2(A2G), a linker, and (c) an XTEN polypeptide. In one embodiment, the fusion protein has the sequence of SEQ ID NO: 23, or a sequence at least 90%, at least 95%, at least 98%, or at least 99% identical thereto.

SEQ ID NO: 23
MAHVRGLQLPGCLALAALCSLVHSQHVFLAPQQALSLLQRVRRHG
DGSESDEMNTVLVDNLATRDFINWLIQTKITDGGGGGGSGGGGSG
GGGSSPAGSPTSTEEGTSESATPESGPGTSTEPSEGSAPGSPAGS
PTSTEEGTSTEPSEGSAPGTSTEPSEGSAPGTSESATPESGPGSE
PATSGSETPGSEPATSGSETPGSPAGSPTSTEEGTSESATPESGP
GTSTEPSEGSAPGTSTEPSEGSAPGSPAGSPTSTEEGTSTEPSEG
SAPGTSTEPSEGSAPGTSESATPESGPGTSTEPSEGSAPGTSESA
TPESGPGSEPATSGSETPGTSTEPSEGSAPGTSTEPSEGSAPGTS
ESATPESGPGTSESATPESGPGSPAGSPTSTEEGTSESATPESGP
GSEPATSGSETPGTSESATPESGPGTSTEPSEGSAPGTSTEPSEG
SAPGTSTEPSEGSAPGTSTEPSEGSAPGTSTEPSEGSAPGTSTEP
SEGSAPGSPAGSPTSTEEGTSTEPSEGSAPGTSESATPESGPGSE
PATSGSETPGTSESATPESGPGSEPATSGSETPGTSESATPESGP
GTSTEPSEGSAPGTSESATPESGPGSPAGSPTSTEEGSPAGSPTS
TEEGSPAGSPTSTEEGTSESATPESGPGTSTEPSEGSAPGTSESA
TPESGPGSEPATSGSETPGTSESATPESGPGSEPATSGSETPGTS
ESATPESGPGTSTEPSEGSAPGSPAGSPTSTEEGTSESATPESGP
GSEPATSGSETPGTSESATPESGPGSPAGSPTSTEEGSPAGSPTS
TEEGTSTEPSEGSAPGTSESATPESGPGTSESATPESGPGTSESA
TPESGPGSEPATSGSETPGSEPATSGSETPGSPAGSPTSTEEGTS
TEPSEGSAPGTSTEPSEGSAPGSEPATSGSETPGTSESATPESGP
GTSTEPSEGSAPG

In one embodiment, the sequence encoding the fusion protein is SEQ ID NO: 24 or a sequence at least 75%, at least 85%, at least 90%, at least 95%, at least 98%, or at least 99% identical thereto.

SEQ ID NO: 24:
ATGGCTCACGTTCGGGGACTGCAGCTGCCTGGATGTCTGGCTCTT
GCCGCTCTGTGTAGCCTGGTGCACAGCCAGCATGTGTTTCTGGCT
CCTCAGCAAGCCCTGAGCCTGCTGCAAAGAGTTAGAAGGCACGGC
GACGGCAGCTTCAGCGACGAGATGAATACCGTGCTGGTGGACAAC
CTGGCCACCAGAGACTTCATCAACTGGCTGATCCAGACCAAGATC
ACCGACGGTGGCGGAGGCGGAGGATCTGGTGGTGGTGGATCTGGC
GGCGGAGGAAGTTCTCCTGCTGGCAGCCCTACAAGCACCGAGGAA
GGCACAAGCGAGTCTGCCACACCTGAGTCTGGCCCTGGCACATCT
ACAGAGCCTAGCGAAGGATCTGCCCCAGGATCTCCTGCCGGCTCT
CCAACATCTACCGAAGAGGGAACCAGCACCGAGCCATCTGAGGGA
TCTGCTCCCGGAACAAGCACAGAGCCTTCAGAAGGATCCGCTCCT
GGCACCTCTGAAAGCGCCACACCAGAAAGCGGACCTGGCTCTGAA
CCTGCCACAAGCGGATCTGAGACACCTGGAAGCGAGCCAGCCACA
TCTGGCAGCGAAACACCTGGATCACCAGCCGGATCTCCCACCTCT
ACCGAGGAAGGGACATCCGAGAGCGCTACCCCAGAATCTGGACCA
GGCACCAGCACAGAACCCTCTGAAGGTTCAGCCCCTGGAACCTCT
ACCGAACCATCAGAAGGCAGCGCTCCAGGTTCTCCCGCTGGATCC
CCTACATCCACAGAAGAGGGCACCTCCACTGAACCTAGCGAGGGA
AGTGCTCCCGGCACTTCCACAGAACCATCCGAGGGCAGTGCACCT
GGAACCAGCGAATCTGCTACCCCTGAGAGTGGACCCGGAACATCC
ACTGAGCCCTCCGAGGGTTCAGCTCCAGGCACATCAGAATCCGCC
ACTCCAGAGTCCGGACCAGGATCTGAGCCAGCTACCAGCGGCTCT
GAAACACCCGGCACTAGTACCGAGCCAAGCGAGGGTAGCGCACCA
GGGACAAGTACCGAACCGTCTGAGGGCTCCGCACCAGGCACTTCC
GAAAGTGCTACTCCTGAAAGCGGCCCAGGCACTAGCGAATCCGCA
ACACCCGAGAGCGGTCCTGGAAGTCCTGCAGGTTCACCTACCAGC
ACTGAAGAGGGGACTAGCGAGAGCGCAACTCCTGAATCAGGCCCT
GGATCCGAACCTGCTACCTCCGGAAGTGAAACCCCTGGGACAAGC
GAAAGTGCAACGCCCGAGTCAGGACCCGGGACTAGCACGGAACCC
AGTGAAGGATCTGCACCCGGGACATCTACCGAGCCGTCAGAAGGT
TCTGCTCCAGGGACTAGTACTGAGCCTTCCGAAGGTTCTGCACCT
GGAACTTCCACAGAGCCCAGTGAAGGCAGTGCCCCTGGCACAAGC
ACTGAACCGTCCGAAGGCAGTGCTCCCGGGACCAGTACAGAACCG
AGCGAGGGCTCTGCTCCTGGTAGTCCAGCAGGATCTCCAACTAGC
ACCGAAGAAGGGACTTCCACCGAGCCTTCCGAGGGAAGCGCTCCT
GGAACATCCGAGTCCGCTACGCCAGAGAGTGGCCCAGGTTCTGAA
CCCGCTACTTCCGGCTCAGAGACTCCTGGGACTTCTGAGTCTGCA
ACCCCGGAAAGTGGTCCTGGTAGCGAACCAGCAACTAGCGGAAGC
GAGACACCCGGAACCTCAGAGAGTGCTACACCGGAATCCGGTCCA
GGGACGTCTACGGAACCGTCTGAAGGATCAGCTCCCGGCACTAGC
GAAAGCGCTACACCTGAAAGTGGTCCCGGATCTCCAGCAGGCAGC
CCAACCTCTACTGAAGAAGGTTCCCCAGCTGGAAGCCCCACTTCC
ACTGAGGAAGGCTCTCCCGCAGGCTCACCCACTAGTACGGAAGAA
GGCACGTCCGAGTCTGCTACTCCCGAATCCGGACCTGGAACTAGC
ACTGAGCCAAGCGAAGGATCAGCACCCGGAACCTCTGAGTCCGCC
ACACCAGAATCTGGTCCTGGTTCCGAGCCTGCCACTTCAGGATCA
GAAACCCCGGGCACGAGTGAATCAGCAACGCCGGAATCTGGCCCC
GGAAGCGAACCGGCTACGTCTGGATCTGAAACGCCAGGGACCTCC
GAATCAGCTACGCCTGAGTCTGGTCCAGGGACATCCACCGAACCT
AGTGAAGGCTCCGCACCTGGAAGCCCTGCTGGAAGCCCAACGAGT
ACTGAAGAGGGCACTTCTGAGAGCGCTACGCCTGAGTCAGGACCT
GGAAGCGAACCTGCAACATCCGGCTCAGAAACACCAGGGACCAGC
GAAAGCGCAACCCCAGAGAGTGGACCTGGATCTCCAGCTGGCTCT
CCTACTAGTACAGAGGAAGGCAGCCCTGCTGGCTCCCCAACGTCA
ACAGAAGAAGGTACTAGCACAGAGCCCAGCGAGGGTTCCGCTCCG
GGAACTTCTGAATCTGCTACACCCGAGTCAGGTCCTGGTACAAGC
GAGTCAGCTACGCCCGAAAGTGGACCTGGCACCTCAGAGTCTGCA
ACTCCTGAGAGCGGTCCAGGATCAGAACCAGCCACCTCTGGCTCT
GAGACACCAGGTTCTGAGCCTGCAACGTCCGGAAGCGAAACACCA
GGCAGTCCTGCCGGAAGTCCTACTTCAACCGAAGAGGGGACCTCT
ACAGAGCCATCAGAGGGCTCTGCACCGGGCACCTCAACAGAACCA
TCTGAAGGATCCGCACCGGGCTCTGAGCCTGCTACTAGTGGAAGC
GAAACTCCTGGCACCAGTGAATCCGCTACTCCCGAGTCTGGCCCG
GGAACGTCTACTGAACCATCTGAGGGAAGTGCCCCAGGCTAA

When a variant or fragment of the leader sequence, GLP-2 receptor agonist, or fusion domain is desired, the coding sequences for these peptides may be generated using site-directed mutagenesis of the wild-type nucleic acid sequence. Alternatively or additionally, web-based or commercially available computer programs, as well as service based companies may be used to back translate the amino acids sequences to nucleic acid coding sequences, including both RNA and/or cDNA. See, e.g., backtranseq by EMBOSS, ebi.ac.uk/Tools/st/; Gene Infinity (geneinfinity.org/sms-/sms_backtranslation.html); ExPasy (expasy.org/tools/). In one embodiment, the RNA and/or cDNA coding sequences are designed for optimal expression in the subject species for which administration is ultimately intended, e.g., a human.

The coding sequences may be designed for optimal expression using codon optimization. Codon-optimized coding regions can be designed by various different methods. This optimization may be performed using methods which are available on-line, published methods, or a company which provides codon optimizing services. One codon optimizing method is described, e.g., in International Patent Application Pub. No. WO 2015/012924, which is incorporated by reference herein. Briefly, the nucleic acid sequence encoding the product is modified with synonymous codon sequences. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

In addition to the leader sequences, GLP-2 receptor agonists, fusion domains, and fusion proteins provided herein, nucleic acid sequences encoding these polypeptides are provided. In one embodiment, a nucleic acid sequence is provided which encodes for the GLP-2 peptides described herein. In some embodiments, this may include any nucleic acid sequence which encodes the GLP-2 sequence of SEQ ID NO: 1. In another embodiment, this includes any nucleic acid which includes the GLP-2 sequence of SEQ ID NO: 2.

In one embodiment, a nucleic acid sequence is provided which encodes for the GLP-2 fusion protein described herein. In another embodiment, this includes any nucleic acid sequence which encodes the GLP-2 fusion protein of SEQ ID NO: 13, 15, 17, 19, 21 or 23.

### Expression Cassettes

Provided herein, in another aspect, is an expression cassette comprising a nucleic acid encoding a GLP-2 fusion protein as described herein. As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences (also referred to as elements) that are contiguous or non-contiguous with the nucleic acid sequence and regulatory sequences that act in trans or cis nucleic acid sequence. Such regulatory sequences typically include, e.g., one or more of a promoter, an enhancer, a transcription factor, transcription terminator, an intron, sequences that enhance translation efficiency (i.e., a Kozak consensus sequence), efficient RNA processing signals such as slicing and a polyadenylation sequence, sequences that stabilize cytoplasmic mRNA, for example Woodchuck Hepatitis Virus (WHP) posttranslational Regulatory Element (WPRE), and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell.

In one embodiment, the expression cassette refers to a nucleic acid molecule which comprises the GLP-2 construct coding sequences (e.g., coding sequences for the GLP-2 fusion protein), promoter, and may include other regulatory sequences therefor, which cassette may be engineered into a genetic element and/or packaged into the capsid of a viral vector (e.g., a viral particle). Typically, such an expression cassette for generating a viral vector contains the GLP-2 construct sequences described herein flanked by packaging signals of the viral genome (and is termed a “vector genome”) and other expression control sequences such as those described herein. Any of the expression control sequences can be optimized for a specific species using techniques known in the art including, e.g., codon optimization, as described herein.

In certain embodiments, the expression cassette includes a constitutive promoter. In another embodiment, a CB7 promoter is used. CB7 is a chicken β-actin promoter with cytomegalovirus enhancer elements. In some embodiments, the CB7 promoter has the nucleic acid sequence of SEQ ID NO: 25. In one embodiment, the promoter is a CMV promoter. In some embodiments, the CMV promoter is a nucleic acid sequence of SEQ ID NO: 26.

In another embodiment, a tissue specific promoter is used. Alternatively, other liver-specific promoters may be used such as those listed in the Liver Specific Gene Promoter Database, Cold Spring Harbor, (rulai.schl.edu/LSPD), and including, but not limited to, alpha 1 anti-trypsin (A1AT); human albumin (Miyatake et al., J. Virol., 71:5124 32 (1997)), humAlb; hepatitis B virus core promoter (Sandig et al., Gene Ther., 3:1002 9 (1996)); a TTR minimal enhancer/promoter, alpha-antitrypsin promoter, liver-specific promoter (LSP) (Wu et al. Mol Ther. 16:280-289 (2008)), TBG liver specific promoter. Other promoters, such as viral promoters, constitutive promoters, regulatable promoters (see, e.g., WO 2011/126808 and WO 2013/04943), or a promoter responsive to physiologic cues may be used may be utilized in the vectors described herein.

In one embodiment, the promoter is comprised in an inducible gene expression system. The inducible gene regulation/expression system contains at least the following components: a promoter operably linked to transgene encoding the GLP-2 fusion protein described herein (also referred to as the regulatable promoter), an activation domain, DNA binding domain, and zinc finger homeodomain binding site(s). In other embodiments, additional components may be included in the expression system, as further described herein.

The system comprises the promoter upstream of the coding sequence for the GLP-2 fusion protein. Promoters described herein, such as CMV and CB7 promoters may be used. In one embodiment, the promoter is a CMV promoter, such as that shown in SEQ ID NO: 26. In another embodiment, the promoter is the ubiquitous, inducible promoter Z12I which comprises 12 repeated copies of the binding site for ZFHD1 and the IL2 minimal promoter. See, e.g., Chen et al, Hum Gene Ther Methods. 2013 August; 24(4): 270-278, which is incorporated herein.

The expression system comprises an activation domain, which is preferably located upstream of the DNA binding domain. In one embodiment, the activation domain is a fusion of the carboxy terminus from the p65 subunit of NF-kappa B and FKBP12-rapamycin binding (FRB) domain of FKBP12-rapamycin-associated protein (FRAP). In one embodiment, the activation domain is a FKBP12-rapamycin binding (FRB) domain of human FKBP12-rapamycin-associated protein (FRAP) fused to a carboxy terminus from the p65 subunit of NF-kappa B from a human.

In one embodiment, there is a linker between the transactivation domain and DNA binding domain, which linker may be an F2A or an IRES. In one embodiment the linker is selected from an IRES or a 2A peptide.

The DNA binding domain is composed of a DNA-binding fusion of zinc finger homeodomain 1 (ZFHD1) joined to up to three copies of FK506 binding protein (FKBP). In the presence of an inducing agent, e.g., a rapalog such as rapamycin, the DNA binding domain and activation domain are dimerized through interaction of their FKBP and FRB domains, leading to transcription activation of the transgene. In some embodiments, the ZFHD1 is included in frame with the GT2A or IRES.

The expression system is designed to have one, two or three copies of the FKBP sequence. These are termed herein FKBP subunits. In one embodiment, the subunits are designed to express the same protein, but to have nucleic acids which are divergent from one another in order to minimize recombination. For example, SEQ ID NO: 27 provides 3 “wobbled” coding sequences for FKBP, each of which encode the sequence shown in SEQ ID NO: 28:

GVQVETISPGDGRTFPKRGQTCVVHYTGMLEDGKKFDSSRDRNKP
FKFMLGKQEVIRGWEEGVAQMSVGQRAKLTISPDYAYGATGHPGI
IPPHATLVFDVELLKLE

The expression system further comprises zinc finger homeodomain binding sites. The nucleic acid molecule contains at least 1, 2, 3, 4, 5, 6, 7, 8, 9, 10, 11 or 12 binding sites for ZFHD. In one embodiment, the expression system contains 8 (eight) zinc finger homeodomains binding site (binding partners) (8XZFHD). However, the invention encompasses expression systems having from two to about twelve copies of the zinc finger binding site. An example of a single copy of a ZFHD binding site is: aatgatgggcgctcgagt (SEQ ID NO: 29)

In some embodiments, there is a minimal IL2 promoter downstream of the zinc finger homeodomain binding sites. An exemplary IL2 promoter is shown in SEQ ID NO: 30.

Such inducible systems are known in the art, and include, e.g., the rapamycin-inducible system described by e.g., Rivera et al, A humanized system for pharmacologic control of gene expression, Nature Medicine volume 2, pages 1028-1032 (September 1996) and Rivera et al, Long-term pharmacologically regulated expression of erythropoietin in primates following AAV-mediated gene transfer, Blood, 15 Feb. 2005, volume 105, number 4, both of which are incorporated herein by reference. In one embodiment, the inducible gene expression system comprises a CMV promoter, the activation domain is a FKBP12-rapamycin binding (FRB) domain of human FKBP12-rapamycin-associated protein (FRAP) fused to a carboxy terminus from the p65 subunit of NF-kappa B from a human, GT2A peptide, ZFHD1 DNA binding domain, three FKBP subunits, an hGH poly A, 8XZFHD, and a minimal sIL2 promoter. These sequences are in addition to the coding sequence for the GLP-2 fusion protein and optionally other regulatory sequences.

In addition to a promoter, an expression cassette and/or a vector may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), human growth hormone (hGH), SV40, rabbit β-globin (also referred to as rabbit globin polyA; RGB), modified RGB (mRGB) and TK polyA. Examples of suitable enhancers include, e.g., the alpha fetoprotein enhancer, the TTR minimal promoter/enhancer, LSP (TH-binding globulin promoter/alphal-microglobulin/bikunin enhancer), amongst others. In one embodiment, the polyA is a rabbit globin polyA.

These control sequences are “operably linked” to the GLP-2 construct sequences. As used herein, the term “operably linked” refers to both expression control sequences that are contiguous with the gene of interest and expression control sequences that act in trans or at a distance to control the gene of interest.

In one embodiment, a rAAV is provided which includes a 5′ ITR, CB7 promoter, chicken beta-actin intron, coding sequence for the fusion protein of SEQ ID NO: 13, 15, 17, 19, 21, or 23, a rabbit globin poly A, and a 3′ ITR. In another embodiment, the rAAV comprises a polynucleotide comprising a CMV promoter, the activation domain is a FKBP12-rapamycin binding (FRB) domain of human FKBP12-rapamycin-associated protein (FRAP) fused to a carboxy terminus from the p65 subunit of NF-kappa B from a human, IRES, ZFHD1 DNA binding domain, three FKBP subunits, an hGH poly A, SXZFHD, a minimal sIL2 promoter, coding sequence for the GLP-2 fusion protein of SEQ ID NO: 13, 15, 17, 19, 21, or 23, and rabbit beta globin polyA.

In another embodiment, a two vector inducible system is provided. The first rAAV comprises t2XZFHD, a minimal IL2 promoter, coding sequence for the GLP-2 fusion protein of SEQ ID NO: 13, 15, 17, 19, 21, or 23, and rabbit beta globin polyA. A stuffer sequence may be included to increase the packaging size of the vector. The second rAAV comprises a polynucleotide comprising a CMV promoter, an intron, the activation domain is a FKBP12-rapamycin binding (FRB) domain of human FKBP12-rapamycin-associated protein (FRAP) fused to a carboxy terminus from the p65 subunit of NF-kappa B from a human, IRES, ZFHD1 DNA binding domain, and a polyA.

In one embodiment, an expression cassette is provided that includes a polynucleotide comprising a CB7 promoter, chicken beta-actin intron, coding sequence for the fusion protein of SEQ ID NO: 13, 15, 17, 19, 21, or 23, and a rabbit globin poly A. In one embodiment, the expression cassette is that found in SEQ ID NO: X, or a sequence sharing at least 70%, 75%, 80%, 85%, 90%, 95%, 99% or 100% identity therewith. In another embodiment, a vector genome is provided wherein SEQ ID NO: X-X, or a sequence sharing at least 70%, 75%, 80%, 85%, 90%, 95%, 99% or 100% identity therewith is flanked by 5′ and 3′ AAV ITRs.

In another embodiment, an expression cassette is provided that includes a polynucleotide comprising a CB7 promoter, chicken beta-actin intron, coding sequence for the fusion protein of SEQ ID NO: 13, 15, 17, 19, 21, or 23, and a rabbit globin poly A.

### Viral Vectors

In another aspect, viral vectors that include the expression cassettes described herein are provided. In certain embodiments of the viral vectors described herein, the viral vector is an adeno-associated virus (AAV) viral vector or recombinant AAV (rAAV). The term “recombinant AAV” or “rAAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV DNase-resistant particle having an AAV protein capsid into which is packaged an expression cassette flanked by AAV inverted terminal repeat sequences (ITRs) (together referred to as the “vector genome”) for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. In one embodiment, the AAV capsid is an AAVrh91 capsid or variant thereof. In certain embodiments, the capsid protein is designated by a number or a combination of numbers and letters following the term “AAV” in the name of the rAAV vector. Unless otherwise specified, the AAV capsid, ITRs, and other selected AAV components described herein, may be readily selected from among any AAV, including, without limitation, the AAVs identified as AAV1, AAV2, AAV3, AAV4, AAV5, AAV6, AAV7, AAV8, AAV9, AAVrh10, AAVhu37, AAVrh32.33, AAVAnc80, AAV10, AAV11, AAV12, AAVrh8, AAVrh74, AAV-DJ8, AAV-DJ, AAVhu.37, AAVrh.64R1, and AAVhu68. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449, 7,282,199 (AAV8), WO 2005/033321 and U.S. Pat. No. 7,906,111 (AAV9), and WO 2006/110689, and WO 2003/042397 (rh.10), WO 2005/033321, WO 2018/160582 (AAVhu68), which are incorporated herein by reference. Other suitable AAVs may include, without limitation, AAVrh90 [PCT/US20/30273, filed Apr. 28, 2020], AAVrh91 [PCT/US20/030266, filed Apr. 28, 2020, now a publication WO 2020/223231, published Nov. 5, 2020], AAVrh92, AAVrh93, AAVrh91.93 [PCT/US20/30281, filed Apr. 28, 2020], which are incorporated by reference herein. Other suitable AAV include AAV3B variants which are described in U.S. Provisional Patent Application No. 62/924,112, filed Oct. 21, 2019, and U.S. Provisional Patent Application No. 63/025,753, filed May 15, 2020, describing AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17, which are incorporated herein by reference. See also, International Patent Application No. PCT/US21/45945, filed Aug. 13, 2021, U.S. Provisional Patent Application No. 63/065,616, filed Aug. 14, 2020, and U.S. Provisional Patent Application No. 63/109,734, filed Nov. 4, 2020, which are all incorporated herein by reference in its entireties. These documents also describe other AAV capsids which may be selected for generating rAAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models.

As used herein, relating to AAV, the term “variant” means any AAV sequence which is derived from a known AAV sequence, including those with a conservative amino acid replacement, and those sharing at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3).

In one embodiment, the viral vector is an rAAV having the capsid of AAV8 or a functional variant thereof. In one embodiment, the viral vector is an rAAV having the capsid of AAVrh91 or a functional variant thereof. In one embodiment, the viral vector is an rAAV having the capsid of AAV3.AR.2.12 or a functional variant thereof. In one embodiment, the viral vector is an rAAV having a capsid selected from AAV9, AAVrh64R1, AAVhu37, or AAVrh10.

In certain embodiments, a novel isolated AAVrh91 capsid is provided. A nucleic acid sequence encoding the AAVrh91 capsid is provided in SEQ ID NO: 31 and the encoded amino acid sequence is provided in SEQ ID NO: 32. Provided herein is an rAAV comprising at least one of the vp1, vp2 and the vp3 of AAVrh91 (SEQ ID NO: 32). Also provided herein are rAAV comprising an AAV capsid encoded by at least one of the vp1, vp2 and the vp3 of AAVrh91 (SEQ ID NO: 31). In yet another embodiment, a nucleic acid sequence encoding the AAVrh91 amino acid sequence is provided in SEQ ID NO: 19 and the encoded amino acid sequence is provided in SEQ ID NO: 32. Also provided herein are rAAV comprising an AAV capsid encoded by at least one of the vp1, vp2 and the vp3 of AAVrh91eng (SEQ ID NO: 19). In certain embodiments, the vp1, vp2 and/or vp3 is the full-length capsid protein of AAVrh91 (SEQ ID NO: 32). In other embodiments, the vp1, vp2 and/or vp3 has an N-terminal and/or a C-terminal truncation (e.g., truncation(s) of about 1 to about 10 amino acids).

In certain embodiments, an AAVrh91 capsid is characterized by one or more of the following: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 32, vp1 proteins produced from SEQ ID NO: 31, or vp1 proteins produced from a nucleic acid sequence at least 70% identical to SEQ ID NO: 31 which encodes the predicted amino acid sequence of 1 to 736 of SEQ ID NO: 32, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 32, vp2 proteins produced from a sequence comprising at least nucleotides 412 to 2208 of SEQ ID NO: 31, or vp2 proteins produced from a nucleic acid sequence at least 70% identical to at least nucleotides 412 to 2208 of SEQ ID NO: 31 which encodes the predicted amino acid sequence of at least about amino acids 138 to 736 of SEQ ID NO: 32, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 32, vp3 proteins produced from a sequence comprising at least nucleotides 607 to 2208 of SEQ ID NO: 31, or vp3 proteins produced from a nucleic acid sequence at least 70% identical to at least nucleotides 607 to 2208 of SEQ ID NO: 31 which encodes the predicted amino acid sequence of at least about amino acids 203 to 736 of SEQ ID NO: 32; and/or (2) a heterogeneous population of vp1 proteins which are the product of a nucleic acid sequence encoding the amino acid sequence of SEQ ID NO: 32, a heterogeneous population of vp2 proteins which are the product of a nucleic acid sequence encoding the amino acid sequence of at least about amino acids 138 to 736 of SEQ ID NO: 32, and a heterogeneous population of vp3 proteins which are the product of a nucleic acid sequence encoding at least amino acids 203 to 736 of SEQ ID NO: 32, wherein: the vp1, vp2 and vp3 proteins contain subpopulations with amino acid modifications comprising at least two highly deamidated asparagines (N) in asparagine—glycine pairs in SEQ ID NO: 32 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAVrh91 capsid is characterized by one or more of the following: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 32, vp1 proteins produced from SEQ ID NO: 19, or vp1 proteins produced from a nucleic acid sequence at least 70% identical to SEQ ID NO: 19 which encodes the predicted amino acid sequence of 1 to 736 of SEQ ID NO: 32, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 32, vp2 proteins produced from a sequence comprising at least nucleotides 412 to 2208 of SEQ ID NO: 19, or vp2 proteins produced from a nucleic acid sequence at least 70% identical to at least nucleotides 412 to 2208 of SEQ ID NO: 19 which encodes the predicted amino acid sequence of at least about amino acids 138 to 736 of SEQ ID NO: 32, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 32, vp3 proteins produced from a sequence comprising at least nucleotides 607 to 2208 of SEQ ID NO: 19, or vp3 proteins produced from a nucleic acid sequence at least 70% identical to at least nucleotides 607 to 2208 of SEQ ID NO: 19 which encodes the predicted amino acid sequence of at least about amino acids 203 to 736 of SEQ ID NO: 32; and/or (2) a heterogeneous population of vp1 proteins which are the product of a nucleic acid sequence encoding the amino acid sequence of SEQ ID NO: 32, a heterogeneous population of vp2 proteins which are the product of a nucleic acid sequence encoding the amino acid sequence of at least about amino acids 138 to 736 of SEQ ID NO: 32, and a heterogeneous population of vp3 proteins which are the product of a nucleic acid sequence encoding at least amino acids 203 to 736 of SEQ ID NO: 32, wherein: the vp1, vp2 and vp3 proteins contain subpopulations with amino acid modifications comprising at least two highly deamidated asparagines (N) in asparagine—glycine pairs in SEQ ID NO: 32 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, the AAVrh91 vp1, vp2 and vp3 proteins contain subpopulations with amino acid modifications comprising at least two highly deamidated asparagines (N) in asparagine—glycine pairs in SEQ ID NO: 32 and optionally further comprising subpopulations comprising other deamidated amino acids, wherein the deamidation results in an amino acid change. High levels of deamidation at N-G pairs N57, N383 and/or N512 are observed, relative to the number of SEQ ID NO: 32. Deamidation has been observed in other residues. In certain embodiments, AAVrh91 may have other residues deamidated, e.g., typically at less than 10% and/or may have other modifications, including phosphorylation (e.g., where present, in the range of about 2 to about 30%, or about 2 to about 20%, or about 2 to about 10%) (e.g., at S149), or oxidation (e.g., at one or more of ˜W22, ˜M211, W247, M403, M435, M471, W478, W503, ˜M537, ˜M541, ˜M559, ˜M599, M635, and/or, W695). Optionally the W may oxidize to kynurenine.

TABLE A
AAVrh91 Deamidation

| AAVrh91 | |
|---|---|
| Deamidation based | |
| on VP1 numbering | % Deamidation |
| N57 + Deamidation | 65-90, 70-95, 80-95, 75-100, 80-100, or 90-100 |
| N94 + Deamidation | 2-15 or 2-5 |
| N303 + Deamidation | 2-15 or 5-10 |
| N383 + Deamidation | 65-90, 70-95, 80-95, 75-100, 80-100, or 90-100 |
| N497 + Deamidation | 2-15 or 5-10 |
| N512 + Deamidation | 65-90, 70-95, 80-95, 75-100, 80-100, or 90-100 |
| ~N691 + Deamidation | 2-15, 2-10, or 5-10 |

In certain embodiments, an AAVrh91 capsid is modified in one or more of the positions identified in the preceding table, in the ranges provided, as determined using mass spectrometry with a trypsin enzyme. In certain embodiments, one or more of the positions, or the glycine following the N is modified as described herein. Residue numbers are based on the AAVrh91 sequence provided herein. See, SEQ ID NO: 32.

In certain embodiments, an AAVrh91 capsid comprises: a heterogeneous population of vp1 proteins which are the product of a nucleic acid sequence encoding the amino acid sequence of SEQ ID NO: 32, a heterogeneous population of vp2 proteins which are the product of a nucleic acid sequence encoding the amino acid sequence of at least about amino acids 138 to 736 of SEQ ID NO: 32, and a heterogeneous population of vp3 proteins which are the product of a nucleic acid sequence encoding at least amino acids 203 to 736 of SEQ ID NO: 32.

In certain embodiments, the modified AAVrh91 nucleic acid sequences is be used to generate a mutant rAAV having a capsid with lower deamidation than the native AAVrh91 capsid. Such mutant rAAV may have reduced immunogenicity and/or increase stability on storage, particularly storage in suspension form.

In one aspect, a recombinant AAV (rAAV) is provided. The rAAV includes an AAV capsid from adeno-associated virus rh91, and a vector genome packaged in the AAV capsid, said vector genome comprising AAV inverted terminal repeats (ITRs), a coding sequence for the GLP-2 receptor agonist of SEQ ID NO: 14, and regulatory sequences which direct expression of the GLP-2 receptor agonist.

In certain embodiments, an AAV68 capsid is further characterized by one or more of the following. AAV hu68 capsid proteins comprise: AAVhu68 vp1 proteins produced by expression from a nucleic acid sequence which encodes the predicted amino acid sequence of 1 to 736 of SEQ ID NO: 55, vp1 proteins produced from SEQ ID NO: 53 or 54, or vp1 proteins produced from a nucleic acid sequence at least 70% identical to SEQ ID NO: 53 or 54 which encodes the predicted amino acid sequence of 1 to 736 of SEQ ID NO: 55; AAVhu68 vp2 proteins produced by expression from a nucleic acid sequence which encodes the predicted amino acid sequence of at least about amino acids 138 to 736 of SEQ ID NO: 55, vp2 proteins produced from a sequence comprising at least nucleotides 412 to 2211 of SEQ ID NO: 53 or 54, or vp2 proteins produced from a nucleic acid sequence at least 70% identical to at least nucleotides 412 to 2211 of SEQ ID NO: 53 or 54 which encodes the predicted amino acid sequence of at least about amino acids 138 to 736 of SEQ ID NO: 55, and/or AAVhu68 vp3 proteins produced by expression from a nucleic acid sequence which encodes the predicted amino acid sequence of at least about amino acids 203 to 736 of SEQ ID NO: 55, vp3 proteins produced from a sequence comprising at least nucleotides 607 to 2211 of SEQ ID NO: 53 or 54, or vp3 proteins produced from a nucleic acid sequence at least 70% identical to at least nucleotides 607 to 2211 of SEQ ID NO: 53 or 54 which encodes the predicted amino acid sequence of at least about amino acids 203 to 736 of SEQ ID NO: 55.

Additionally or alternatively, an AAV capsid is provided which comprise a heterogenous population of vp1 proteins optionally comprising a valine at position 157, a heterogenous population of vp2 proteins optionally comprising a valine at position 157, and a heterogenous population of vp3 proteins, wherein at least a subpopulation of the vp1 and vp2 proteins comprise a valine at position 157 and optionally further comprising a glutamic acid at position 67 based on the numbering of the vp1 capsid of SEQ ID NO: 55. Additionally or alternatively, an AAVhu68 capsid is provided which comprises a heterogenous population of vp1 proteins which are the product of a nucleic acid sequence encoding the amino acid sequence of SEQ ID NO: 55, a heterogenous population of vp2 proteins which are the product of a nucleic acid sequence encoding the amino acid sequence of at least about amino acids 138 to 736 of SEQ ID NO: 55, and a heterogenous population of vp3 proteins which are the product of a nucleic acid sequence encoding at least amino acids 203 to 736 of SEQ ID NO: 55, wherein: the vp1, vp2 and vp3 proteins contain subpopulations with amino acid modifications

The AAVhu68 vp1, vp2 and vp3 proteins are typically expressed as alternative splice variants encoded by the same nucleic acid sequence which encodes the full-length vp1 amino acid sequence of SEQ ID NO: 55 (amino acid 1 to 736). Optionally the vp1-encoding sequence is used alone to express the vp1, vp2 and vp3 proteins. Alternatively, this sequence may be co-expressed with one or more of a nucleic acid sequence which encodes the AAVhu68 vp3 amino acid sequence of SEQ ID NO: 55 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 53 or 54), or a sequence at least 70% to at least 99% (e.g., at least 85%, at least 90%, at least 95%, at least 97%, at least 98% or at least 99%) identical to SEQ ID NO: 53 or 54 which encodes aa 203 to 736 of SEQ ID NO: 55. Additionally, or alternatively, the vp1-encoding and/or the vp2-encoding sequence may be co-expressed with the nucleic acid sequence which encodes the AAVhu68 vp2 amino acid sequence of SEQ ID NO: 55 (about aa 138 to 736) without the vp1-unique region (about aa 1 to about 137), or a strand complementary thereto, the corresponding mRNA or tRNA (nt 412 to 2212 of SEQ ID NO: 53 or 54), or a sequence at least 70% to at least 99% (e.g., at least 85%, at least 90%, at least 95%, at least 97%, at least 98% or at least 99%) identical to SEQ ID NO: 53 or 54 which encodes about aa 138 to 736 of SEQ ID NO: 55.

As described herein, a rAAVhu68 has a rAAVhu68 capsid produced in a production system expressing capsids from an AAVhu68 nucleic acid which encodes the vp1 amino acid sequence of SEQ ID NO: 55, and optionally additional nucleic acid sequences, e.g., encoding a vp3 protein free of the vp1 and/or vp2-unique regions. The rAAVhu68 resulting from production using a single nucleic acid sequence vp1 produces the heterogenous populations of vp1 proteins, vp2 proteins and vp3 proteins. More particularly, the AAVhu68 capsid contains subpopulations within the vp1 proteins, within the vp2 proteins and within the vp3 proteins which have modifications from the predicted amino acid residues in SEQ ID NO: 55. These subpopulations include, at a minimum, deamidated asparagine (N or Asn) residues. For example, asparagines in asparagine—glycine pairs are highly deamidated.

In one embodiment, the AAVhu68 vp1 nucleic acid sequence has the sequence of SEQ ID NO: 53 or 54, or a strand complementary thereto, e.g., the corresponding mRNA or tRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 55 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 53 or 54). In certain embodiments, also provided is a nucleic acid sequence which encodes the AAVhu68 vp2 amino acid sequence of SEQ ID NO: 55 (about aa 138 to 736) without the vp1-unique region (about aa 1 to about 137), or a strand complementary thereto, the corresponding mRNA or tRNA (nt 412 to 2211 of SEQ ID NO: 53 or 54).

However, other nucleic acid sequences which encode the amino acid sequence of SEQ ID NO: 55 may be selected for use in producing rAAVhu68 capsids. In certain embodiments, the nucleic acid sequence has the nucleic acid sequence of SEQ ID NO: 53 or 54 or a sequence at least 70% to 99% identical, at least 75%, at least 80%, at least 85%, at least 90%, at least 95%, at least 97%, at least 99%, identical to SEQ ID NO: 53 or 54 which encodes SEQ ID NO: 55. In certain embodiments, the nucleic acid sequence has the nucleic acid sequence of SEQ ID NO: 53 or 54 or a sequence at least 70% to 99.%, at least 75%, at least 80%, at least 85%, at least 90%, at least 95%, at least 97%, at least 99%, identical to about nt 412 to about nt 2211 of SEQ ID NO: 53 or 54 which encodes the vp2 capsid protein (about aa 138 to 736) of SEQ ID NO: 55. In certain embodiments, the nucleic acid sequence has the nucleic acid sequence of about nt 607 to about nt 2211 of SEQ ID NO: 53 or 54 or a sequence at least 70% to 99.%, at least 75%, at least 80%, at least 85%, at least 90%, at least 95%, at least 97%, at least 99%, identical to nt SEQ ID NO: 53 or 54 which encodes the vp3 capsid protein (about aa 203 to 736) of SEQ ID NO: 55.

| Deamidation | | |
|---|---|---|
| Based on Predicted | Average % Based on VP1/VP2/VP3 | |
| AAVHu68 [SEQ ID NO: 55] | Proteins in AAVhu68 Capsid | |
| Deamidated Residue + 1 | Broad Range of | |
| (Neighboring AA) | Percentages (%) | Narrow Ranges (%) |
| N57 | 78 to 100% | 80 to 100, 85 to 97 |
| (N-G) | | |
| N66 | 0 to 5 | 0, 1 to 5 |
| (N-E) | | |
| N94 | 0 to 15, | 0, 1 to 15, 5 to 12, 8 |
| (N-H) | | |
| N113 | 0 to 2 | 0, 1 to 2 |
| (N-L) | | |
| ~N253 | 10 to 25 | 15 to 22 |
| (N-N) | | |
| Q259 | 8 to 42 | 10 to 40, 20 to 35 |
| (Q-I) | | |
| ~N270 | 12 to 30 | 15 to 28 |
| (N-D) | | |
| ~N304 | 0 to 5 | 1 to 4 |
| (N-N) | | |
| (position 303 also N) | | |
| N319 | 0 to 5 | 0, 1 to 5, 1 to 3 |
| (N-I) | | |
| N329 * | 65 to 100 | 70 to 95, 85 to 95, |
| (N-G)* | | 80 to 100, 85 to 100, |
| (position 328 also N) | | |
| N336 | 0 to 100 | 0, 1 to 10, 25 to 100, |
| (N-N) | | 30 to 100, 30 to 95 |
| ~N409 | 15 to 30 | 20 to 25 |
| (N-N) | | |
| N452 | 75 to 100 | 80 to 100, 90 to 100, |
| (N-G) | | 95 to 100, |
| N477 | 0 to 8 | 0, 1 to 5 |
| (N-Y) | | |
| N512 | 65 to 100 | 70 to 95, 85 to 95, |
| (N-G) | | 80 to 100, 85 to 100, |
| ~N515 | 0 to 25 | 0, 1 to 10, 5 to 25, |
| (N-S) | | 15 to 25 |
| ~Q599 | 1 to 20 | 2 to 20, 5 to 15 |
| (Asn-Q-Gly) | | |
| N628 | 0 to 10 | 0, 1 to 10, 2 to 8 |
| (N-F) | | |
| N651 | 0 to 3 | 0, 1 to 3 |
| (N-T) | | |
| N663 | 0 to 5 | 0, 1 to 5, 2 to 4 |
| (N-K) | | |
| N709 | 0 to 25 | 0,1 to 22, 15 to 25 |
| (N-N) | | |
| N735 | 0 to 40 | 0. 1 to 35, 5 to 50, |
| | | 20 to 35 |

In certain embodiments, the AAVhu68 capsid is characterized, by having, capsid proteins in which at least 45% of N residues are deamidated at least one of positions N57, N329, N452, and/or N512 based on the numbering of amino acid sequence of SEQ ID NO: 55. In certain embodiments, at least about 60%, at least about 70%, at least about 80%, or at least 90% of the N residues at one or more of these N-G positions (i.e., N57, N329, N452, and/or N512, based on the numbering of amino acid sequence of SEQ ID NO: 55) are deamidated. In these and other embodiments, an AAVhu68 capsid is further characterized by having a population of proteins in which about 1% to about 20% of the N residues have deamidations at one or more of positions: N94, N253, N270, N304, N409, N477, and/or Q599, based on the numbering of amino acid sequence of SEQ ID NO: 55. In certain embodiments, the AAVhu68 comprises at least a subpopulation of vp1, vp2 and/or vp3 proteins which are deamidated at one or more of positions N35, N57, N66, N94, N113, N252, N253, Q259, N270, N303, N304, N305, N319, N328, N329, N336, N409, N410, N452, N477, N515, N598, Q599, N628, N651, N663, N709, N735, based on the numbering of amino acid sequence of SEQ ID NO: 55, or combinations thereof. In certain embodiments, the capsid proteins may have one or more amidated amino acids.

In another embodiment a recombinant adeno-associated virus (rAAV) is provided that has an AAVhu68 capsid and a vector genome, wherein (a) the AAV hu68 capsid comprises a heterogenous population of AAVhu68 vp1 proteins, a heterogenous population of AAVhu68 vp2 proteins; and a heterogenous population of AAVhu68 vp3 proteins, wherein the heterogenous AAVhu68 vp1, AAVhu68 vp2 and AAVhu68 vp3 proteins contain subpopulations with amino acid modifications comprising 50% to 100% deamidation in at least two asparagines (N) in asparagine—glycine pairs in two or more of N57, N329, N452, N512 of SEQ ID NO: 55 as determined using mass spectrometry and optionally further comprising subpopulations comprising other deamidated amino acids, wherein the deamidation results in an amino acid change, wherein the deamidated asparagines are deamidated to aspartic acid, isoaspartic acid, an interconverting aspartic acid/isoaspartic acid pair, or combinations thereof, wherein the AAVhu68 capsid further comprises subpopulations having one or more of:
at least 65% of asparagines (N) in asparagine—glycine pairs located at positions N57 of the vp1 proteins are deamidated, based on the numbering of SEQ ID NO: 55;
at least 75% of N in asparagine—glycine pairs in position N329 of the vp1, v2 and vp3 proteins are deamidated, based on the residue numbering of the amino acid sequence of SEQ ID NO: 55,
at least 50% of N in asparagine—glycine pairs in position N452 of the vp1, v2 and vp3 proteins are deamidated, based on the residue numbering of the amino acid sequence of SEQ ID NO: 55; and/or
at least 75% of N in asparagine—glycine pairs in position N512 of the vp1, v2 and vp3 proteins are deamidated, based on the residue numbering of the amino acid sequence of SEQ ID NO: 55, and
a vector genome in the AAVhu68 capsid, the vector genome comprising a nucleic acid molecule comprising AAV inverted terminal repeat sequences and a non-AAV nucleic acid sequence encoding a GLP-2 fusion as described herein operably linked to sequences which direct expression of GLP-2 fusion in a target cell.

In one embodiment, the rAAV is an scAAV. The abbreviation “sc” refers to self-complementary. “Self-complementary AAV” refers a plasmid or vector having an expression cassette in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

In one embodiment, the nucleic acid sequences encoding the GLP-2 constructs described herein are engineered into any suitable genetic element, e.g., naked DNA, phage, transposon, cosmid, RNA molecule (e.g., mRNA), episome, etc., which transfers the GLP-2 sequences carried thereon to a host cell, e.g., for generating nanoparticles carrying DNA or RNA, viral vectors in a packaging host cell and/or for delivery to a host cell in a subject. In one embodiment, the genetic element is a plasmid. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

As used herein, the term “host cell” may refer to the packaging cell line in which a vector (e.g., a recombinant AAV or rAAV) is produced from a production plasmid. In the alternative, the term “host cell” may refer to any target cell in which expression of a gene product described herein is desired. Thus, a “host cell,” refers to a prokaryotic or eukaryotic cell (e.g., bacterial cell, human cell or insect cell) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species for in vitro assessment of the compositions described herein. In other embodiments herein, the term “host cell” refers to the cells employed to generate and package the viral vector or recombinant virus. In a further embodiment, the term “host cell” is an intestine cell, a small intestine cell, a pancreatic cell, a liver cell.

As used herein, the term “target cell” refers to any target cell in which expression of a heterologous nucleic acid sequence or protein is desired. In certain embodiments, the target cell is a liver cell. In other embodiments, the target cell is a muscle cell.

In one embodiment, the rAAV is provided which comprises a vector genome comprising an expression cassette, wherein the expression cassette comprises a CMV promoter, the activation domain is a FKBP12-rapamycin binding (FRB) domain of human FKBP12-rapamycin-associated protein (FRAP) fused to a carboxy terminus from the p65 subunit of NF-kappa B from a human, GT2A_V1 peptide, ZFHD1 DNA binding domain, three FKBP subunits, an hGH poly A, 12XZFHD, a minimal sIL2 promoter, coding sequence for the GLP-2 fusion protein of SEQ ID NO: 13, 15, 17, 19, 21, or 23, and rabbit beta globin polyA. In another embodiment, the rAAV is provide which comprises a vector genome comprising an expression cassette, wherein the expression cassette comprises a CMV promoter, the activation domain is a FKBP12-rapamycin binding (FRB) domain of human FKBP12-rapamycin-associated protein (FRAP) fused to a carboxy terminus from the p65 subunit of NF-kappa B from a human, GT2A_V2 peptide, ZFHD1 DNA binding domain, three FKBP subunits, an hGH poly A, 12XZFHD, a minimal sIL2 promoter, coding sequence for the GLP-2 fusion protein of SEQ ID NO: 13, 15, 17, 19, 21, or 23, and rabbit beta globin polyA.

In one embodiment, the rAAV is provided which comprises a vector genome comprising an expression cassette, wherein the expression cassette comprises a CB7 promoter, chicken beta-actin intron, coding sequence for the fusion protein of SEQ ID NO: 13, 15, 17, 19, 21, or 23, a rabbit globin poly A, and a 3′ ITR. In another embodiment, the rAAV is provided which comprises a vector genome comprising an expression cassette, wherein the expression cassette comprises a CMV promoter, the activation domain is a FKBP12-rapamycin binding (FRB) domain of human FKBP12-rapamycin-associated protein (FRAP) fused to a carboxy terminus from the p65 subunit of NF-kappa B from a human, IRES, ZFHD1 DNA binding domain, three FKBP subunits, an hGH poly A, 8XZFHD, a minimal sIL2 promoter, coding sequence for the GLP-2 fusion protein of SEQ ID NO: 13, 15, 17, 19, 21, or 23 and rabbit beta globin polyA.

In another embodiment, a two vector inducible system is provided. In one embodiment, the first rAAV is provided which comprises a vector genome comprising an expression cassette, wherein the expression cassette comprises a 12XZFHD, a minimal IL2 promoter, coding sequence for the GLP-2 fusion protein of SEQ ID NO: 13, 15, 17, 19, 21, or 23, and rabbit beta globin polyA. A stuffer sequence may be included to increase the packaging size of the vector. The second rAAV comprises a vector genome comprising an expression cassette, wherein the expression cassette comprises a polynucleotide comprising a CMV promoter, an intron, the activation domain is a FKBP12-rapamycin binding (FRB) domain of human FKBP12-rapamycin-associated protein (FRAP) fused to a carboxy terminus from the p65 subunit of NF-kappa B from a human, IRES, ZFHD1 DNA binding domain, and a polyA.

In one embodiment, an rAAV includes a vector genome that includes an expression cassette that includes a polynucleotide comprising a CB7 promoter, chicken beta-actin intron, coding sequence for the fusion protein of SEQ ID NO: 13, 15, 17, 19, 21, or 23, and a rabbit globin poly A.

The minimal sequences required to package the expression cassette into an AAV viral particle are the AAV 5′ and 3′ ITRs, which may be of the same AAV origin as the capsid, or of a different AAV origin (to produce an AAV pseudotype). In one embodiment, the ITR sequences from AAV2, or the deleted version thereof (AITR), are used for convenience and to accelerate regulatory approval. However, ITRs from other AAV sources may be selected.

Preferably, the source of the ITRs is the same as the source of the Rep protein, which is provided in trans for production. Typically, an expression cassette for an AAV vector comprises an AAV 5′ ITR, the GLP-2 fusion protein coding sequences and any regulatory sequences, and an AAV 3′ ITR. However, other configurations of these elements may be suitable. A shortened version of the 5′ ITR, termed AITR, has been described in which the D-sequence and terminal resolution site (trs) are deleted. In other embodiments, the full-length AAV 5′ and 3′ ITRs are used.

For packaging an expression cassette into virions, the ITRs are the only AAV components required in cis in the same construct as the gene. In one embodiment, the coding sequences for the replication (rep) and/or capsid (cap) are removed from the AAV genome and supplied in trans or by a packaging cell line in order to generate the AAV vector. For example, as described above, a pseudotyped AAV may contain ITRs from a source which differs from the source of the AAV capsid. In one embodiment, a chimeric AAV capsid may be utilized. Still other AAV components may be selected. Sources of such AAV sequences are described herein and may also be isolated or obtained from academic, commercial, or public sources (e.g., the American Type Culture Collection, Manassas, VA). The AAV sequences may be obtained through synthetic or other suitable means by reference to published sequences such as are available in the literature or in databases such as, e.g., GenBank®, PubMed®, or the like.

Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. The methods used to construct any embodiment of this invention are known to those with skill in nucleic acid manipulation and include genetic engineering, recombinant engineering, and synthetic techniques. See, e.g., Green and Sambrook et al, Molecular Cloning: A Laboratory Manual, Cold Spring Harbor Press, Cold Spring Harbor, NY (2012). Similarly, methods of generating rAAV virions are well known and the selection of a suitable method is not a limitation on the present invention. See, e.g., K. Fisher et al, (1993) J. Virol., 70:520-532 and U.S. Pat. No. 5,478,745.

The rAAV described herein comprise a selected capsid with a vector genome packaged inside. The vector genome (or rAAV genome) comprises 5′ and 3′ AAV inverted terminal repeats (ITRs), the polynucleotide sequence encoding the fusion protein, and regulatory sequences which direct insertion of the polynucleotide sequence encoding the fusion protein to the genome of a host cell. In one embodiment, the vector genome is the sequence shown in SEQ ID NO: 16 or a sequence sharing at least 70%, at least 75%, at least 80%, at least 85%, at least 90%, at least 95%, or at least 99% identity therewith.

As used herein, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s) (i.e., transgene(s)), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue. Suitable components of a vector genome are discussed in more detail herein. In one example, a “vector genome” contains, at a minimum, from 5′ to 3′, a vector-specific sequence, a nucleic acid sequence encoding GLP-2 constructs operably linked to regulatory control sequences (which direct their expression in a target cell), where the vector-specific sequence may be a terminal repeat sequence which specifically packages the vector genome into a viral vector capsid or envelope protein. For example, AAV inverted terminal repeats are utilized for packaging into AAV and certain other parvovirus capsids.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences from AAV2. However, ITRs from other AAV sources may be selected. A shortened version of the 5′ ITR, termed AITR, has been described in which the D-sequence and terminal resolution site (trs) are deleted. In certain embodiments, the vector genome includes a shortened AAV2 ITR of 130 base pairs, wherein the external A elements is deleted. Without wishing to be bound by theory, it is believed that the shortened ITR reverts back to the wild-type length of 145 base pairs during vector DNA amplification using the internal (A′) element as a template. In other embodiments, full-length AAV 5′ and 3′ ITRs are used. Where the source of the ITRs is from AAV2 and the AAV capsid is from another AAV source, the resulting vector may be termed pseudotyped. However, other configurations of these elements may be suitable.

Optionally, the GLP-2 constructs described herein may be delivered via viral vectors other than rAAV. Such other viral vectors may include any virus suitable for gene therapy, including but not limited to adenovirus; herpes virus; lentivirus; retrovirus; etc. Suitably, where one of these other vectors is generated, it is produced as a replication-defective viral vector.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

Also provided are compositions which include the viral vector constructs described herein. The pharmaceutical compositions described herein are designed for delivery to subjects in need thereof by any suitable route or a combination of different routes. Direct delivery to the liver (optionally via intravenous, via the hepatic artery, or by transplant), oral, inhalation, intranasal, intratracheal, intraarterial, intraocular, intravenous, intramuscular, subcutaneous, intradermal, and other parental routes of administration. The viral vectors described herein may be delivered in a single composition or multiple compositions. Optionally, two or more different AAV may be delivered, or multiple viruses [see, e.g., WO 2011/126808 and WO 2013/049493]. In another embodiment, multiple viruses may contain different replication-defective viruses (e.g., AAV and adenovirus). In one embodiment, administration is intramuscular. In another embodiment, administration is intravenous.

The replication-defective viruses can be formulated with a physiologically acceptable carrier for use in gene transfer and gene therapy applications. In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The nuclease resistant particles are then subjected to heat treatment to release the genome from the capsid. The released genomes are then quantitated by real-time PCR using primer/probe sets targeting specific region of the viral genome (usually poly A signal). Another suitable method for determining genome copies is quantitative—PCR (qPCR), particularly the optimized qPCR or digital droplet PCR [Lock Martin, et al, Human Gene Therapy Methods. April 2014, 25(2): 115-125. doi:10.1089/hgtb.2013.131, published online ahead of editing Dec. 13, 2013].

Also, the replication-defective virus compositions can be formulated in dosage units to contain an amount of replication-defective virus that is in the range of about 1.0×10⁹ GC to about 1.0×10¹⁵ GC. In another embodiment, this amount of viral genome may be delivered in split doses. In one embodiment, the dose is about 1.0×10¹⁰ GC to about 3.0×10¹⁴ GC for an average human subject of about 70 kg. In another embodiment, the dose about 1×10⁹ GC. For example, the dose of AAV virus may be about 1×10¹⁰ GC, 1×10¹¹ GC, about 5×10¹¹ GC, about 1×10¹² GC, about 5×10¹² GC, or about 1×10¹³ GC. In another embodiment, the dosage is about 1.0×10⁹ GC/kg to about 3.0×10¹⁴ GC/kg for a human subject. In another embodiment, the dose about 1×10⁹ GC/kg. For example, the dose of AAV virus may be about 1×10¹⁰ GC/kg, 1×10¹¹ GC/kg, about 5×10¹¹ GC/kg, about 1×10¹² GC/kg, about 5×10¹² GC/kg, or about 1×10¹³ GC/kg. In one embodiment, the constructs may be delivered in volumes from 1 μL to about 100 mL. As used herein, the term “dosage” or “amount” can refer to the total dosage or amount delivered to the subject in the course of treatment, or the dosage or amount delivered in a single unit (or multiple unit or split dosage) administration.

The above-described recombinant vectors may be delivered to host cells according to published methods. The rAAV, preferably suspended in a physiologically compatible carrier, may be administered to a desired subject including a human. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

In another embodiment, the composition includes a carrier, diluent, excipient and/or adjuvant. In certain embodiments, for administration to a human patient, the rAAV is suitably suspended in an aqueous solution containing saline, a surfactant, and a pharmaceutically and/or physiologically compatible salt or mixture of salts. Suitably, the formulation is adjusted to a physiologically acceptable pH, e.g., in the range of pH 6 to 9, or pH 6.0 to 7.5, or pH 6.2 to 7.7, or pH 6.5 to 7.5, pH 7.0 to 7.7, or pH 7.2 to 7.8, or about 7.0. In certain embodiments, the formulation is adjusted to a pH of about 6.0, about 6.1, about 6.2, about 6.3, about 6.4, about 6.5, about 6.6, about 6.7, about 6.8, about 6.9, about 7.0, about 7.1, about 7.2, about 7.3 about 7.4, about 7.5, about 7.6, about 7.7, or about 7.8. In certain embodiments, a pH of about 7.28 to about 7.32, about 6.0 to about 7.5, about 6.2 to about 7.7, about 7.5 to about 7.8, about 6.0, about 6.1, about 6.2, about 6.3, about 6.4, about 6.5, about 6.6, about 6.7, about 6.8, about 6.9, about 7.0, about 7.1, about 7.2, about 7.3 about 7.4, about 7.5, about 7.6, about 7.7, or about 7.8 may be desired. In certain embodiments, for intravenous delivery, a pH of about 6.8 to about 7.2 may be desired. However, other pHs within the broadest ranges and these subranges may be selected for other route of delivery.

Optionally, the compositions of the invention may contain, in addition to the rAAV and/or variants and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

Dosages of the vector depends primarily on factors such as the condition being treated, the age, weight and health of the patient, and may thus vary among patients. For example, a therapeutically effective human dosage of viral vector is generally in the range of from about 25 to about 1000 microliters to about 100 mL of solution containing concentrations of from about 1×10⁹ to 1×10¹⁶ genomes virus vector (to treat an average subject of 70 kg in body weight) including all integers or fractional amounts within the range, and preferably 1.0×10¹² GC to 1.0×10¹³ GC for a human patient. The composition of the invention may be delivered in a volume of from about 0.1 μL to about 10 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. In another embodiment, the volume is about 1.5 mL. In another embodiment, the volume is about 2 mL. In another embodiment, the volume is about 2.5 mL. In another embodiment, the volume is about 3 mL. In another embodiment, the volume is about 3.5 mL. In another embodiment, the volume is about 4 mL. In another embodiment, the volume is about 5 mL. In another embodiment, the volume is about 5.5 mL. In another embodiment, the volume is about 6 mL. In another embodiment, the volume is about 6.5 mL. In another embodiment, the volume is about 7 mL. In another embodiment, the volume is about 8 mL. In another embodiment, the volume is about 8.5 mL. In another embodiment, the volume is about 9 mL. In another embodiment, the volume is about 9.5 mL. In another embodiment, the volume is about 10 mL.

In some embodiments, a concentration of a recombinant adeno-associated virus carrying a nucleic acid sequence encoding the desired transgene under the control of the regulatory sequences desirably ranges from about 10⁷ and 10¹⁴ genome copies per milliliter (GC/mL) in a composition.

In one embodiment, the dosage of rAAV in a composition is from about 1.0×10⁹ GC/kg of body weight to about 1.5×10¹³ GC/kg. In one embodiment, the dosage is about 1.0×10¹⁰ GC/kg. In one embodiment, the dosage is about 1.0×10¹¹ GC/kg. In one embodiment, the dosage is about 1.0×10¹² GC/kg. In one embodiment, the dosage is about 5.0×10¹² GC/kg. In one embodiment, the dosage is about 1.0×10¹³ GC/kg. All ranges described herein are inclusive of the endpoints.

In one embodiment, the effective dosage (total genome copies delivered) is from about 10⁷ to 10¹³ genome copies. In one embodiment, the total dosage is about 10⁸ genome copies. In one embodiment, the total dosage is about 10⁹ genome copies. In one embodiment, the total dosage is about 10¹⁰ genome copies. In one embodiment, the total dosage is about 10¹¹ genome copies. In one embodiment, the total dosage is about 10¹² genome copies. In one embodiment, the total dosage is about 10¹³ genome copies. In one embodiment, the total dosage is about 10¹⁴ genome copies. In one embodiment, the total dosage is about 10¹⁵ genome copies.

It is desirable that the lowest effective concentration of virus be utilized in order to reduce the risk of undesirable effects, such as toxicity. Still other dosages and administration volumes in these ranges may be selected by the attending physician, taking into account the physical state of the subject, preferably human, being treated, the age of the subject, the particular disorder and the degree to which the disorder, if progressive, has developed.

In certain embodiments, the composition comprises an rAAV comprising an inducible GLP-2 agonist construct. In certain embodiments, the inducing agent or molecule is a rapamycin or a rapalog. In certain embodiments, the inducing agent is rapamycin, and is administered at least one or more, at least two or more, at least three or more times following rAAV-comprising composition. In some embodiments the rapamycin is administered at dose at least about 4 to at least about 40 nM. In certain embodiments, the inducing agent (i.e., rapamycin) is administered at a dose at least about 0.1 mg/kg to at least about 3.0 mg/kg. In certain embodiments, the inducing agent (i.e., rapamycin) is administered at a dose at least about 0.5 mg/kg to at least about 2.0 mg/kg.

The viral vectors and other constructs described herein may be used in preparing a medicament for delivering a GLP-2 fusion protein construct to a subject in need thereof, supplying GLP-2 having an increased half-life to a subject, and/or for treating SBS in a subject. Thus, in another aspect a method of treating SBS is provided. The method includes administering a composition as described herein to a subject in need thereof. In one embodiment, the composition includes a viral vector containing a GLP-2 fusion protein expression cassette, as described herein.

As used herein, the term “treatment” or “treating” is defined encompassing administering to a subject one or more compounds or compositions described herein for the purposes of amelioration of one or more symptoms of short bowel syndrome. “Treatment” can thus include one or more of reducing progression of SBS, reducing the severity of the symptoms, removing the disease symptoms, delaying progression of disease, or increasing efficacy of therapy in a given subject.

In another embodiment, a method for treating SBS in a subject is provided. The method includes administering a viral vector comprising a nucleic acid molecule comprising a sequence encoding a fusion protein as described herein. In one embodiment, the subject is a human.

A course of treatment may optionally involve repeat administration of the same viral vector (e.g., an AAVrh91 vector) or a different viral vector (e.g., an AAVrh91 and an AAV3B.AR2.12). Still other combinations may be selected using the viral vectors described herein. Optionally, the composition described herein may be combined in a regimen involving nutritional therapy (enteral or parenteral nutrition), medications, such as those used to control stomach acid, reduce diarrhea, or improve intestinal absorption, or a GLP-2 analog, or surgery. In certain embodiments, the AAV vector and the combination therapy are administered essentially simultaneously. In other embodiments, the AAV vector is administered first. In other embodiments, the combination therapy is delivered first.

In one embodiment, the composition is administered in combination with an effective amount of a GLP-2 analog. Various commercially available GLP-2 products are known in the art, including, without limitation, teduglutide, glepaglutide, and apraglutide.

In some embodiments, combination of the rAAV described herein with GLP-2 analog decreases GLP-2 analog dose requirements in the subject, as compared to prior to treatment with the viral vector. Such dose requirements may be reduced by 10% or more, 20% or more, 30% or more, 40% or more, 50% or more, 60% or more, 70% or more, 80% or more, or 90% or more. The treating physician may determine the correct dosage of GLP-2 analog needed by the subject. For example, the subject may be being treated using GLP-2 analog or other therapy, which the treating physician may continue upon administration of the AAV vector. Such GLP-2 analog or other co-therapy may be continued, reduced, or discontinued as needed subsequently.

In one embodiment, composition comprising the expression cassette, vector genome, rAAV, or other composition described herein for gene therapy is delivered as a single dose per patient. In one embodiment, the subject is delivered a therapeutically effective amount of a composition described herein. As used herein, a “therapeutically effective amount” refers to the amount of the expression cassette or vector, or a combination thereof that delivers and expresses in the target cells an amount of GLP-2-Fc sufficient to reach therapeutic goal. The therapeutically effective amount may be selected by the treating physician, or guided based on previously determined guidelines. For example, teduglutide may be provided at an initial dose of 0.05 mg/kg subcutaneously daily. The dose may be increased in 0.025 mg/kg increments for subjects with moderate-to-severe renal impairment. The rAAV may be delivered to the subject and then supplemented with oral or subcutaneous teduglutide, or other medication as needed to reach the equivalent of the desired dosage of 0.05 mg/kg daily.

In certain embodiments, the therapeutic goal is to ameliorate or treat one or more of the symptoms of SBS. A therapeutically effective amount may be determined based on an animal model, rather than a human patient.

As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. SEQ ID NO: 32 provides the encoded amino acid sequence of the AAVrh91 vp1 protein. The term “heterogenous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine—glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine—glycine pairs.

As used herein, a “stock” of rAAV refers to a population of rAAV. Despite heterogeneity in their capsid proteins due to deamidation, rAAV in a stock are expected to 5 share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected. As used herein the terms “GLP-2 construct”, “GLP-2 expression construct” and synonyms include the GLP-2 sequence as described herein in combination with a leader and fusion domain. The terms “GLP-2 construct”, “GLP-2 expression construct” and synonyms can be used to refer to the nucleic acid sequences encoding the GLP-2 fusion protein or the expression products thereof.

The terms “percent (%) identity”, “sequence identity”, “percent sequence identity”, or “percent identical” in the context of nucleic acid sequences refers to the bases in the two sequences which are the same when aligned for correspondence. The length of sequence identity comparison may be over the full-length of the genome, the full-length of a gene coding sequence, or a fragment of at least about 100 to 150 nucleotides, or as desired. However, identity among smaller fragments, e.g., of at least about nine nucleotides, usually at least about 20 to 24 nucleotides, at least about 28 to 32 nucleotides, at least about 36 or more nucleotides, may also be desired. Multiple sequence alignment programs are also available for nucleic acid sequences. Examples of such programs include, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

By the term “highly conserved” is meant at least 80% identity, preferably at least 90% identity, and more preferably, over 97% identity. Identity is readily determined by one of skill in the art by resort to algorithms and computer programs known by those of skill in the art.

Unless otherwise specified by an upper range, it will be understood that a percentage of identity is a minimum level of identity and encompasses all higher levels of identity up to 100% identity to the reference sequence. Unless otherwise specified, it will be understood that a percentage of identity is a minimum level of identity and encompasses all higher levels of identity up to 100% identity to the reference sequence. For example, “95% identity” and “at least 95% identity” may be used interchangeably and include 95%, 96%, 97%, 98%, 99%, and up to 100% identity to the referenced sequence, and all fractions therebetween.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 70 amino acids to about 100 amino acids, or a peptide fragment thereof or the corresponding nucleic acid sequence coding sequencers. A suitable amino acid fragment may be at least about 8 amino acids in length, and may be up to about 150 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

“Patient” or “subject” as used herein means a mammalian animal, including a human, a veterinary or farm animal, a domestic animal or pet, and animals normally used for clinical research. In one embodiment, the subject of these methods and compositions is a human. In another embodiment, the subject is not a feline.

As used herein, the term “about” means a variability of 10% (±10%, e.g., ±1, ±2, ±3, ±4, ±5, ±6, ±7, ±8, ±9, ±10, or values therebetween) from the reference given, unless otherwise specified.

In certain instances, the term “E+#” or the term “e+#” is used to reference an exponent. For example, “5E10” or “5e10” is 5×10¹⁰. These terms may be used interchangeably.

The term “regulation” or variations thereof as used herein refers to the ability of a composition to inhibit one or more components of a biological pathway.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

A reference to “one embodiment” or “another embodiment” in describing an embodiment does not imply that the referenced embodiment is mutually exclusive with another embodiment (e.g., an embodiment described before the referenced embodiment), unless expressly specified otherwise.

### EXAMPLES

The following examples are provided to illustrate various embodiments of the present invention. The EXAMPLES are not intended to limit the present invention in any way.

### Example 1—Construction of GLP-2 Vectors

GLP-2 agonists are challenging to express via adeno-associated virus (AAV). GLP-2 is normally expressed from the glucagon precursor protein, which requires tissue specific proteases and produces unwanted proteins. Expression systems using traditional heterologous signal peptides yield low expression. Expression systems using heterologous propeptides with universal protease cleavage sites yield foreign protein sequences that could be targets for T cells.

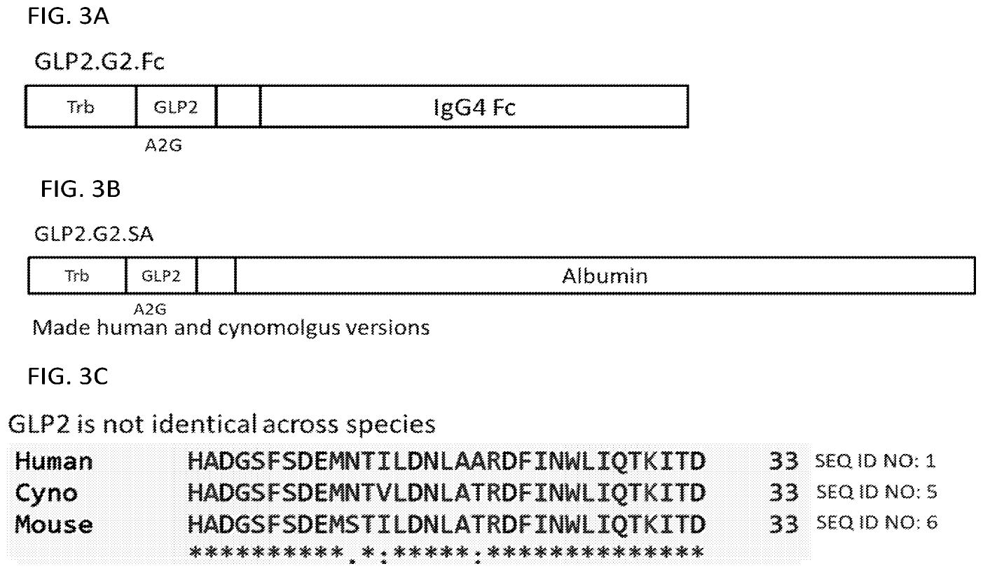

More specifically, vectors were constructed in which a leader sequence was placed upstream of one of several GLP-2 receptor agonist amino acid sequences followed by a fusion domain. See, e.g., FIG. 3. The resulting protein sequence was back-translated, followed by addition of a kozak consensus sequence, stop codon, and cloning sites. The sequences were produced, and cloned into an expression vector containing a CMV promoter under the control of an inducible expression system. The expression construct was flanked by AAV2 ITRs. The resulting plasmid is called pAAV.Z121.hGLP2.G2.Fc.rBG.

In a second construct, the sequences were produced, and cloned into an expression vector containing a CB7 constitutive promoter.

### Example 2—In Vitro Expression

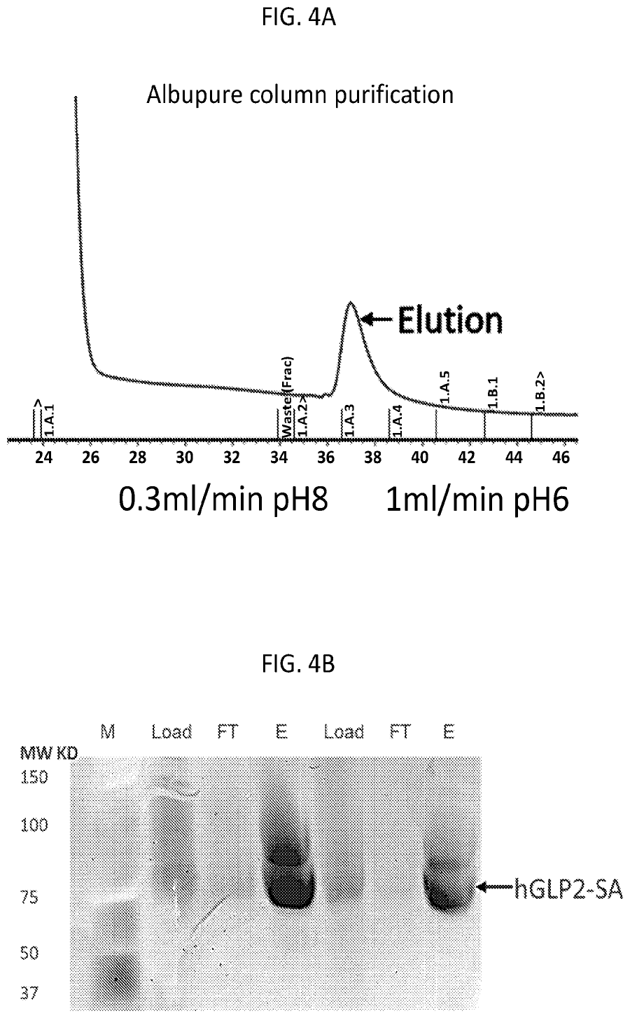

The following constructs were packaged in an AAVrh91 vector by triple transfection, as previously described.
AAVrh91.CB7.CI.hGLP-2-Fc.rBG
AAVrh91.CB7.CI.hGLP-2-SA.rBG
   GLP-2-SA fusions were measured in culture supernatants of HEK293 cells transfected with vector for inducible human GLP-2-SA with human Thrombin signal sequence. GLP2-SA was identified by gel electrophoresis using spyroRuby stain. FIG. 4A and FIG. 4B.

### Example 3—Pilot Expression in Rag1KO Mice

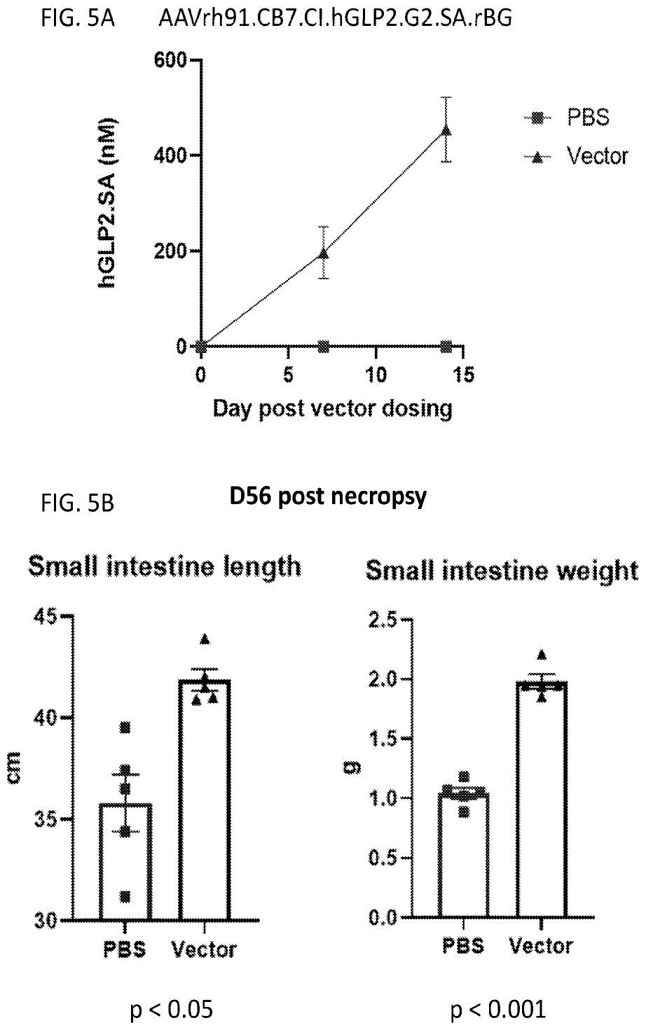

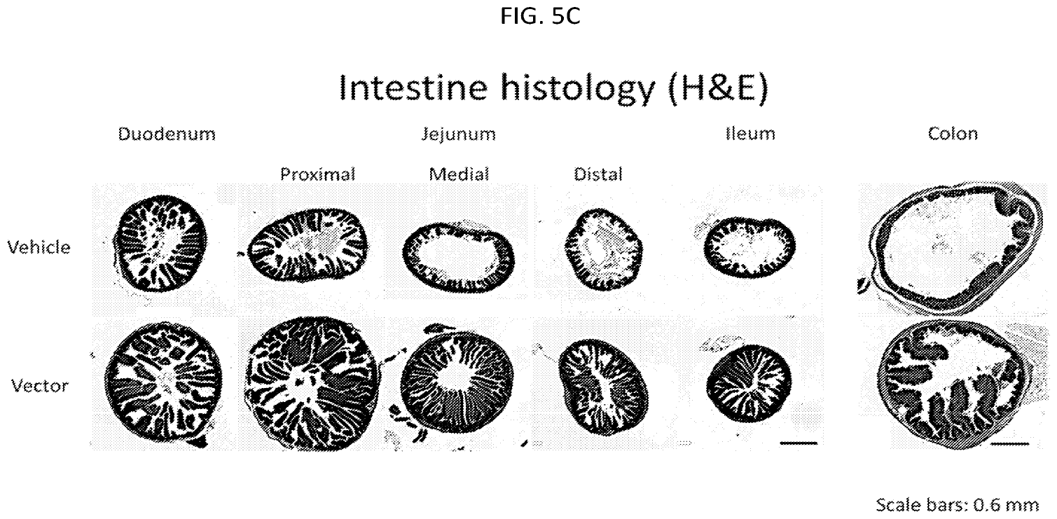

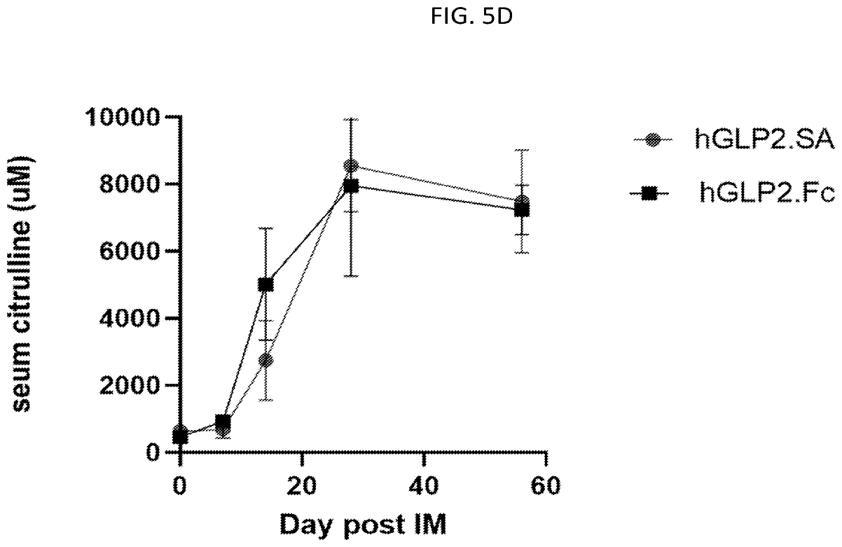

Rag1KO female mice were treated with an injection of the vector AAVrh91.CB7.CI.hGLP-2-SA.rBG (1×10¹¹ GC/mouse) or AAVrh91.CB7.CI.hGLP-2-Fc.rBG (1×10¹¹ GC/mouse) via IM route of administration. Serum was serially collected by separating whole blood in serum separator tubes containing 5 microliters DPP-IV inhibitor (Millipore) and assayed for active GLP-2 expression and activity as above. Vector was injected at day 0 and mice were necropsied at day 56. Serum GLP-2 concentrations (nM) are shown in FIG. 5A and are an estimation based on Fc fusion standard. Serum expression levels reached increased through day 14 post dosing.

Small intestines were weighed and measured at necropsy. The length and weight of small intestine increased significantly as compared to control animals (FIG. 5B). In addition, vector treated intestine show healthy enterocyte growth as compared to vehicle treated animals. (FIG. 5C). Serum citrulline levels (uM), a biomarker of gut surface area, were measured. (FIG. 5D).

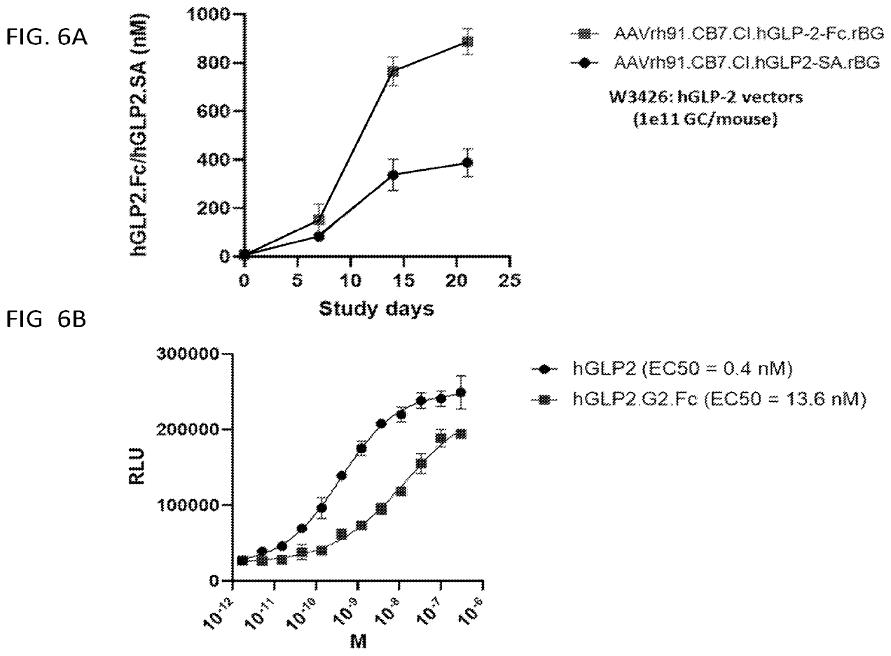

Expression of GLP-2 was compared with albumin fusion construct. Fc fusion construct showed greater GLP-2 expression (FIG. 6A). The GLP.G2.Fc fusion shows significantly lower potency (EC50=13.6 nM) as compared to hGLP (EC50=0.4 nM) (FIG. 6B). These data indicate that AAV-mediated expression of GLP-2 agonist demonstrates substantial and durable increases in gut surface area which should be therapeutic in short bowel syndromes.

### Example 4—Dosage Study in NHPs

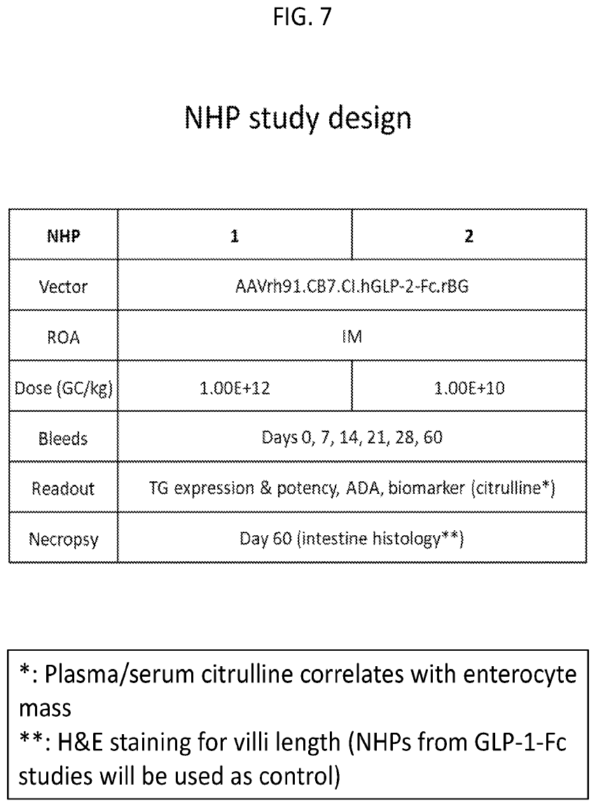

In this study, we examine expression of human GLP-2 in nonhuman primates (NHPs; i.e., rhesus macaques). FIG. 7 shows an outline of the study. Briefly, 2 NHPs were administered AAVrh91.CB7.CI.hGLP-2-Fc.rBG via intramuscular injection (IM) at a dose of 1×10¹³ (1e13) GC/kg (E185NG) and a of dose 5×10¹⁰ (1e10) GC/kg (BM239H).

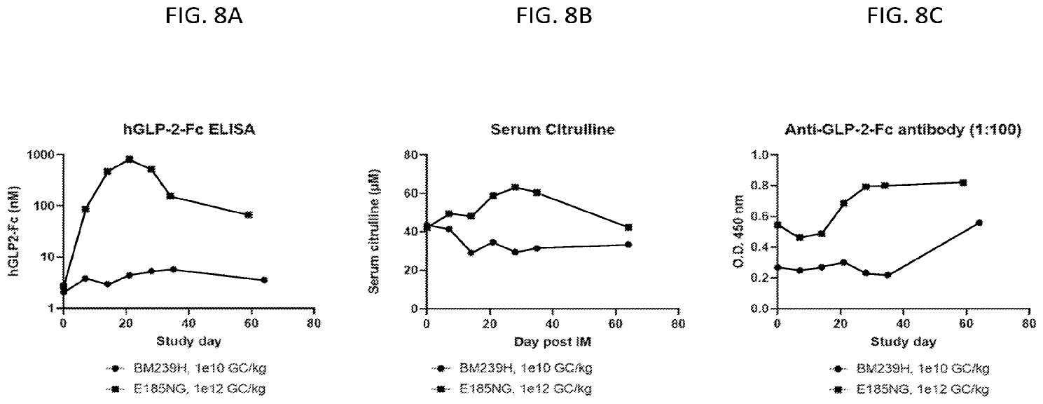

GLP-2 expression and potency, liver enzymes, and citrulline levels were measured. Necropsy is performed at day 60. FIG. 8A shows plasma level of GLP-2-Fc fusion protein. FIG. 8B shows serum citrulline, a biomarker of gut surface area. FIG. 8C shows detection of anti-GLP-2-Fc antibody in NHP serum at 1:100 dilution. AAV-mediated expression of GLP-2-Fc fusion demonstrates substantial increase in gut surface area at the high dose until anti-GLP-2-Fc antibodies reduced its expression demonstrating its therapeutic efficacy on short bowel syndromes.

### Example 5—Long Term Efficacy and Safety Study in Rag1 KO Mice

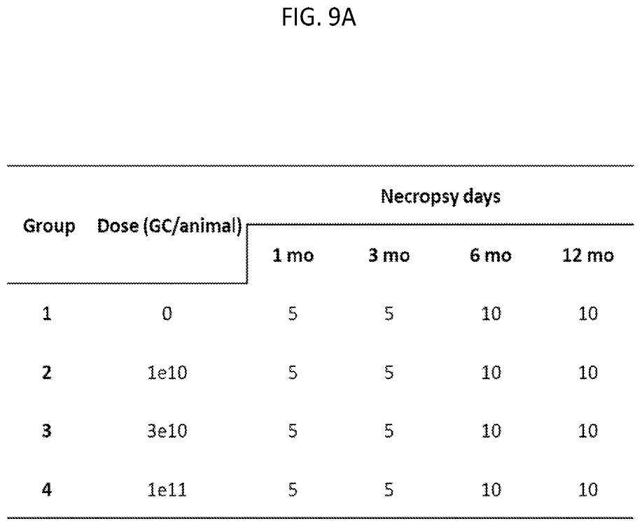

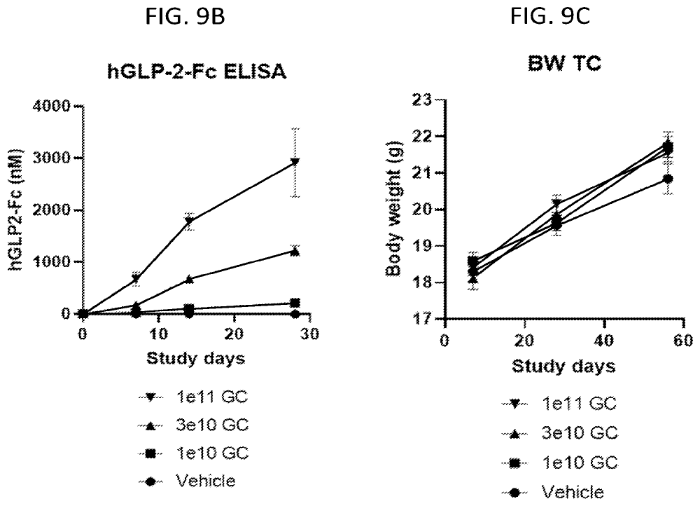

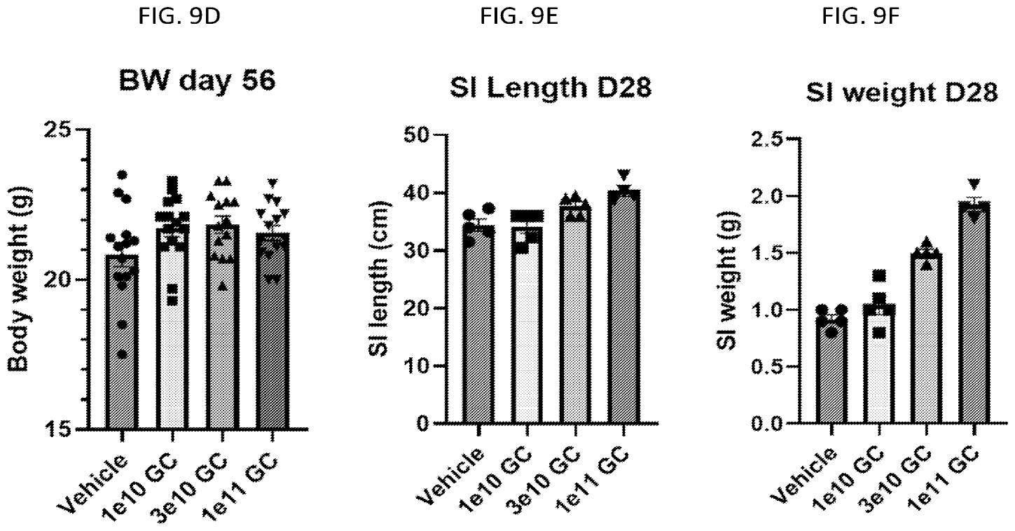

Rag1KO female mice were treated with an injection of the vector AAVrh91.CB7.CI.hGLP-2-Fc.rBG at a dosage of 1×10¹⁰ GC/mouse, 3×10¹⁰ GC/mouse, or 1×10¹¹ GC/mouse via IM route of administration. The study design is shown in FIG. 9A. hGLP2-Fc levels and body weight were measured throughout the study. The highest vector dosage showed highest serum GLP2 levels (FIG. 9B), while body weights were relatively consistent amongst all groups, with vehicle trending lowest (FIG. 9C, D). At day 28 necropsy, small intestine length and weight were measured. While SI length showed modest increase with dosage increase as compared to vehicle (FIG. 9E), SI weight was significantly increased in the two highest dosage groups (FIG. 9F).

All documents cited in this specification, are incorporated herein by reference. U.S. Provisional Patent Application No. 63/316,219, filed Mar. 3, 2022, is incorporated herein by reference. While the invention has been described with reference to particular embodiments, it will be appreciated that modifications can be made without departing from the spirit of the invention. Such modifications are intended to fall within the scope of the appended claims.

1. A composition comprising a nucleic acid comprising a sequence encoding a fusion protein comprising a GLP-2 protein and a IgG4 Fc, albumin or XTEN polypeptide, wherein the fusion protein has the sequence of SEQ ID NO: 13, 15, 17, 19, 21, or 23, or a sequence at least 99% identical thereto.

2. The composition according to claim 1, wherein the sequence encoding the fusion protein is SEQ ID NO: 14, 16, 18, 20, 22, or 24, or a sequence sharing at least 75% identical thereto.

3. The composition comprising a viral vector comprising:
(a) an adeno-associated virus (AAV) capsid, and
(b) a vector genome packaged in the AAV capsid, said vector genome comprising AAV inverted terminal repeats (ITRs), the coding sequence for comprising a GLP-2 protein and a IgG4 Fc, albumin or XTEN polypeptide, wherein the fusion protein has the sequence of SEQ ID NO: 13, 15, 17, 19, 21, or 23, or a sequence at least 99% identical thereto, and regulatory sequences which direct expression of the fusion protein.

4. The composition according to claim 1, wherein the viral vector is an rAAV having the AAV capsid of AAVrh91 or AAVhu68.

5. The composition according to claim 1, wherein the fusion protein is under the control of an inducible gene expression system that comprises a regulatable promoter, an activation domain, and a DNA binding domain.

6. (canceled)

7. The composition according to claim 3, wherein the AAV inverted terminal repeats (ITRs) are an AAV2 5′ ITR and an AAV2 3′ ITR which flank the fusion protein coding sequence and regulatory sequences.

8. The composition according to claim 3, wherein the vector genome comprises a CB7 promoter and a rabbit globin poly A.

9. The composition according to claim 5, wherein the inducible gene expression system comprises
(a) an activation domain comprising a transactivation domain and a FKBP12-rapamycin binding (FRB) domain of FKBP12-rapamycin-associated protein (FRAP);
(b) a DNA binding domain comprising a zinc finger homeodomain (ZFHD) and one, two or three FK506 binding protein domain (FKBP) subunit genes; and
(c) at least one copy of the binding site for ZFHD followed by a minimal promoter, and
(d) a regulatable promoter.

10. The composition according to claim 9, wherein the inducible gene expression system is comprised in one vector.

11. The composition according to claim 9, wherein the inducible gene expression system is comprised in two vectors.

12. The composition according to claim 9, wherein the transactivation domain comprises a portion of NF-κB p65.

13. The composition according to claim 9, wherein the regulatable promoter is a constitutive promoter.

14. The composition according to claim 12, wherein the regulatable promoter is a CMV promoter.

15. The composition according to claim 9, further comprising an IRES or 2A.

16. The composition according to claim 9, comprising at least 8 copies of the binding site for ZFHD.

17. A composition according to claim 5 comprising a regulatable promoter; an activation domain comprising a p65 transactivation domain and a FKBP12-rapamycin binding (FRB) domain of FKBP12-rapamycin-associated protein (FRAP); a DNA binding domain comprising a zinc finger homeodomain (ZFHD) and three FK506 binding protein domain (FKBP) subunit genes; 12 copies of the binding site for ZFHD, and a sequence encoding a fusion protein comprising a GLP-2 analog and a human IgG4 Fc.

18-20. (canceled)

21. The composition according to any one of claims 1 to 20, wherein the composition is formulated to be administered a dose of 1×10⁹ GC/kg to 5×10¹³ GC/kg of the rAAV or 1×10¹⁰ to 1.5×10¹⁵ GC of the rAAV.

23. The composition according to any one of claims 1 to 22, wherein the rAAV is delivered intramuscularly or intravenously.

24. A method of treating a subject having a metabolic disease, comprising delivering to the subject a recombinant adeno-associated virus (rAAV) having an AAV capsid from adeno-associated virus rh91 or hu68, and a vector genome packaged in the AAV capsid, said vector genome comprising AAV inverted terminal repeats (ITRs), a sequence encoding a fusion protein comprising a GLP-2 analog and a human IgG4 Fc, and regulatory sequences which direct expression of the fusion protein.

25-28. (canceled)


SEQUENCE LISTING (55 sequences)

SEQ ID NO: 1 - protein, 33 aa, Homo sapiens
HADGSFSDEMNTILDNLAARDFINWLIQTKITD

SEQ ID NO: 2 - protein, 33 aa, Homo sapiens
HGDGSFSDEMNTILDNLAARDFINWLIQTKITD

SEQ ID NO: 3 - protein, 39 aa, synthetic construct
  REGION 1..39: note = synthetic construct
HGEGTFSSELATILDALAARDFIAWLIATKITDKKKKKK

SEQ ID NO: 4 - protein, 30 aa, Homo sapiens
HAEGTFTSDVSSYLEGQAAKEFIAWLVKGR

SEQ ID NO: 5 - protein, 33 aa, Macaca fascicularis
HADGSFSDEMNTVLDNLATRDFINWLIQTKITD

SEQ ID NO: 6 - protein, 33 aa, Mus musculus
HADGSFSDEMSTILDNLATRDFINWLIQTKITD

SEQ ID NO: 7 - protein, 43 aa, Homo sapiens
MAHVRGLQLPGCLALAALCSLVHSQHVFLAPQQARSLLQRVRR

SEQ ID NO: 8 - protein, 229 aa, Homo sapiens
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - protein, 224 aa, Macaca mulatta
PPCPPCPAPELLGGPSVFLFPPKPKDTLMISRTPEVTCVVVDVSQEDPEVQFNWYVDGVEVHNAQTKPRERQFNSTYRVVSVLTVTHQDWLNGKEYTCKVSNKGLPAPIEKTISKAKGQPREPQVYILPPPQEELTKNQVSLTCLVTGFYPSDIAVEWESNGQPENTYKTTPPVLDSDGSYLLYSKLTVNKSRWQPGNIFTCSVMHEALHNHYTQKSLSVSPGK

SEQ ID NO: 10 - protein, 584 aa, Homo sapiens
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALG

SEQ ID NO: 11 - protein, 584 aa, Macaca mulatta
DTHKSEVAHRFKDLGEEHFKGLVLVAFSQYLQQCPFEEHVKLVNEVTEFAKTCVADESAENCDKSLHTLFGDKLCTVATLRETYGEMADCCAKQEPERNECFLQHKDDNPNLPPLVRPEVDVMCTAFHDNEATFLKKYLYEVARRHPYFYAPELLFFAARYKAAFAECCQAADKAACLLPKLDELRDEGKASSAKQRLKCASLQKFGDRAFKAWAVARLSQKFPKAEFAEVSKLVTDLTKVHTECCHGDLLECADDRADLAKYMCENQDSISSKLKECCDKPLLEKSHCLAEVENDEMPADLPSLAADYVESKDVCKNYAEAKDVFLGMFLYEYARRHPDYSVMLLLRLAKAYEATLEKCCAAADPHECYAKVFDEFQPLVEEPQNLVKQNCELFEQLGEYKFQNALLVRYTKKVPQVSTPTLVEVSRNLGKVGAKCCKLPEAKRMPCAEDYLSVVLNRLCVLHEKTPVSEKVTKCCTESLVNRRPCFSALELDEAYVPKAFNAETFTFHADMCTLSEKEKQVKKQTALVELVKHKPKATKEQLKGVMDNFAAFVEKCCKADDKEACFAEEGPKFVAASQAALA

SEQ ID NO: 12 - protein, 15 aa, synthetic construct
  REGION 1..15: note = synthetic construct
GGGGSGGGGSGGGGS

SEQ ID NO: 13 - protein, 322 aa, synthetic construct
  REGION 1..322: note = synthetic construct
MAHVRGLQLPGCLALAALCSLVHSQHVFLAPQQARSLLQRVRRHGDGSFSDEMNTILDNLAARDFINWLIQTKITDGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - DNA, 969 bp, synthetic construct
  misc_feature 1..969: note = synthetic construct
  source 1..969: mol_type = other DNA
atggctcacgttcggggactgcagctgcctggatgtctggctcttgccgctctgtgtagcctggtgcacagccagcacgtgtttctggctcctcagcaagccagatcactgctgcagagagttagaaggcacggcgacggcagcttcagcgacgagatgaacaccatcctggacaacctggccgccagagacttcatcaactggctgatccagaccaagatcaccgacggtggcggaggcggaggatctggtggtggtggatctggcggcggaggttctgccgagtctaagtacggacctccttgtcctccctgtcctgctccagaagctgctggcggcccatccgtgtttctgttccctccaaagcctaaggacaccctgatgatcagcagaacccctgaagtgacctgcgtggtggtcgacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggactccgatggctcattcttcctgtacagcagactgaccgtggacaagagcaggtggcaagagggcaacgtgttcagctgcagcgtgatgcacgaggccctgcacaaccactacacccagaaaagcctgagcctgtctctgggctaa

SEQ ID NO: 15 - protein, 677 aa, synthetic construct
  REGION 1..677: note = synthetic construct
MAHVRGLQLPGCLALAALCSLVHSQHVFLAPQQARSLLQRVRRHGDGSFSDEMNTILDNLAARDFINWLIQTKITDGGGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALG

SEQ ID NO: 16 - DNA, 2034 bp, synthetic construct
  misc_feature 1..2034: note = synthetic construct
  source 1..2034: mol_type = other DNA
atggctcacgttcggggactgcagctgcctggatgtctggctcttgccgctctgtgtagcctggtgcacagccagcacgtgtttctggctcctcagcaagccagatcactgctgcagagagttagaaggcacggcgacggcagcttcagcgacgagatgaacaccatcctggacaacctggccgccagagacttcatcaactggctgatccagaccaagatcaccgacggtggcggaggcggaggatctggtggtggtggatctggcggcggaggttctgacgcccacaaatctgaagtggcccaccggttcaaggacctgggcgaagagaatttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggctgcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaacctgggcaaagtgggcagcaagtgttgcaagcaccctgaggccaagagaatgccctgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaagtgctgcaaggccgacgacaaagagacctgcttcgccgaagagggcaagaaactggtggccgcttctcaggctgctctgggataa

SEQ ID NO: 17 - protein, 957 aa, synthetic construct
  REGION 1..957: note = synthetic construct
MAHVRGLQLPGCLALAALCSLVHSQHVFLAPQQARSLLQRVRRHGDGSFSDEMNTILDNLAARDFINWLIQTKITDGGGGGGSGGGGSGGGGSSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPG

SEQ ID NO: 18 - DNA, 2874 bp, synthetic construct
  misc_feature 1..2874: note = synthetic construct
  source 1..2874: mol_type = other DNA
atggctcacgttcggggactgcagctgcctggatgtctggctcttgccgctctgtgtagcctggtgcacagccagcacgtgtttctggctcctcagcaagccagatcactgctgcagagagttagaaggcacggcgacggcagcttcagcgacgagatgaacaccatcctggacaacctggccgccagagacttcatcaactggctgatccagaccaagatcaccgacggtggcggaggcggaggatctggtggtggtggatctggcggcggaggaagttctcctgctggcagccctacaagcaccgaggaaggcacaagcgagtctgccacacctgagtctggccctggcacatctacagagcctagcgaaggatctgccccaggatctcctgccggctctccaacatctaccgaagagggaaccagcaccgagccatctgagggatctgctcccggaacaagcacagagccttcagaaggatccgctcctggcacctctgaaagcgccacaccagaaagcggacctggatctgagcctgccacaagcggatctgagacacctggaagcgagccagccacatctggcagcgaaacacctggttctccagccggatctcccaccagcacagaagagggcacatccgaatctgctacccctgaatctggaccaggcacctccacagaaccttccgagggttctgcccctggaacctctaccgaaccatcagaaggcagcgctccaggttcaccagctggaagcccaacctctacagaggaagggacatccactgagccaagcgagggaagcgctcccggcactagtacagaaccgagcgagggcagtgctcctggaaccagcgaatccgctactccagagagtggcccaggcaccagtactgaaccctctgagggtagcgcacccggaacatctgagagcgctactcccgaatcaggcccaggctctgaacctgctaccagcggaagtgaaacacccggcacctctactgagccctccgaaggctcagcacctggcacaagcactgaaccatcagagggctccgcaccaggcaccagcgaaagtgctacaccagagtcaggacccggaacctccgaaagtgcaactcctgagagcggaccaggctctcccgctggatctcctacatcaactgaagaagggacctccgagagcgcaaccccagagtctggtccaggatcagaacctgccacctccggctctgaaaccccaggcacttctgagtccgccacgccagaatctggtcctgggactagcaccgaaccgagtgaaggttcagctcccgggacttctacggaacccagtgaaggatctgcacccggcacatcaaccgaaccgtcagagggatcagcccctgggacttccacagagccgtctgagggcagcgccccagggacgtctacagaaccatctgaaggatcagcaccagggacctctaccgagccaagtgaaggcagtgcaccgggaagtccagcaggctcccctacaagtactgaagagggtactagcacggaacccagcgagggttccgctccagggacatctgaatccgcaactccggaatccggacctggcagtgaaccagctacatccggatccgagactccgggaacctcagaatcagctacacccgagagtggacctggctccgaaccagcaactagcggctcagaaactcctgggacaagcgagagtgcaacacccgaatctggacctggaacaagtactgagcccagcgaaggcagcgcccctggaacttctgaatctgccactcctgaaagtggccctggaagccctgcaggctcacccacatccacagaagaaggatcaccagcaggcagccccacttcaacggaagagggatccccagctggatccccaactagtacggaagaaggcacttcagaaagcgctacgcccgagtccggtcctggcacttctactgaaccatccgagggaagtgcccctggcacttccgagagtgctacacctgaaagcggtcccggctctgaaccagccacttctggatctgaaacgcccgggacatccgagtcagcaacgcccgaaagcggcccaggttccgagccggctactagtggttcagagactccagggacttccgagtctgctactcctgagtccggaccgggaacatcaaccgagccttccgaaggatctgcacctggaagccctgccggatctcctaccagtactgaggaaggcacctcagagtctgccactccagagtcaggtcctggaagcgaacctgcaacaagcggcagcgaaactccaggcactagcgagtcagctaccccagaatcaggacctggatctccagcagggtccccaacatctactgaggaaggctctcctgctggctcccctacctctaccgaagaggggacctcaacagagccatccgaggggagcgcacctggtacatcagagtccgcaactcccgagtctggccccggaactagcgaatctgcaaccccggaaagtggacccgggacgagtgaatcagccacacctgaatccggtccaggatccgagcctgcaacttctggaagcgagacaccaggatctgagccagctacgtctggctctgagactcctggatctcctgctggtagtcccacctccactgaagagggaacttccaccgaaccgagcgagggatcagcaccaggcactagcacagaaccgtccgaaggatctgctccaggctctgaacccgcaacctccggatcagaaacccctggaacatccgaaagcgctacaccggaaagtggccccggaacctctacagaacctagcgagggaagcgcaccaggataa

SEQ ID NO: 19 - protein, 322 aa, synthetic construct
  REGION 1..322: note = synthetic construct
MAHVRGLQLPGCLALAALCSLVHSQHVFLAPQQALSLLQRVRRHGDGSFSDEMNTVLVDNLATRDFINWLIQTKITDGGGGGGGSGGGGSGGGGSAEFTPPCPPCPAPELLGGPSVFLFPPKPKDTLMISRTPEVTCVVVDVSQEDPEVQFNWYVDGVEVHNAQTKPRERQFNSTYRVVSVLTVTHQDWLNGKEYTCKVSNKGLPAPIEKTISKAKGQPREPQVYILPPPQEELTKNQVSLTCLVTGFYPSDIAVEWESNGQPENTYKTTPPVLDSDGSYLLYSKLTVNKSRWQPGNIFTCSVMHEALHNHYTQKSLSVSPG

SEQ ID NO: 20 - DNA, 969 bp, synthetic construct
  misc_feature 1..969: note = synthetic construct
  source 1..969: mol_type = other DNA
atggctcacgttcggggactgcagctgcctggatgtctggctcttgccgctctgtgtagcctggtgcacagccagcatgtgtttctggctcctcagcaggccctgagcctgctgcaaagagttagaaggcacggcgacggcagcttcagcgacgagatgaataccgtgctggtggacaacctggccaccagagacttcatcaactggctgatccagaccaagatcaccgacggtggtggcggaggcggaggatctggtggcggtggttctggcggtggcggatctgctgagtttacccctccttgtcctccctgtcctgctccagaactgctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgcccagacaaagcccagagagcggcagttcaacagcacctacagagtggtgtccgtgctgaccgtgacacaccaggattggctgaacggcaaagagtacacctgtaaagtctccaacaagggcctgcctgctcctatcgagaaaaccatcagcaaggccaagggccagcctagagaaccccaggtgtacatcctgcctccacctcaagaggaactgaccaagaaccaggtgtccctgacctgtctggtcaccggcttctacccttccgatatcgccgtggagtgggagagcaacggacagcccgagaacacctacaagaccacacctccagtgctggacagcgacggctcttacctgctgtactccaagctgacagtgaacaagagccggtggcagcccggcaacatcttcacctgttctgtgatgcacgaggccctgcacaaccactacacccagaaaagcctgagcgtgtcccctggataa

SEQ ID NO: 21 - protein, 678 aa, synthetic construct
  REGION 1..678: note = synthetic construct
MAHVRGLQLPGCLALAALCSLVHSQHVFLAPQQALSLLQRVRRHGDGSFSDEMNTVLVDNLATRDFINWLIQTKITGGGGGGGSGGGGSGGGGSDTHKSEVAHRFKDLGEEHFKGLVLVAFSQYLQQCPFEEHVKLVNEVTEFAKTCVADESAENCDKSLHTLFGDKLCTVATLRETYGEMADCCAKQEPERNECFLQHKDDNPNLPPLVRPEVDVMCTAFHDNEATFLKKYLYEVARRHPYFYAPELLFFAARYKAAFAECCQAADKAACLLPKLDELRDEGKASSAKQRLKCASLQKFGDRAFKAWAVARLSQKFPKAEFAEVSKLVTDLTKVHTECCHGDLLECADDRADLAKYMCENQDSISSKLKECCDKPLLEKSHCLAEVENDEMPADLPSLAADYVESKDVCKNYAEAKDVFLGMFLYEYARRHPDYSVMLLLRLAKAYEATLEKCCAAADPHECYAKVFDEFQPLVEEPQNLVKQNCELFEQLGEYKFQNALLVRYTKKVPQVSTPTLVEVSRNLGKVGAKCCKLPEAKRMPCAEDYLSVVLNRLCVLHEKTPVSEKVTKCCTESLVNRRPCFSALELDEAYVPKAFNAETFTFHADMCTLSEKEKQVKKQTALVELVKHKPKATKEQLKGVMDNFAAFVEKCCKADDKEACFAEEGPKFVAASQAALA

SEQ ID NO: 22 - DNA, 2037 bp, synthetic construct
  misc_feature 1..2037: note = synthetic construct
  source 1..2037: mol_type = other DNA
atggctcacgttcggggactgcagctgcctggatgtctggctcttgccgctctgtgtagcctggtgcacagccagcatgtgtttctggctcctcagcaagccctgagcctgctgcaaagagttagaaggcacggcgacggcagcttcagcgacgagatgaataccgtgctggtggacaacctggccaccagagacttcatcaactggctgatccagaccaagatcaccggtggtggcggaggcggaggatctggtggcggtggttctggcggtggcggatctgatacacacaagtctgaggtggcccaccggttcaaggacctgggcgaagaacacttcaaaggcctggtgctggtcgccttcagccagtacctgcagcagtgccctttcgaggaacacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctccactcgtcagacccgaagtggacgtgatgtgcaccgccttccacgacaatgaggccaccttcctgaagaaatacctgtacgaggtggccagacggcacccctacttttacgcccctgaactgctgttctttgccgccaggtacaaggccgccttcgccgaatgttgtcaggccgctgataaggccgcttgcctgctgcctaagctggacgagcttagagacgagggcaaagccagctccgccaagcagagactgaagtgtgccagcctgcagaagttcggcgatagagcctttaaggcctgggccgtcgctagactgagccagaagtttcccaaggccgagtttgccgaggtgtccaagctcgtgaccgacctgacaaaggtgcacaccgagtgctgtcacggcgacctgctggaatgcgccgacgatagagccgacctggccaagtacatgtgcgagaaccaggacagcatcagcagcaagctgaaagagtgctgcgacaagcctctgctggaaaagagccactgtctggccgaggtggaaaacgacgagatgcccgccgatctgccttctctggccgccgattacgtggaaagcaaggacgtgtgcaagaactacgccgaggccaaggacgtgttcctgggcatgtttctgtacgagtacgcccgcagacaccccgactactctgttatgctgctgctgagactggccaaggcctacgaggccactctggaaaagtgttgtgccgccgctgatccccacgagtgttacgccaaagtgttcgacgagttccagccactggtggaagaaccccagaacctggtcaagcagaactgcgagctgttcgagcagctgggcgagtacaagttccagaacgccctgctcgtgcggtacaccaagaaggtgccccaggtttccacacctacactggttgaggtgtcccggaacctgggaaaagtgggcgccaagtgttgcaagctgcctgaggccaagagaatgccctgcgccgaggattacctgagcgtggtgctgaacagactgtgcgtgctgcacgagaaaacccctgtgtccgagaaagtgaccaagtgctgtaccgagagcctggtcaatcggaggccttgctttagcgccctggaactggacgaggcctacgtgcccaaggccttcaacgccgagacattcaccttccacgccgacatgtgtaccctgagcgagaaagaaaagcaagtgaagaaacagacagccctggtcgagctggttaagcacaagcctaaggccaccaaagaacaactgaagggcgtgatggacaacttcgccgcctttgtggaaaaatgctgcaaggccgacgacaaagaggcctgcttcgcagaagagggccctaagtttgtggccgcctctcaagctgctctggcttaa

SEQ ID NO: 23 - protein, 958 aa, synthetic construct
  REGION 1..958: note = synthetic construct
MAHVRGLQLPGCLALAALCSLVHSQHVFLAPQQALSLLQRVRRHGDGSFSDEMNTVLVDNLATRDFINWLIQTKITDGGGGGGSGGGGSGGGGSSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPG

SEQ ID NO: 24 - DNA, 2877 bp, synthetic construct
  misc_feature 1..2877: note = synthetic construct
  source 1..2877: mol_type = other DNA
atggctcacgttcggggactgcagctgcctggatgtctggctcttgccgctctgtgtagcctggtgcacagccagcatgtgtttctggctcctcagcaagccctgagcctgctgcaaagagttagaaggcacggcgacggcagcttcagcgacgagatgaataccgtgctggtggacaacctggccaccagagacttcatcaactggctgatccagaccaagatcaccgacggtggcggaggcggaggatctggtggtggtggatctggcggcggaggaagttctcctgctggcagccctacaagcaccgaggaaggcacaagcgagtctgccacacctgagtctggccctggcacatctacagagcctagcgaaggatctgccccaggatctcctgccggctctccaacatctaccgaagagggaaccagcaccgagccatctgagggatctgctcccggaacaagcacagagccttcagaaggatccgctcctggcacctctgaaagcgccacaccagaaagcggacctggctctgaacctgccacaagcggatctgagacacctggaagcgagccagccacatctggcagcgaaacacctggatcaccagccggatctcccacctctaccgaggaagggacatccgagagcgctaccccagaatctggaccaggcaccagcacagaaccctctgaaggttcagcccctggaacctctaccgaaccatcagaaggcagcgctccaggttctcccgctggatcccctacatccacagaagagggcacctccactgaacctagcgagggaagtgctcccggcacttccacagaaccatccgagggcagtgcacctggaaccagcgaatctgctacccctgagagtggacccggaacatccactgagccctccgagggttcagctccaggcacatcagaatccgccactccagagtccggaccaggatctgagccagctaccagcggctctgaaacacccggcactagtaccgagccaagcgagggtagcgcaccagggacaagtaccgaaccgtctgagggctccgcaccaggcacttccgaaagtgctactcctgaaagcggcccaggcactagcgaatccgcaacacccgagagcggtcctggaagtcctgcaggttcacctaccagcactgaagaggggactagcgagagcgcaactcctgaatcaggccctggatccgaacctgctacctccggaagtgaaacccctgggacaagcgaaagtgcaacgcccgagtcaggacccgggactagcacggaacccagtgaaggatctgcacccgggacatctaccgagccgtcagaaggttctgctccagggactagtactgagccttccgaaggttctgcacctggaacttccacagagcccagtgaaggcagtgcccctggcacaagcactgaaccgtccgaaggcagtgctcccgggaccagtacagaaccgagcgagggctctgctcctggtagtccagcaggatctccaactagcaccgaagaagggacttccaccgagccttccgagggaagcgctcctggaacatccgagtccgctacgccagagagtggcccaggttctgaacccgctacttccggctcagagactcctgggacttctgagtctgcaaccccggaaagtggtcctggtagcgaaccagcaactagcggaagcgagacacccggaacctcagagagtgctacaccggaatccggtccagggacgtctacggaaccgtctgaaggatcagctcccggcactagcgaaagcgctacacctgaaagtggtcccggatctccagcaggcagcccaacctctactgaagaaggttccccagctggaagccccacttccactgaggaaggctctcccgcaggctcacccactagtacggaagaaggcacgtccgagtctgctactcccgaatccggacctggaactagcactgagccaagcgaaggatcagcacccggaacctctgagtccgccacaccagaatctggtcctggttccgagcctgccacttcaggatcagaaaccccgggcacgagtgaatcagcaacgccggaatctggccccggaagcgaaccggctacgtctggatctgaaacgccagggacctccgaatcagctacgcctgagtctggtccagggacatccaccgaacctagtgaaggctccgcacctggaagccctgctggaagcccaacgagtactgaagagggcacttctgagagcgctacgcctgagtcaggacctggaagcgaacctgcaacatccggctcagaaacaccagggaccagcgaaagcgcaaccccagagagtggacctggatctccagctggctctcctactagtacagaggaaggcagccctgctggctccccaacgtcaacagaagaaggtactagcacagagcccagcgagggttccgctccgggaacttctgaatctgctacacccgagtcaggtcctggtacaagcgagtcagctacgcccgaaagtggacctggcacctcagagtctgcaactcctgagagcggtccaggatcagaaccagccacctctggctctgagacaccaggttctgagcctgcaacgtccggaagcgaaacaccaggcagtcctgccggaagtcctacttcaaccgaagaggggacctctacagagccatcagagggctctgcaccgggcacctcaacagaaccatctgaaggatccgcaccgggctctgagcctgctactagtggaagcgaaactcctggcaccagtgaatccgctactcccgagtctggcccgggaacgtctactgaaccatctgagggaagtgccccaggctaa

SEQ ID NO: 25 - DNA, 665 bp, synthetic construct
  misc_feature 1..665: note = synthetic construct
  source 1..665: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 26 - DNA, 529 bp, synthetic construct
  misc_feature 1..529: note = synthetic construct
  source 1..529: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatc

SEQ ID NO: 27 - DNA, 978 bp, synthetic construct
  misc_feature 1..978: note = synthetic construct
  source 1..978: mol_type = other DNA
ggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccacacgccacccttgtgtttgacgtggaactgcttaagctagagactagaggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatccccccccacgccaccctggtgttcgacgtggagctgctgaagctggagtga

SEQ ID NO: 28 - protein, 107 aa, synthetic construct
  REGION 1..107: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 29 - DNA, 18 bp, synthetic construct
  misc_feature 1..18: note = synthetic construct
  source 1..18: mol_type = other DNA
aatgatgggcgctcgagt

SEQ ID NO: 30 - DNA, 121 bp, synthetic construct
  misc_feature 1..121: note = synthetic construct
  source 1..121: mol_type = other DNA
aacattttgacacccccataatatttttccagaattaacagtataaattgcatctcttgttcaagagttccctatcactctctttaatcactactcacagtaacctcaactcctgccacaa

SEQ ID NO: 31 - DNA, 2211 bp, Adeno-Associated Virus rh91
  source 1..2211: mol_type = unassigned DNA
atggctgctgacggttatcttccagattggctcgaggacaacctttctgaaggcattcgtgagtggtgggctctgaaacctggagcccctaaacccaaagcgaaccaacaaaagcaggacgacggccggggtcttgtgcttccgggttacaaatacctcggacccttcaacggactcgacaaaggagagccggtcaacgcggcggacgcggcagccctcgaacacgacaaagcttacgaccagcagctcaaggccggtgacaacccgtacctccggtacaaccacgccgacgccgagtttcaggagcgtcttcaagaagatacgtcttttgggggcaaccttggcagagcagtcttccaggccaaaaagagggttcttgagccttttggtctggttgaggaagcagctaaaacggctcctggaaagaagaggcctgtagagcagtctcctcaggaaccggactcatcatctggtattggcaaatcgggccagcagcctgccaaaaaaagactaaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtatggatacctcaccctgaacaacggaagtcaagcggtgggacgctcatccttttactgcctggagtacttcccttcgcagatgctaaggactggaaataacttcaccttcagctataccttcgaggatgtaccttttcacagcagctacgctcacagccagagtttggatcgcttgatgaatcctcttattgatcagtatctgtactacctgaacagaacgcaaaatcaatctggaagtgcacaaaacaaggacctgctttttagccgggggtctcctgctggcatgtctgttcagcccaaaaattggctacctgggccctgctaccggcaacagagagtttcaaagactaaaacagacaacaacaacagtaactttacctggacaggtgccagcaaatataatctcaatggccgcgaatcgatcattaatccaggaaccgctatggccagtcacaaggacgatgaagacaaatttttccctatgagcggcgttatgatatttggcaaagaaaatgcaggagcaagtaacactgcattagataatgtaatgattacggatgaagaagagattaaagctaccaatcctgtggcaacagagagatttggaactgtggcagtcaacttgcagagctcaaatacagaccccgcaactggagacgtccatgtcatgggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcctcatcaaaaatactccggtaccggcaaatcctccggcagagttcagcgctacaaagtttgcttcatttatcactcagtactccactggacaggtcagcgtggaaattgagtgggagctacagaaagaaaacagcaaacgttggaatccagaggtgcagtacacttccaactacgcgaagtctgccaatgtggactttactgtagacaacaatggtctttatactgaacctcgccctattggaacccggtatctcacacgacccttgtaa

SEQ ID NO: 32 - protein, 736 aa, adeno-associated virus rh91
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKSGQQPAKKRLNFGQTGDSESVPDPQPLGEPPETPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 33 - DNA, 3326 bp, synthetic construct
  misc_feature 1..3326: note = synthetic construct
  source 1..3326: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcggggactgcagctgcctggatgtctggctcttgccgctctgtgtagcctggtgcacagccagcacgtgtttctggctcctcagcaagccagatcactgctgcagagagttagaaggcacggcgacggcagcttcagcgacgagatgaacaccatcctggacaacctggccgccagagacttcatcaactggctgatccagaccaagatcaccgacggtggcggaggcggaggatctggtggtggtggatctggcggcggaggttctgccgagtctaagtacggacctccttgtcctccctgtcctgctccagaagctgctggcggcccatccgtgtttctgttccctccaaagcctaaggacaccctgatgatcagcagaacccctgaagtgacctgcgtggtggtcgacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggactccgatggctcattcttcctgtacagcagactgaccgtggacaagagcaggtggcaagagggcaacgtgttcagctgcagcgtgatgcacgaggccctgcacaaccactacacccagaaaagcctgagcctgtctctgggctaataaggtacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 34 - DNA, 4379 bp, synthetic construct
  misc_feature 1..4379: note = synthetic construct
  source 1..4379: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccagaagccaccatggctcacgttcggggactgcagctgcctggatgtctggctcttgccgctctgtgtagcctggtgcacagccagcacgtgtttctggctcctcagcaagccagatcactgctgcagagagttagaaggcacggcgacggcagcttcagcgacgagatgaacaccatcctggacaacctggccgccagagacttcatcaactggctgatccagaccaagatcaccgacggtggcggaggcggaggatctggtggtggtggatctggcggcggaggttctgacgcccacaaatctgaagtggcccaccggttcaaggacctgggcgaagagaatttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggctgcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaacctgggcaaagtgggcagcaagtgttgcaagcaccctgaggccaagagaatgccctgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaagtgctgcaaggccgacgacaaagagacctgcttcgccgaagagggcaagaaactggtggccgcttctcaggctgctctgggataataaggtacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 35 - DNA, 5219 bp, synthetic construct
  misc_feature 1..5219: note = synthetic construct
  source 1..5219: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccagaagccaccatggctcacgttcggggactgcagctgcctggatgtctggctcttgccgctctgtgtagcctggtgcacagccagcacgtgtttctggctcctcagcaagccagatcactgctgcagagagttagaaggcacggcgacggcagcttcagcgacgagatgaacaccatcctggacaacctggccgccagagacttcatcaactggctgatccagaccaagatcaccgacggtggcggaggcggaggatctggtggtggtggatctggcggcggaggaagttctcctgctggcagccctacaagcaccgaggaaggcacaagcgagtctgccacacctgagtctggccctggcacatctacagagcctagcgaaggatctgccccaggatctcctgccggctctccaacatctaccgaagagggaaccagcaccgagccatctgagggatctgctcccggaacaagcacagagccttcagaaggatccgctcctggcacctctgaaagcgccacaccagaaagcggacctggatctgagcctgccacaagcggatctgagacacctggaagcgagccagccacatctggcagcgaaacacctggttctccagccggatctcccaccagcacagaagagggcacatccgaatctgctacccctgaatctggaccaggcacctccacagaaccttccgagggttctgcccctggaacctctaccgaaccatcagaaggcagcgctccaggttcaccagctggaagcccaacctctacagaggaagggacatccactgagccaagcgagggaagcgctcccggcactagtacagaaccgagcgagggcagtgctcctggaaccagcgaatccgctactccagagagtggcccaggcaccagtactgaaccctctgagggtagcgcacccggaacatctgagagcgctactcccgaatcaggcccaggctctgaacctgctaccagcggaagtgaaacacccggcacctctactgagccctccgaaggctcagcacctggcacaagcactgaaccatcagagggctccgcaccaggcaccagcgaaagtgctacaccagagtcaggacccggaacctccgaaagtgcaactcctgagagcggaccaggctctcccgctggatctcctacatcaactgaagaagggacctccgagagcgcaaccccagagtctggtccaggatcagaacctgccacctccggctctgaaaccccaggcacttctgagtccgccacgccagaatctggtcctgggactagcaccgaaccgagtgaaggttcagctcccgggacttctacggaacccagtgaaggatctgcacccggcacatcaaccgaaccgtcagagggatcagcccctgggacttccacagagccgtctgagggcagcgccccagggacgtctacagaaccatctgaaggatcagcaccagggacctctaccgagccaagtgaaggcagtgcaccgggaagtccagcaggctcccctacaagtactgaagagggtactagcacggaacccagcgagggttccgctccagggacatctgaatccgcaactccggaatccggacctggcagtgaaccagctacatccggatccgagactccgggaacctcagaatcagctacacccgagagtggacctggctccgaaccagcaactagcggctcagaaactcctgggacaagcgagagtgcaacacccgaatctggacctggaacaagtactgagcccagcgaaggcagcgcccctggaacttctgaatctgccactcctgaaagtggccctggaagccctgcaggctcacccacatccacagaagaaggatcaccagcaggcagccccacttcaacggaagagggatccccagctggatccccaactagtacggaagaaggcacttcagaaagcgctacgcccgagtccggtcctggcacttctactgaaccatccgagggaagtgcccctggcacttccgagagtgctacacctgaaagcggtcccggctctgaaccagccacttctggatctgaaacgcccgggacatccgagtcagcaacgcccgaaagcggcccaggttccgagccggctactagtggttcagagactccagggacttccgagtctgctactcctgagtccggaccgggaacatcaaccgagccttccgaaggatctgcacctggaagccctgccggatctcctaccagtactgaggaaggcacctcagagtctgccactccagagtcaggtcctggaagcgaacctgcaacaagcggcagcgaaactccaggcactagcgagtcagctaccccagaatcaggacctggatctccagcagggtccccaacatctactgaggaaggctctcctgctggctcccctacctctaccgaagaggggacctcaacagagccatccgaggggagcgcacctggtacatcagagtccgcaactcccgagtctggccccggaactagcgaatctgcaaccccggaaagtggacccgggacgagtgaatcagccacacctgaatccggtccaggatccgagcctgcaacttctggaagcgagacaccaggatctgagccagctacgtctggctctgagactcctggatctcctgctggtagtcccacctccactgaagagggaacttccaccgaaccgagcgagggatcagcaccaggcactagcacagaaccgtccgaaggatctgctccaggctctgaacccgcaacctccggatcagaaacccctggaacatccgaaagcgctacaccggaaagtggccccggaacctctacagaacctagcgagggaagcgcaccaggataataaggtacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 36 - DNA, 3326 bp, synthetic construct
  misc_feature 1..3326: note = synthetic construct
  source 1..3326: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcggggactgcagctgcctggatgtctggctcttgccgctctgtgtagcctggtgcacagccagcatgtgtttctggctcctcagcaggccctgagcctgctgcaaagagttagaaggcacggcgacggcagcttcagcgacgagatgaataccgtgctggtggacaacctggccaccagagacttcatcaactggctgatccagaccaagatcaccgacggtggtggcggaggcggaggatctggtggcggtggttctggcggtggcggatctgctgagtttacccctccttgtcctccctgtcctgctccagaactgctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgcccagacaaagcccagagagcggcagttcaacagcacctacagagtggtgtccgtgctgaccgtgacacaccaggattggctgaacggcaaagagtacacctgtaaagtctccaacaagggcctgcctgctcctatcgagaaaaccatcagcaaggccaagggccagcctagagaaccccaggtgtacatcctgcctccacctcaagaggaactgaccaagaaccaggtgtccctgacctgtctggtcaccggcttctacccttccgatatcgccgtggagtgggagagcaacggacagcccgagaacacctacaagaccacacctccagtgctggacagcgacggctcttacctgctgtactccaagctgacagtgaacaagagccggtggcagcccggcaacatcttcacctgttctgtgatgcacgaggccctgcacaaccactacacccagaaaagcctgagcgtgtcccctggataataaggtacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 37 - DNA, 4382 bp, synthetic construct
  misc_feature 1..4382: note = synthetic construct
  source 1..4382: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccagaagccaccatggctcacgttcggggactgcagctgcctggatgtctggctcttgccgctctgtgtagcctggtgcacagccagcatgtgtttctggctcctcagcaagccctgagcctgctgcaaagagttagaaggcacggcgacggcagcttcagcgacgagatgaataccgtgctggtggacaacctggccaccagagacttcatcaactggctgatccagaccaagatcaccggtggtggcggaggcggaggatctggtggcggtggttctggcggtggcggatctgatacacacaagtctgaggtggcccaccggttcaaggacctgggcgaagaacacttcaaaggcctggtgctggtcgccttcagccagtacctgcagcagtgccctttcgaggaacacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctccactcgtcagacccgaagtggacgtgatgtgcaccgccttccacgacaatgaggccaccttcctgaagaaatacctgtacgaggtggccagacggcacccctacttttacgcccctgaactgctgttctttgccgccaggtacaaggccgccttcgccgaatgttgtcaggccgctgataaggccgcttgcctgctgcctaagctggacgagcttagagacgagggcaaagccagctccgccaagcagagactgaagtgtgccagcctgcagaagttcggcgatagagcctttaaggcctgggccgtcgctagactgagccagaagtttcccaaggccgagtttgccgaggtgtccaagctcgtgaccgacctgacaaaggtgcacaccgagtgctgtcacggcgacctgctggaatgcgccgacgatagagccgacctggccaagtacatgtgcgagaaccaggacagcatcagcagcaagctgaaagagtgctgcgacaagcctctgctggaaaagagccactgtctggccgaggtggaaaacgacgagatgcccgccgatctgccttctctggccgccgattacgtggaaagcaaggacgtgtgcaagaactacgccgaggccaaggacgtgttcctgggcatgtttctgtacgagtacgcccgcagacaccccgactactctgttatgctgctgctgagactggccaaggcctacgaggccactctggaaaagtgttgtgccgccgctgatccccacgagtgttacgccaaagtgttcgacgagttccagccactggtggaagaaccccagaacctggtcaagcagaactgcgagctgttcgagcagctgggcgagtacaagttccagaacgccctgctcgtgcggtacaccaagaaggtgccccaggtttccacacctacactggttgaggtgtcccggaacctgggaaaagtgggcgccaagtgttgcaagctgcctgaggccaagagaatgccctgcgccgaggattacctgagcgtggtgctgaacagactgtgcgtgctgcacgagaaaacccctgtgtccgagaaagtgaccaagtgctgtaccgagagcctggtcaatcggaggccttgctttagcgccctggaactggacgaggcctacgtgcccaaggccttcaacgccgagacattcaccttccacgccgacatgtgtaccctgagcgagaaagaaaagcaagtgaagaaacagacagccctggtcgagctggttaagcacaagcctaaggccaccaaagaacaactgaagggcgtgatggacaacttcgccgcctttgtggaaaaatgctgcaaggccgacgacaaagaggcctgcttcgcagaagagggccctaagtttgtggccgcctctcaagctgctctggcttaataaggtacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 38 - DNA, 5222 bp, synthetic construct
  misc_feature 1..5222: note = synthetic construct
  source 1..5222: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccagaagccaccatggctcacgttcggggactgcagctgcctggatgtctggctcttgccgctctgtgtagcctggtgcacagccagcatgtgtttctggctcctcagcaagccctgagcctgctgcaaagagttagaaggcacggcgacggcagcttcagcgacgagatgaataccgtgctggtggacaacctggccaccagagacttcatcaactggctgatccagaccaagatcaccgacggtggcggaggcggaggatctggtggtggtggatctggcggcggaggaagttctcctgctggcagccctacaagcaccgaggaaggcacaagcgagtctgccacacctgagtctggccctggcacatctacagagcctagcgaaggatctgccccaggatctcctgccggctctccaacatctaccgaagagggaaccagcaccgagccatctgagggatctgctcccggaacaagcacagagccttcagaaggatccgctcctggcacctctgaaagcgccacaccagaaagcggacctggctctgaacctgccacaagcggatctgagacacctggaagcgagccagccacatctggcagcgaaacacctggatcaccagccggatctcccacctctaccgaggaagggacatccgagagcgctaccccagaatctggaccaggcaccagcacagaaccctctgaaggttcagcccctggaacctctaccgaaccatcagaaggcagcgctccaggttctcccgctggatcccctacatccacagaagagggcacctccactgaacctagcgagggaagtgctcccggcacttccacagaaccatccgagggcagtgcacctggaaccagcgaatctgctacccctgagagtggacccggaacatccactgagccctccgagggttcagctccaggcacatcagaatccgccactccagagtccggaccaggatctgagccagctaccagcggctctgaaacacccggcactagtaccgagccaagcgagggtagcgcaccagggacaagtaccgaaccgtctgagggctccgcaccaggcacttccgaaagtgctactcctgaaagcggcccaggcactagcgaatccgcaacacccgagagcggtcctggaagtcctgcaggttcacctaccagcactgaagaggggactagcgagagcgcaactcctgaatcaggccctggatccgaacctgctacctccggaagtgaaacccctgggacaagcgaaagtgcaacgcccgagtcaggacccgggactagcacggaacccagtgaaggatctgcacccgggacatctaccgagccgtcagaaggttctgctccagggactagtactgagccttccgaaggttctgcacctggaacttccacagagcccagtgaaggcagtgcccctggcacaagcactgaaccgtccgaaggcagtgctcccgggaccagtacagaaccgagcgagggctctgctcctggtagtccagcaggatctccaactagcaccgaagaagggacttccaccgagccttccgagggaagcgctcctggaacatccgagtccgctacgccagagagtggcccaggttctgaacccgctacttccggctcagagactcctgggacttctgagtctgcaaccccggaaagtggtcctggtagcgaaccagcaactagcggaagcgagacacccggaacctcagagagtgctacaccggaatccggtccagggacgtctacggaaccgtctgaaggatcagctcccggcactagcgaaagcgctacacctgaaagtggtcccggatctccagcaggcagcccaacctctactgaagaaggttccccagctggaagccccacttccactgaggaaggctctcccgcaggctcacccactagtacggaagaaggcacgtccgagtctgctactcccgaatccggacctggaactagcactgagccaagcgaaggatcagcacccggaacctctgagtccgccacaccagaatctggtcctggttccgagcctgccacttcaggatcagaaaccccgggcacgagtgaatcagcaacgccggaatctggccccggaagcgaaccggctacgtctggatctgaaacgccagggacctccgaatcagctacgcctgagtctggtccagggacatccaccgaacctagtgaaggctccgcacctggaagccctgctggaagcccaacgagtactgaagagggcacttctgagagcgctacgcctgagtcaggacctggaagcgaacctgcaacatccggctcagaaacaccagggaccagcgaaagcgcaaccccagagagtggacctggatctccagctggctctcctactagtacagaggaaggcagccctgctggctccccaacgtcaacagaagaaggtactagcacagagcccagcgagggttccgctccgggaacttctgaatctgctacacccgagtcaggtcctggtacaagcgagtcagctacgcccgaaagtggacctggcacctcagagtctgcaactcctgagagcggtccaggatcagaaccagccacctctggctctgagacaccaggttctgagcctgcaacgtccggaagcgaaacaccaggcagtcctgccggaagtcctacttcaaccgaagaggggacctctacagagccatcagagggctctgcaccgggcacctcaacagaaccatctgaaggatccgcaccgggctctgagcctgctactagtggaagcgaaactcctggcaccagtgaatccgctactcccgagtctggcccgggaacgtctactgaaccatctgagggaagtgccccaggctaataaggtacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 39 - DNA, 3611 bp, synthetic construct
  misc_feature 1..3611: note = synthetic construct
  source 1..3611: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgccctgcagcgggaattgccaccatggctcacgttcggggactgcagctgcctggatgtctggctcttgccgctctgtgtagcctggtgcacagccagcatgtgtttctggctcctcagcaggccctgagcctgctgcaaagagttagaaggcacggcgacggcagcttcagcgacgagatgaataccgtgctggtggacaacctggccaccagagacttcatcaactggctgatccagaccaagatcaccgacggtggtggcggaggcggaggatctggtggcggtggttctggcggtggcggatctgctgagtttacccctccttgtcctccctgtcctgctccagaactgctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgcccagacaaagcccagagagcggcagttcaacagcacctacagagtggtgtccgtgctgaccgtgacacaccaggattggctgaacggcaaagagtacacctgtaaagtctccaacaagggcctgcctgctcctatcgagaaaaccatcagcaaggccaagggccagcctagagaaccccaggtgtacatcctgcctccacctcaagaggaactgaccaagaaccaggtgtccctgacctgtctggtcaccggcttctacccttccgatatcgccgtggagtgggagagcaacggacagcccgagaacacctacaagaccacacctccagtgctggacagcgacggctcttacctgctgtactccaagctgacagtgaacaagagccggtggcagcccggcaacatcttcacctgttctgtgatgcacgaggccctgcacaaccactacacccagaaaagcctgagcgtgtcccctggataataaggtaccgatctttttccctctgccaaaaattatggggacatcatgaagccccttgagcatctgacttctggctaataaaggaaatttattttcattgcaatagtgtgttggaattttttgtgtctctcactcggaaggcgcgaccggttgggcatggccaggtagcctatgctgtgtctggacgtcctcctgctggtatagttattttaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactcatcgattccaattcagcgggggccacctgatgtcccggccagcagaggaagcaacgcggggaccacgggttaacccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccaggggccagcccgaagatggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggttcctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacataccgggcaccctggcgcttcgccagccattcctgcaccagattcttcccgtccagcctggtcccaccttggctgtagtcatctgggtactcagggtctggggttcccatgcggaacatgtactttcggcctccacctaggatcacgtcaatgtccatgttggagatgagctgcgtagcgatgtcctggcacccctcctggcgggccgaggcaggcacgtcggcgtccgagtaccagttgcggttcaccgtgtgggcgtaggtgccggctggcgaggcgtgctgcactcgtgtggtggttaccactcccactgacttccctgctttcttggcccgattcatcacggagatgacctcgttgccgcgtgtcgtgttgcactggttaaagcgggcggctgcactcaagccaatggtctggaagttgcccttgaccccgcacaggtaggccgtggctgtggctccactgtctggcacatgtttgtctacattgtatgtcttggacagagccacatatgggaagcggtccatggccaggggtatctcaggccccagtttgtccttcttctgcccttttaggatctacgtcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 40 - DNA, 4667 bp, synthetic construct
  misc_feature 1..4667: note = synthetic construct
  source 1..4667: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattccagaagccaccatggctcacgttcggggactgcagctgcctggatgtctggctcttgccgctctgtgtagcctggtgcacagccagcatgtgtttctggctcctcagcaagccctgagcctgctgcaaagagttagaaggcacggcgacggcagcttcagcgacgagatgaataccgtgctggtggacaacctggccaccagagacttcatcaactggctgatccagaccaagatcaccggtggtggcggaggcggaggatctggtggcggtggttctggcggtggcggatctgatacacacaagtctgaggtggcccaccggttcaaggacctgggcgaagaacacttcaaaggcctggtgctggtcgccttcagccagtacctgcagcagtgccctttcgaggaacacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctccactcgtcagacccgaagtggacgtgatgtgcaccgccttccacgacaatgaggccaccttcctgaagaaatacctgtacgaggtggccagacggcacccctacttttacgcccctgaactgctgttctttgccgccaggtacaaggccgccttcgccgaatgttgtcaggccgctgataaggccgcttgcctgctgcctaagctggacgagcttagagacgagggcaaagccagctccgccaagcagagactgaagtgtgccagcctgcagaagttcggcgatagagcctttaaggcctgggccgtcgctagactgagccagaagtttcccaaggccgagtttgccgaggtgtccaagctcgtgaccgacctgacaaaggtgcacaccgagtgctgtcacggcgacctgctggaatgcgccgacgatagagccgacctggccaagtacatgtgcgagaaccaggacagcatcagcagcaagctgaaagagtgctgcgacaagcctctgctggaaaagagccactgtctggccgaggtggaaaacgacgagatgcccgccgatctgccttctctggccgccgattacgtggaaagcaaggacgtgtgcaagaactacgccgaggccaaggacgtgttcctgggcatgtttctgtacgagtacgcccgcagacaccccgactactctgttatgctgctgctgagactggccaaggcctacgaggccactctggaaaagtgttgtgccgccgctgatccccacgagtgttacgccaaagtgttcgacgagttccagccactggtggaagaaccccagaacctggtcaagcagaactgcgagctgttcgagcagctgggcgagtacaagttccagaacgccctgctcgtgcggtacaccaagaaggtgccccaggtttccacacctacactggttgaggtgtcccggaacctgggaaaagtgggcgccaagtgttgcaagctgcctgaggccaagagaatgccctgcgccgaggattacctgagcgtggtgctgaacagactgtgcgtgctgcacgagaaaacccctgtgtccgagaaagtgaccaagtgctgtaccgagagcctggtcaatcggaggccttgctttagcgccctggaactggacgaggcctacgtgcccaaggccttcaacgccgagacattcaccttccacgccgacatgtgtaccctgagcgagaaagaaaagcaagtgaagaaacagacagccctggtcgagctggttaagcacaagcctaaggccaccaaagaacaactgaagggcgtgatggacaacttcgccgcctttgtggaaaaatgctgcaaggccgacgacaaagaggcctgcttcgcagaagagggccctaagtttgtggccgcctctcaagctgctctggcttaataaggtaccgatctttttccctctgccaaaaattatggggacatcatgaagccccttgagcatctgacttctggctaataaaggaaatttattttcattgcaatagtgtgttggaattttttgtgtctctcactcggaaggcgcgaccggttgggcatggccaggtagcctatgctgtgtctggacgtcctcctgctggtatagttattttaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactcatcgattccaattcagcgggggccacctgatgtcccggccagcagaggaagcaacgcggggaccacgggttaacccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccaggggccagcccgaagatggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggttcctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacataccgggcaccctggcgcttcgccagccattcctgcaccagattcttcccgtccagcctggtcccaccttggctgtagtcatctgggtactcagggtctggggttcccatgcggaacatgtactttcggcctccacctaggatcacgtcaatgtccatgttggagatgagctgcgtagcgatgtcctggcacccctcctggcgggccgaggcaggcacgtcggcgtccgagtaccagttgcggttcaccgtgtgggcgtaggtgccggctggcgaggcgtgctgcactcgtgtggtggttaccactcccactgacttccctgctttcttggcccgattcatcacggagatgacctcgttgccgcgtgtcgtgttgcactggttaaagcgggcggctgcactcaagccaatggtctggaagttgcccttgaccccgcacaggtaggccgtggctgtggctccactgtctggcacatgtttgtctacattgtatgtcttggacagagccacatatgggaagcggtccatggccaggggtatctcaggccccagtttgtccttcttctgcccttttaggatctacgtcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 41 - DNA, 3611 bp, synthetic construct
  misc_feature 1..3611: note = synthetic construct
  source 1..3611: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgccctgcagcgggaattgccaccatggctcacgttcggggactgcagctgcctggatgtctggctcttgccgctctgtgtagcctggtgcacagccagcacgtgtttctggctcctcagcaagccagatcactgctgcagagagttagaaggcacggcgacggcagcttcagcgacgagatgaacaccatcctggacaacctggccgccagagacttcatcaactggctgatccagaccaagatcaccgacggtggcggaggcggaggatctggtggtggtggatctggcggcggaggttctgccgagtctaagtacggacctccttgtcctccctgtcctgctccagaagctgctggcggcccatccgtgtttctgttccctccaaagcctaaggacaccctgatgatcagcagaacccctgaagtgacctgcgtggtggtcgacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggactccgatggctcattcttcctgtacagcagactgaccgtggacaagagcaggtggcaagagggcaacgtgttcagctgcagcgtgatgcacgaggccctgcacaaccactacacccagaaaagcctgagcctgtctctgggctaataaggtaccgatctttttccctctgccaaaaattatggggacatcatgaagccccttgagcatctgacttctggctaataaaggaaatttattttcattgcaatagtgtgttggaattttttgtgtctctcactcggaaggcgcgaccggttgggcatggccaggtagcctatgctgtgtctggacgtcctcctgctggtatagttattttaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactcatcgattccaattcagcgggggccacctgatgtcccggccagcagaggaagcaacgcggggaccacgggttaacccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccaggggccagcccgaagatggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggttcctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacataccgggcaccctggcgcttcgccagccattcctgcaccagattcttcccgtccagcctggtcccaccttggctgtagtcatctgggtactcagggtctggggttcccatgcggaacatgtactttcggcctccacctaggatcacgtcaatgtccatgttggagatgagctgcgtagcgatgtcctggcacccctcctggcgggccgaggcaggcacgtcggcgtccgagtaccagttgcggttcaccgtgtgggcgtaggtgccggctggcgaggcgtgctgcactcgtgtggtggttaccactcccactgacttccctgctttcttggcccgattcatcacggagatgacctcgttgccgcgtgtcgtgttgcactggttaaagcgggcggctgcactcaagccaatggtctggaagttgcccttgaccccgcacaggtaggccgtggctgtggctccactgtctggcacatgtttgtctacattgtatgtcttggacagagccacatatgggaagcggtccatggccaggggtatctcaggccccagtttgtccttcttctgcccttttaggatctacgtcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 42 - DNA, 4664 bp, synthetic construct
  misc_feature 1..4664: note = synthetic construct
  source 1..4664: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattccagaagccaccatggctcacgttcggggactgcagctgcctggatgtctggctcttgccgctctgtgtagcctggtgcacagccagcacgtgtttctggctcctcagcaagccagatcactgctgcagagagttagaaggcacggcgacggcagcttcagcgacgagatgaacaccatcctggacaacctggccgccagagacttcatcaactggctgatccagaccaagatcaccgacggtggcggaggcggaggatctggtggtggtggatctggcggcggaggttctgacgcccacaaatctgaagtggcccaccggttcaaggacctgggcgaagagaatttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggctgcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaacctgggcaaagtgggcagcaagtgttgcaagcaccctgaggccaagagaatgccctgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaagtgctgcaaggccgacgacaaagagacctgcttcgccgaagagggcaagaaactggtggccgcttctcaggctgctctgggataataaggtaccgatctttttccctctgccaaaaattatggggacatcatgaagccccttgagcatctgacttctggctaataaaggaaatttattttcattgcaatagtgtgttggaattttttgtgtctctcactcggaaggcgcgaccggttgggcatggccaggtagcctatgctgtgtctggacgtcctcctgctggtatagttattttaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactcatcgattccaattcagcgggggccacctgatgtcccggccagcagaggaagcaacgcggggaccacgggttaacccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccaggggccagcccgaagatggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggttcctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacataccgggcaccctggcgcttcgccagccattcctgcaccagattcttcccgtccagcctggtcccaccttggctgtagtcatctgggtactcagggtctggggttcccatgcggaacatgtactttcggcctccacctaggatcacgtcaatgtccatgttggagatgagctgcgtagcgatgtcctggcacccctcctggcgggccgaggcaggcacgtcggcgtccgagtaccagttgcggttcaccgtgtgggcgtaggtgccggctggcgaggcgtgctgcactcgtgtggtggttaccactcccactgacttccctgctttcttggcccgattcatcacggagatgacctcgttgccgcgtgtcgtgttgcactggttaaagcgggcggctgcactcaagccaatggtctggaagttgcccttgaccccgcacaggtaggccgtggctgtggctccactgtctggcacatgtttgtctacattgtatgtcttggacagagccacatatgggaagcggtccatggccaggggtatctcaggccccagtttgtccttcttctgcccttttaggatctacgtcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 43 - DNA, 2911 bp, synthetic construct
  misc_feature 1..2911: note = synthetic construct
  source 1..2911: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcggggactgcagctgcctggatgtctggctcttgccgctctgtgtagcctggtgcacagccagcacgtgtttctggctcctcagcaagccagatcactgctgcagagagttagaaggcacggcgacggcagcttcagcgacgagatgaacaccatcctggacaacctggccgccagagacttcatcaactggctgatccagaccaagatcaccgacggtggcggaggcggaggatctggtggtggtggatctggcggcggaggttctgccgagtctaagtacggacctccttgtcctccctgtcctgctccagaagctgctggcggcccatccgtgtttctgttccctccaaagcctaaggacaccctgatgatcagcagaacccctgaagtgacctgcgtggtggtcgacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggactccgatggctcattcttcctgtacagcagactgaccgtggacaagagcaggtggcaagagggcaacgtgttcagctgcagcgtgatgcacgaggccctgcacaaccactacacccagaaaagcctgagcctgtctctgggctaataaggtacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 44 - DNA, 3964 bp, synthetic construct
  misc_feature 1..3964: note = synthetic construct
  source 1..3964: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccagaagccaccatggctcacgttcggggactgcagctgcctggatgtctggctcttgccgctctgtgtagcctggtgcacagccagcacgtgtttctggctcctcagcaagccagatcactgctgcagagagttagaaggcacggcgacggcagcttcagcgacgagatgaacaccatcctggacaacctggccgccagagacttcatcaactggctgatccagaccaagatcaccgacggtggcggaggcggaggatctggtggtggtggatctggcggcggaggttctgacgcccacaaatctgaagtggcccaccggttcaaggacctgggcgaagagaatttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggctgcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaacctgggcaaagtgggcagcaagtgttgcaagcaccctgaggccaagagaatgccctgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaagtgctgcaaggccgacgacaaagagacctgcttcgccgaagagggcaagaaactggtggccgcttctcaggctgctctgggataataaggtacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 45 - DNA, 2911 bp, synthetic construct; identical to SEQ ID NO: 43
  misc_feature 1..2911: note = synthetic construct
  source 1..2911: mol_type = other DNA

SEQ ID NO: 46 - DNA, 2911 bp, synthetic construct
  misc_feature 1..2911: note = synthetic construct
  source 1..2911: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcggggactgcagctgcctggatgtctggctcttgccgctctgtgtagcctggtgcacagccagcatgtgtttctggctcctcagcaggccctgagcctgctgcaaagagttagaaggcacggcgacggcagcttcagcgacgagatgaataccgtgctggtggacaacctggccaccagagacttcatcaactggctgatccagaccaagatcaccgacggtggtggcggaggcggaggatctggtggcggtggttctggcggtggcggatctgctgagtttacccctccttgtcctccctgtcctgctccagaactgctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgcccagacaaagcccagagagcggcagttcaacagcacctacagagtggtgtccgtgctgaccgtgacacaccaggattggctgaacggcaaagagtacacctgtaaagtctccaacaagggcctgcctgctcctatcgagaaaaccatcagcaaggccaagggccagcctagagaaccccaggtgtacatcctgcctccacctcaagaggaactgaccaagaaccaggtgtccctgacctgtctggtcaccggcttctacccttccgatatcgccgtggagtgggagagcaacggacagcccgagaacacctacaagaccacacctccagtgctggacagcgacggctcttacctgctgtactccaagctgacagtgaacaagagccggtggcagcccggcaacatcttcacctgttctgtgatgcacgaggccctgcacaaccactacacccagaaaagcctgagcgtgtcccctggataataaggtacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 47 - DNA, 3967 bp, synthetic construct
  misc_feature 1..3967: note = synthetic construct
  source 1..3967: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccagaagccaccatggctcacgttcggggactgcagctgcctggatgtctggctcttgccgctctgtgtagcctggtgcacagccagcatgtgtttctggctcctcagcaagccctgagcctgctgcaaagagttagaaggcacggcgacggcagcttcagcgacgagatgaataccgtgctggtggacaacctggccaccagagacttcatcaactggctgatccagaccaagatcaccggtggtggcggaggcggaggatctggtggcggtggttctggcggtggcggatctgatacacacaagtctgaggtggcccaccggttcaaggacctgggcgaagaacacttcaaaggcctggtgctggtcgccttcagccagtacctgcagcagtgccctttcgaggaacacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctccactcgtcagacccgaagtggacgtgatgtgcaccgccttccacgacaatgaggccaccttcctgaagaaatacctgtacgaggtggccagacggcacccctacttttacgcccctgaactgctgttctttgccgccaggtacaaggccgccttcgccgaatgttgtcaggccgctgataaggccgcttgcctgctgcctaagctggacgagcttagagacgagggcaaagccagctccgccaagcagagactgaagtgtgccagcctgcagaagttcggcgatagagcctttaaggcctgggccgtcgctagactgagccagaagtttcccaaggccgagtttgccgaggtgtccaagctcgtgaccgacctgacaaaggtgcacaccgagtgctgtcacggcgacctgctggaatgcgccgacgatagagccgacctggccaagtacatgtgcgagaaccaggacagcatcagcagcaagctgaaagagtgctgcgacaagcctctgctggaaaagagccactgtctggccgaggtggaaaacgacgagatgcccgccgatctgccttctctggccgccgattacgtggaaagcaaggacgtgtgcaagaactacgccgaggccaaggacgtgttcctgggcatgtttctgtacgagtacgcccgcagacaccccgactactctgttatgctgctgctgagactggccaaggcctacgaggccactctggaaaagtgttgtgccgccgctgatccccacgagtgttacgccaaagtgttcgacgagttccagccactggtggaagaaccccagaacctggtcaagcagaactgcgagctgttcgagcagctgggcgagtacaagttccagaacgccctgctcgtgcggtacaccaagaaggtgccccaggtttccacacctacactggttgaggtgtcccggaacctgggaaaagtgggcgccaagtgttgcaagctgcctgaggccaagagaatgccctgcgccgaggattacctgagcgtggtgctgaacagactgtgcgtgctgcacgagaaaacccctgtgtccgagaaagtgaccaagtgctgtaccgagagcctggtcaatcggaggccttgctttagcgccctggaactggacgaggcctacgtgcccaaggccttcaacgccgagacattcaccttccacgccgacatgtgtaccctgagcgagaaagaaaagcaagtgaagaaacagacagccctggtcgagctggttaagcacaagcctaaggccaccaaagaacaactgaagggcgtgatggacaacttcgccgcctttgtggaaaaatgctgcaaggccgacgacaaagaggcctgcttcgcagaagagggccctaagtttgtggccgcctctcaagctgctctggcttaataaggtacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 48 - DNA, 4807 bp, synthetic construct
  misc_feature 1..4807: note = synthetic construct
  source 1..4807: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccagaagccaccatggctcacgttcggggactgcagctgcctggatgtctggctcttgccgctctgtgtagcctggtgcacagccagcatgtgtttctggctcctcagcaagccctgagcctgctgcaaagagttagaaggcacggcgacggcagcttcagcgacgagatgaataccgtgctggtggacaacctggccaccagagacttcatcaactggctgatccagaccaagatcaccgacggtggcggaggcggaggatctggtggtggtggatctggcggcggaggaagttctcctgctggcagccctacaagcaccgaggaaggcacaagcgagtctgccacacctgagtctggccctggcacatctacagagcctagcgaaggatctgccccaggatctcctgccggctctccaacatctaccgaagagggaaccagcaccgagccatctgagggatctgctcccggaacaagcacagagccttcagaaggatccgctcctggcacctctgaaagcgccacaccagaaagcggacctggctctgaacctgccacaagcggatctgagacacctggaagcgagccagccacatctggcagcgaaacacctggatcaccagccggatctcccacctctaccgaggaagggacatccgagagcgctaccccagaatctggaccaggcaccagcacagaaccctctgaaggttcagcccctggaacctctaccgaaccatcagaaggcagcgctccaggttctcccgctggatcccctacatccacagaagagggcacctccactgaacctagcgagggaagtgctcccggcacttccacagaaccatccgagggcagtgcacctggaaccagcgaatctgctacccctgagagtggacccggaacatccactgagccctccgagggttcagctccaggcacatcagaatccgccactccagagtccggaccaggatctgagccagctaccagcggctctgaaacacccggcactagtaccgagccaagcgagggtagcgcaccagggacaagtaccgaaccgtctgagggctccgcaccaggcacttccgaaagtgctactcctgaaagcggcccaggcactagcgaatccgcaacacccgagagcggtcctggaagtcctgcaggttcacctaccagcactgaagaggggactagcgagagcgcaactcctgaatcaggccctggatccgaacctgctacctccggaagtgaaacccctgggacaagcgaaagtgcaacgcccgagtcaggacccgggactagcacggaacccagtgaaggatctgcacccgggacatctaccgagccgtcagaaggttctgctccagggactagtactgagccttccgaaggttctgcacctggaacttccacagagcccagtgaaggcagtgcccctggcacaagcactgaaccgtccgaaggcagtgctcccgggaccagtacagaaccgagcgagggctctgctcctggtagtccagcaggatctccaactagcaccgaagaagggacttccaccgagccttccgagggaagcgctcctggaacatccgagtccgctacgccagagagtggcccaggttctgaacccgctacttccggctcagagactcctgggacttctgagtctgcaaccccggaaagtggtcctggtagcgaaccagcaactagcggaagcgagacacccggaacctcagagagtgctacaccggaatccggtccagggacgtctacggaaccgtctgaaggatcagctcccggcactagcgaaagcgctacacctgaaagtggtcccggatctccagcaggcagcccaacctctactgaagaaggttccccagctggaagccccacttccactgaggaaggctctcccgcaggctcacccactagtacggaagaaggcacgtccgagtctgctactcccgaatccggacctggaactagcactgagccaagcgaaggatcagcacccggaacctctgagtccgccacaccagaatctggtcctggttccgagcctgccacttcaggatcagaaaccccgggcacgagtgaatcagcaacgccggaatctggccccggaagcgaaccggctacgtctggatctgaaacgccagggacctccgaatcagctacgcctgagtctggtccagggacatccaccgaacctagtgaaggctccgcacctggaagccctgctggaagcccaacgagtactgaagagggcacttctgagagcgctacgcctgagtcaggacctggaagcgaacctgcaacatccggctcagaaacaccagggaccagcgaaagcgcaaccccagagagtggacctggatctccagctggctctcctactagtacagaggaaggcagccctgctggctccccaacgtcaacagaagaaggtactagcacagagcccagcgagggttccgctccgggaacttctgaatctgctacacccgagtcaggtcctggtacaagcgagtcagctacgcccgaaagtggacctggcacctcagagtctgcaactcctgagagcggtccaggatcagaaccagccacctctggctctgagacaccaggttctgagcctgcaacgtccggaagcgaaacaccaggcagtcctgccggaagtcctacttcaaccgaagaggggacctctacagagccatcagagggctctgcaccgggcacctcaacagaaccatctgaaggatccgcaccgggctctgagcctgctactagtggaagcgaaactcctggcaccagtgaatccgctactcccgagtctggcccgggaacgtctactgaaccatctgagggaagtgccccaggctaataaggtacctctagagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 49 - DNA, 1513 bp, synthetic construct
  misc_feature 1..1513: note = synthetic construct
  source 1..1513: mol_type = other DNA
acgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgccctgcagcgggaattgccaccatggctcacgttcggggactgcagctgcctggatgtctggctcttgccgctctgtgtagcctggtgcacagccagcatgtgtttctggctcctcagcaggccctgagcctgctgcaaagagttagaaggcacggcgacggcagcttcagcgacgagatgaataccgtgctggtggacaacctggccaccagagacttcatcaactggctgatccagaccaagatcaccgacggtggtggcggaggcggaggatctggtggcggtggttctggcggtggcggatctgctgagtttacccctccttgtcctccctgtcctgctccagaactgctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgcccagacaaagcccagagagcggcagttcaacagcacctacagagtggtgtccgtgctgaccgtgacacaccaggattggctgaacggcaaagagtacacctgtaaagtctccaacaagggcctgcctgctcctatcgagaaaaccatcagcaaggccaagggccagcctagagaaccccaggtgtacatcctgcctccacctcaagaggaactgaccaagaaccaggtgtccctgacctgtctggtcaccggcttctacccttccgatatcgccgtggagtgggagagcaacggacagcccgagaacacctacaagaccacacctccagtgctggacagcgacggctcttacctgctgtactccaagctgacagtgaacaagagccggtggcagcccggcaacatcttcacctgttctgtgatgcacgaggccctgcacaaccactacacccagaaaagcctgagcgtgtcccctggataataaggtaccgatctttttccctctgccaaaaattatggggacatcatgaagccccttgagcatctgacttctggctaataaaggaaatttattttcattgcaatagtgtgttggaattttttgtgtctctcactcg

SEQ ID NO: 50 - DNA, 2569 bp, synthetic construct
  misc_feature 1..2569: note = synthetic construct
  source 1..2569: mol_type = other DNA
acgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattccagaagccaccatggctcacgttcggggactgcagctgcctggatgtctggctcttgccgctctgtgtagcctggtgcacagccagcatgtgtttctggctcctcagcaagccctgagcctgctgcaaagagttagaaggcacggcgacggcagcttcagcgacgagatgaataccgtgctggtggacaacctggccaccagagacttcatcaactggctgatccagaccaagatcaccggtggtggcggaggcggaggatctggtggcggtggttctggcggtggcggatctgatacacacaagtctgaggtggcccaccggttcaaggacctgggcgaagaacacttcaaaggcctggtgctggtcgccttcagccagtacctgcagcagtgccctttcgaggaacacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctccactcgtcagacccgaagtggacgtgatgtgcaccgccttccacgacaatgaggccaccttcctgaagaaatacctgtacgaggtggccagacggcacccctacttttacgcccctgaactgctgttctttgccgccaggtacaaggccgccttcgccgaatgttgtcaggccgctgataaggccgcttgcctgctgcctaagctggacgagcttagagacgagggcaaagccagctccgccaagcagagactgaagtgtgccagcctgcagaagttcggcgatagagcctttaaggcctgggccgtcgctagactgagccagaagtttcccaaggccgagtttgccgaggtgtccaagctcgtgaccgacctgacaaaggtgcacaccgagtgctgtcacggcgacctgctggaatgcgccgacgatagagccgacctggccaagtacatgtgcgagaaccaggacagcatcagcagcaagctgaaagagtgctgcgacaagcctctgctggaaaagagccactgtctggccgaggtggaaaacgacgagatgcccgccgatctgccttctctggccgccgattacgtggaaagcaaggacgtgtgcaagaactacgccgaggccaaggacgtgttcctgggcatgtttctgtacgagtacgcccgcagacaccccgactactctgttatgctgctgctgagactggccaaggcctacgaggccactctggaaaagtgttgtgccgccgctgatccccacgagtgttacgccaaagtgttcgacgagttccagccactggtggaagaaccccagaacctggtcaagcagaactgcgagctgttcgagcagctgggcgagtacaagttccagaacgccctgctcgtgcggtacaccaagaaggtgccccaggtttccacacctacactggttgaggtgtcccggaacctgggaaaagtgggcgccaagtgttgcaagctgcctgaggccaagagaatgccctgcgccgaggattacctgagcgtggtgctgaacagactgtgcgtgctgcacgagaaaacccctgtgtccgagaaagtgaccaagtgctgtaccgagagcctggtcaatcggaggccttgctttagcgccctggaactggacgaggcctacgtgcccaaggccttcaacgccgagacattcaccttccacgccgacatgtgtaccctgagcgagaaagaaaagcaagtgaagaaacagacagccctggtcgagctggttaagcacaagcctaaggccaccaaagaacaactgaagggcgtgatggacaacttcgccgcctttgtggaaaaatgctgcaaggccgacgacaaagaggcctgcttcgcagaagagggccctaagtttgtggccgcctctcaagctgctctggcttaataaggtaccgatctttttccctctgccaaaaattatggggacatcatgaagccccttgagcatctgacttctggctaataaaggaaatttattttcattgcaatagtgtgttggaattttttgtgtctctcactcg

SEQ ID NO: 51 - DNA, 1513 bp, synthetic construct
  misc_feature 1..1513: note = synthetic construct
  source 1..1513: mol_type = other DNA
acgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgccctgcagcgggaattgccaccatggctcacgttcggggactgcagctgcctggatgtctggctcttgccgctctgtgtagcctggtgcacagccagcacgtgtttctggctcctcagcaagccagatcactgctgcagagagttagaaggcacggcgacggcagcttcagcgacgagatgaacaccatcctggacaacctggccgccagagacttcatcaactggctgatccagaccaagatcaccgacggtggcggaggcggaggatctggtggtggtggatctggcggcggaggttctgccgagtctaagtacggacctccttgtcctccctgtcctgctccagaagctgctggcggcccatccgtgtttctgttccctccaaagcctaaggacaccctgatgatcagcagaacccctgaagtgacctgcgtggtggtcgacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggactccgatggctcattcttcctgtacagcagactgaccgtggacaagagcaggtggcaagagggcaacgtgttcagctgcagcgtgatgcacgaggccctgcacaaccactacacccagaaaagcctgagcctgtctctgggctaataaggtaccgatctttttccctctgccaaaaattatggggacatcatgaagccccttgagcatctgacttctggctaataaaggaaatttattttcattgcaatagtgtgttggaattttttgtgtctctcactcg

SEQ ID NO: 52 - DNA, 2566 bp, synthetic construct
  misc_feature 1..2566: note = synthetic construct
  source 1..2566: mol_type = other DNA
acgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattccagaagccaccatggctcacgttcggggactgcagctgcctggatgtctggctcttgccgctctgtgtagcctggtgcacagccagcacgtgtttctggctcctcagcaagccagatcactgctgcagagagttagaaggcacggcgacggcagcttcagcgacgagatgaacaccatcctggacaacctggccgccagagacttcatcaactggctgatccagaccaagatcaccgacggtggcggaggcggaggatctggtggtggtggatctggcggcggaggttctgacgcccacaaatctgaagtggcccaccggttcaaggacctgggcgaagagaatttcaaggccctggtgctgatcgccttcgctcagtacctgcagcagtgccccttcgaggaccacgtgaagctggtcaacgaagtgaccgagttcgccaagacctgcgtggccgacgagagcgccgagaactgtgataagagcctgcacaccctgttcggcgacaagctgtgtacagtggccacactgagagaaacctacggcgagatggccgactgctgcgccaagcaagagcccgagagaaacgagtgcttcctgcagcacaaggacgacaaccccaacctgcctagactcgtgcggcctgaagtggacgtgatgtgcaccgccttccacgacaacgaggaaaccttcctgaagaagtacctgtacgagatcgccagacggcacccctacttttacgcccctgagctgctgttcttcgccaagcggtataaggccgccttcaccgagtgttgtcaggccgctgataaggctgcctgcctgctgcctaagctggacgagcttagagacgagggcaaagccagctccgccaagcagagactgaagtgtgccagcctgcagaagttcggcgagagagcctttaaggcctgggccgttgctagactgagccagagatttcccaaggccgagtttgccgaggtgtccaagctcgtgaccgacctgacaaaggtgcacaccgagtgctgccacggcgacctgctggaatgcgccgacgatagagccgacctggccaagtacatctgcgagaaccaggacagcatcagcagcaagctgaaagagtgctgcgagaagcctctgctggaaaagagccactgtatcgccgaggtggaaaacgacgagatgcccgccgatctgccttctctggccgccgattttgtggaaagcaaggacgtgtgcaagaactacgccgaggccaaggacgtgttcctgggcatgtttctgtacgagtacgcccgcagacaccccgactactctgttgtgctgctgctgagactggccaaaacctacgagacaaccctggaaaagtgctgtgccgccgctgatcctcacgagtgttacgccaaggtgttcgacgagttcaagccactggtggaagaaccccagaacctgatcaagcagaactgcgagctgttcgagcagctgggcgagtacaagttccagaacgccctgctcgtgcggtacaccaagaaggtgccccaggtttccacacctacactggttgaggtgtcccggaacctgggcaaagtgggcagcaagtgttgcaagcaccctgaggccaagagaatgccctgcgccgaggattacctgagcgtggtgctgaatcagctgtgcgtgctgcacgagaaaacccctgtgtccgacagagtgaccaagtgctgtaccgagagcctggtcaacagacggccttgctttagcgccctcgaggtggacgagacatacgtgcccaaagagttcaacgccgagacattcaccttccacgccgacatctgtaccctgagcgagaaagagcggcagatcaagaaacagactgccctggtggaactggtcaagcacaagcccaaggccaccaaagaacagctgaaggccgtgatggacgacttcgccgccttcgtggaaaagtgctgcaaggccgacgacaaagagacctgcttcgccgaagagggcaagaaactggtggccgcttctcaggctgctctgggataataaggtaccgatctttttccctctgccaaaaattatggggacatcatgaagccccttgagcatctgacttctggctaataaaggaaatttattttcattgcaatagtgtgttggaattttttgtgtctctcactcg

SEQ ID NO: 53 - DNA, 2211 bp, Adeno-Associated Virus hu68
  source 1..2211: mol_type = unassigned DNA
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 54 - DNA, 2211 bp, Adeno-Associated Virus hu68
  source 1..2211: mol_type = unassigned DNA
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

SEQ ID NO: 55 - protein, 736 aa, Adeno-Associated Virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL